(19) United States
(12) Patent Application Publication
(10) Pub. No.: US 2022/0175764 A1
(43) Pub. Date: Jun. 9, 2022

(54) USE OF METHYLNALTREXONE AND RIFAXIMIN FOR TREATMENT OF INCREASED GUT PERMEABILITY OR ASSOCIATED DISORDERS

(71) Applicant: Bausch Health Ireland Limited, Dublin (IE)

(72) Inventors: Lorin K. Johnson, Los Altos Hills, CA (US); XiaoJun Nicole Deng, San Francisco, CA (US); William J. Jo Siu, Emeryville, CA (US); Robert J. Israel, Suffern, NY (US); Zeev Heimanson, Calabasas, CA (US)

(73) Assignee: Bausch Health Ireland Limited, Dublin (IE)

(21) Appl. No.: 17/529,818

(22) Filed: Nov. 18, 2021

Related U.S. Application Data
(63) Continuation of application No. PCT/EP2020/065374, filed on Jun. 3, 2020.
(60) Provisional application No. 62/856,497, filed on Jun. 3, 2019.
(60) Provisional application No. 62/937,606, filed on Nov. 19, 2019.

(51) Int. Cl. A61K 31/485; A61K 31/437; A61P 1/16; A61P 1/00

(52) U.S. Cl. CPC A61K 31/485; A61P 1/00; A61P 1/16; A61K 31/437

(57) ABSTRACT

The present disclosure is directed to the use of methylnaltrexone, or a salt thereof, and rifaximin for the treatment of increased gut permeability, or an associated disorder, e.g., NASH or NAFLD.

### CROSS-REFERENCE TO RELATED APPLICATIONS

This application is a continuation of International Application No. PCT/EP2020/065374, filed on Jun. 3, 2020, which claims the benefit of U.S. Provisional Application No. 62/856,497, filed on Jun. 3, 2019, and 62/937,606, filed on Nov. 19, 2019, the entire contents of each of which are expressly incorporated herein by reference.

### BACKGROUND

Increased gut permeability (also known as GP or leaky gut) or an associated disorder occurs in the progression of numerous diseases such as irritable bowel syndrome (IBS), nonalcoholic fatty liver disease (NAFLD), nonalcoholic steatohepatitis (NASH), and hepatic encephalopathy (HE). These conditions are partially driven by liver proinflammatory processes activated by the translocation of bacteria and proinflammatory bacterial products leaking from the gut into the liver and subsequently into the bloodstream.

Leaky gut manifests when a break occurs in the gastrointestinal (GI) barrier, thereby exposing the inside of the body to the external environment present in the lumen of the GI tract. Leaky gut syndrome is also known as increased intestinal permeability, and as a result of breaks in the GI tract, objects that are not supposed to be absorbed through the GI tract and into the body are, in fact, allowed entry into the body. These foreign objects can be a single molecule, such as an incompletely digested food molecule, or can be as large as a pathogen, such as a bacteria or virus, or their products.

### SUMMARY OF INVENTION

The invention described herein provides new and efficacious therapies in the field for treating leaky gut syndrome and diseases related to, resulting from, or exacerbated by increased gut permeability in a subject.

In one aspect, the invention includes a method of treating leaky gut syndrome and/or symptoms thereof in a subject in need thereof. In some embodiments, the methods include administering: (i) a therapeutically effective amount of methylnaltrexone or a salt thereof and (ii) a therapeutically effective amount of rifaximin.

In another aspect, the invention includes a method of treating a disease or syndrome related to, resulting from, or exacerbated by leaky gut in a subject in need thereof, the method comprising administering: (i) a therapeutically effective amount of methylnaltrexone or a salt thereof and (ii) a therapeutically effective amount of rifaximin. In some embodiments, the disease or syndrome related to, resulting from, or exacerbated by leaky gut is selected from the group consisting of obesity, a fatty liver disease, celiac disease, Crohn's disease, and irritable bowel syndrome. In some embodiments, a fatty liver disease is NASH or NAFLD.

In some embodiments, the disease or syndrome related to, resulting from, or exacerbated by leaky gut is selected from the group consisting of irritable bowel syndrome, inflammatory bowel disease, Crohn's disease, ulcerative colitis, indeterminate colitis, chemotherapy-induced colitis, celiac disease, infectious diarrhea, atopy, allergy, food allergy, asthma, autism, chronic fatigue syndrome, lupus, metabolic syndromes, diabetes, type 1 diabetes, type 2 diabetes, hypertension, hyperlipidemia, neoplasia, cancer, idiopathic inflammatory conditions, rheumatoid arthritis, neurologic disorders, multiple sclerosis, migraines, psoriatic arthritic, autoimmune diseases, rheumatoid arthritis, skin disorders, psoriasis, eczema, acne, metabolic bone disease, osteoporosis in adults, primary growth failure in children, liver disease, fatty liver diseases, nonalcoholic steatohepatitis (NASH), nonalcoholic fatty liver disease (NAFLD), hepatic encephalopathy (RE), anklyosing spondylitis, obesity, graft versus host disease (GVHD), multiple organ dysfunction syndrome, Parkinson's disease, acute pancreatitis, fibromyalgia, mental illness, depression, schizophrenia, burn injury, heart failure, and renal failure.

In some embodiments, the disease or syndrome related to, resulting from, or exacerbated by leaky gut is a liver disease, e.g., a fatty liver disease, such as nonalcoholic steatohepatitis (NASH) or nonalcoholic fatty liver disease (NAFLD).

In some embodiments, the disease or syndrome related to, resulting from, or exacerbated by leaky gut is cirrhosis.

In some embodiments, the disease or syndrome related to, resulting from, or exacerbated by leaky gut is hepatic encephalopathy (HE).

In another aspect, the invention includes a method of treating NASH in a subject in need thereof, the method comprising administering: (i) methylnaltrexone or a salt thereof; and (ii) rifaximin.

In another aspect, the invention includes a method of treating NAFLD in a subject in need thereof, the method comprising administering: (i) methylnaltrexone or a salt thereof and (ii) rifaximin.

In another aspect, the invention includes a method of treating HE in a subject in need thereof, the method comprising administering: (i) methylnaltrexone or a salt thereof; and, optionally, (ii) rifaximin. In some embodiments, treating HE comprises reducing the risk of overt HE recurrence in the subject.

In another aspect, the invention includes a method of treating increased gut permeability in a subject in need thereof, the method comprising administering a therapeutic agent selected from the group consisting of methylnaltrexone, or a salt thereof, rifaximin, and a combination thereof.

In another aspect, the invention includes a method of reducing gut permeability in a subject in need thereof, the method comprising administering a therapeutic agent selected from the group consisting of methylnaltrexone or a salt thereof, rifaximin, and a combination thereof.

In another aspect, the invention includes a method of treating a disease or condition associated with increased gut permeability in a subject in need thereof, the method comprising administering a therapeutic agent selected from the group consisting of methylnaltrexone, or a salt thereof, rifaximin, and a combination thereof.

In some embodiments, the disease or condition associated with increased gut permeability is selected from the group consisting of irritable bowel syndrome, inflammatory bowel disease, Crohn's disease, ulcerative colitis, indeterminate colitis, chemotherapy-induced colitis, celiac disease, infectious diarrhea, atopy, allergy, food allergy, asthma, autism, chronic fatigue syndrome, lupus, metabolic syndromes, diabetes, type 1 diabetes, type 2 diabetes, hypertension, hyperlipidemia, neoplasia, cancer, idiopathic inflammatory conditions, rheumatoid arthritis, neurologic disorders, multiple sclerosis, migraines, psoriatic arthritic, autoimmune diseases, rheumatoid arthritis, skin disorders, psoriasis, eczema, acne, metabolic bone disease, osteoporosis in adults, primary growth failure in children, liver disease, fatty liver diseases, nonalcoholic steatohepatitis (NASH), nonalcoholic fatty liver disease (NAFLD), hepatic encephalopathy (HE), anklyosing spondylitis, obesity, graft versus host disease (GVHD), multiple organ dysfunction syndrome, Parkinson's disease, acute pancreatitis, fibromyalgia, mental illness, depression, schizophrenia, burn injury, heart failure, and renal failure.

In some embodiments, the disease or condition associated with increased gut permeability is a liver disease, e.g., a fatty liver disease, such as NASH or NAFLD.

In some embodiments, the fatty liver disease is NASH.

In some embodiments, the fatty liver disease is NAFLD.

In some embodiments, the disease or condition associated with increased gut permeability is hepatic encephalopathy (HE).

In yet another aspect, the invention includes a method of treating NASH or NAFLD or a symptom thereof in a subject in need thereof, the method comprising administering a therapeutic agent selected from the group consisting of methylnaltrexone, or a salt thereof, rifaximin, and a combination thereof.

In yet another aspect, the invention includes a method of treating HE or a symptom thereof in a subject in need thereof, the method comprising administering (i) methylnaltrexone, or a salt thereof or (ii) methylnaltrexone, or a salt thereof, and rifaximin.

In another aspect, the invention includes a method of treating NASH in a subject in need thereof, the method comprising administering: (i) a therapeutically effective amount of methylnaltrexone or a salt thereof; and (ii) a therapeutically effective amount of rifaximin.

In another aspect, the invention includes a method of treating NAFLD in a subject in need thereof, the method comprising administering: (i) a therapeutically effective amount of methylnaltrexone or a salt thereof; and (ii) a therapeutically effective amount of rifaximin.

In another aspect, the invention includes a method of treating HE in a subject in need thereof, the method comprising administering: (i) a therapeutically effective amount of methylnaltrexone or a salt thereof and, optionally, (ii) a therapeutically effective amount of rifaximin. In some embodiments, treating HE comprises reducing the risk of overt HE recurrence in the subject.

In another aspect, the invention includes a method of treating increased gut permeability in a subject in need thereof, the method comprising administering a therapeutically effective amount of a therapeutic agent selected from the group consisting of methylnaltrexone, or a salt thereof, rifaximin, and a combination thereof.

In another aspect, the invention includes a method of reducing gut permeability in a subject in need thereof, the method comprising administering a therapeutically effective amount of a therapeutic agent selected from the group consisting of methylnaltrexone or a salt thereof, rifaximin, and a combination thereof.

In yet another aspect, the invention includes a method of treating NASH or NAFLD or a symptom thereof in a subject in need thereof, the method comprising administering a therapeutically effective amount of a therapeutic agent selected from the group consisting of methylnaltrexone, or a salt thereof, rifaximin, and a combination thereof.

In yet another aspect, the invention includes a method of treating HE or a symptom thereof in a subject in need thereof, the method comprising administering (i) a therapeutically effective amount of methylnaltrexone, or a salt thereof; or (ii) a therapeutically effective amount of methylnaltrexone, or a salt thereof, and rifaximin.

In some embodiments of the methods of the invention, administering a therapeutically effective amount of methylnaltrexone or a salt thereof comprises administering (i) about 0.5 mg to about 500 mg, about 0.5 mg to about 200 mg, about 50 mg to about 500 mg, or about 150 mg to about 450 mg; or (ii) about 1 mg/kg to about 100 mg/kg, about 5 mg/kg to about 75 mg/kg, or about 25 mg/kg to about 75 mg/kg of methylnaltrexone, or a salt thereof. In some embodiments, administering a therapeutically effective amount of methylnaltrexone or a salt thereof comprises administering oral RELISTOR or subcutaneous RELISTOR.

In some embodiments of the methods of the invention, administering a therapeutically effective amount of rifaximin comprises administering: (i) about 25 mg to about 1000 mg, about 300 mg to about 750 mg, or about 500 mg to about 600 mg, or (ii) about 100 mg/kg to about 700 mg/kg, about 250 mg/kg to about 550 mg/kg or about 350 mg/kg to about 450 mg/kg of rifaximin. In some embodiments, administering a therapeutically effective amount of rifaximin comprises administering XIFAXAN (550 mg) or XIFAXAN (200 mg).

In another aspect, the invention includes a pharmaceutical composition for treating increased gut permeability in a subject in need thereof, the pharmaceutical composition comprising a therapeutic agent selected from the group consisting of methylnaltrexone, or a salt thereof, rifaximin, and a combination thereof.

In another aspect, the invention includes a pharmaceutical composition for reducing gut permeability in a subject in need thereof, the pharmaceutical composition comprising a therapeutic agent selected from the group consisting of methylnaltrexone, or a salt thereof, rifaximin, and a combination thereof.

In another aspect, the invention includes a pharmaceutical composition for treating a disease or condition associated with increased gut permeability in a subject in need thereof, the pharmaceutical composition comprising a therapeutic agent selected from the group consisting of methylnaltrexone, or a salt thereof, rifaximin, and a combination thereof.

In another aspect, the invention includes a pharmaceutical composition for treating NASH or a symptom thereof in a subject in need thereof, the pharmaceutical composition comprising a therapeutic agent selected from the group consisting of methylnaltrexone, or a salt thereof, rifaximin, and a combination thereof.

In another aspect, the invention includes a pharmaceutical composition for treating NAFLD or a symptom thereof in a subject in need thereof, the pharmaceutical composition comprising a therapeutic agent selected from the group consisting of methylnaltrexone, or a salt thereof, rifaximin, and a combination thereof.

In a further aspect, the invention includes a pharmaceutical composition for treating leaky gut syndrome and/or symptoms thereof in a subject in need thereof. In some embodiments, the pharmaceutical compositions comprise: (i) methylnaltrexone or a salt thereof; (ii) rifaximin; and (iii) a pharmaceutically acceptable carrier. In some embodiments, the pharmaceutical compositions comprise: (i) a therapeutically effective amount of methylnaltrexone or a salt thereof; (ii) a therapeutically effective amount of rifaximin; and (iii) a pharmaceutically acceptable carrier.

In an additional aspect, the invention includes a pharmaceutical composition for treating a disease or syndrome related to, resulting from, or exacerbated by leaky gut in a subject in need thereof, comprising: (i) methylnaltrexone or a salt thereof; (ii) rifaximin; and (iii) a pharmaceutically acceptable carrier. In an additional aspect, the invention includes a pharmaceutical composition for treating a disease or syndrome related to, resulting from, or exacerbated by leaky gut in a subject in need thereof, comprising: (i) a therapeutically effective amount of methylnaltrexone or a salt thereof; (ii) a therapeutically effective amount of rifaximin; and (iii) a pharmaceutically acceptable carrier. In some embodiments, the disease or syndrome related to, resulting from, or exacerbated by leaky gut is selected from the group consisting of irritable bowel syndrome, inflammatory bowel disease, Crohn's disease, ulcerative colitis, indeterminate colitis, chemotherapy-induced colitis, celiac disease, infectious diarrhea, atopy, allergy, food allergy, asthma, autism, chronic fatigue syndrome, lupus, metabolic syndromes, diabetes, type 1 diabetes, type 2 diabetes, hypertension, hyperlipidemia, neoplasia, cancer, idiopathic inflammatory conditions, rheumatoid arthritis, neurologic disorders, multiple sclerosis, migraines, psoriatic arthritic, autoimmune diseases, rheumatoid arthritis, skin disorders, psoriasis, eczema, acne, metabolic bone disease, osteoporosis in adults, primary growth failure in children, liver disease, fatty liver diseases, nonalcoholic steatohepatitis (NASH), nonalcoholic fatty liver disease (NAFLD), hepatic encephalopathy (HE), anklyosing spondylitis, obesity, graft versus host disease (GVHD), multiple organ dysfunction syndrome, Parkinson's disease, acute pancreatitis, fibromyalgia, mental illness, depression, schizophrenia, burn injury, heart failure, and renal failure. In some embodiments, the disease or syndrome related to, resulting from, or exacerbated by leaky gut is selected from the group consisting of obesity, a fatty liver disease, celiac disease, Crohn's disease, and irritable bowel syndrome. In some embodiments, a fatty liver disease is NASH or NAFLD. In some embodiments, the pharmaceutical composition comprises: (i) about 0.5 mg to about 500 mg, about 0.5 mg to about 200 mg, about 50 mg to about 500 mg, or about 150 mg to about 450 mg; or (ii) a dose of about 1 mg/kg to about 100 mg/kg, about 5 mg/kg to about 75 mg/kg, or about 25 mg/kg to about 75 mg/kg of methylnaltrexone or a salt thereof. In some embodiments, the pharmaceutical composition comprises: (i) about 25 mg to about 1000 mg, about 300 mg to about 750 mg, or about 500 mg to about 600 mg; or (ii) a dose of about 100 mg/kg to about 700 mg/kg, about 250 mg/kg to about 550 mg/kg or about 350 mg/kg to about 450 mg/kg of rifaximin.

### BRIEF DESCRIPTION OF THE DRAWINGS

FIG. 1A depicts a schematic of the study design used for testing the treatment of DIAMOND™ mouse model with methylnaltrexone (MNTX) and Rifaximin in Example 1.

FIG. 1B depicts the dextran ELISA results at week 12 and p-value matrix.

FIG. 1C depicts the dextran ELISA results at week 20 and p-value matrix. “X” indicates mean value and “O” indicates individual values.

FIG. 1D depicts the pair-wise comparison of dextran ELISA data from week 12-20, with p-value matrix. “X” indicates mean value and “O” indicates individual values.

FIG. 2A depicts a box plot of serum dextran concentrations at week 12 (4 weeks of treatment). “X” indicates mean value and “O” indicates individual values.

FIG. 2B depicts a box plot of serum dextran concentrations at week 20 (12 weeks of treatment). “X” indicates mean value and “O” indicates individual values.

FIG. 2C depicts a comparison of mean serum dextran concentration at weeks 12 and week 20.

FIG. 3A depicts 12-week dextran assay/gut permeability data. Box and whiskers plot (top panel), bar graph (middle panel) and basic descriptive statistics (bottom panel). “X” indicates mean value and “O” indicates individual values.

FIG. 3B depicts 12-week dextran assay/gut permeability data. p-value comparison matrix (top panel, two-tail, unequal variance T-test) and individual mouse data (bottom panel).

FIG. 3C depicts 20-week dextran assay/gut permeability data. Box and whiskers plot (top panel), bar graph (middle panel) and basic descriptive statistics (bottom panel). “X” indicates mean value and “O” indicates individual values.

FIG. 3D depicts 20-week dextran assay/gut permeability data. p-value comparison matrix (top panel, two-tail, unequal variance T-test) and individual mouse data (bottom panel).

FIG. 3E depicts comparison between week-12 and week-20 dextran assay/gut permeability for those mice with dextran assay data at both time points. Bar Graph (upper panel) and basic descriptive statistics and paired t-test (bottom panel).

FIG. 3F depicts week-12 and week-20 individual dextran assay/gut permeability data.

FIG. 4A depicts the experimental design used for evaluating the effects of methylnaltrexone, rifaximin, and rifaximin in combination with methylnaltrexone bromide (MNTX) on inflammation, NASH, and progression of fibrosis and gut permeability, in the DIAMOND™ (diet induced animal model of non-alcoholic fatty liver disease) mice.

FIG. 4B depicts the outline of the study by week.

FIG. 5A depicts animal body weight for study arms/groups 1, 2, 4, 9 and 10 throughout the duration of the study (in weeks).

FIG. 5B depicts animal body weight for study arms/groups 3, 5, 6, 7 and 8 throughout the duration of the study (in weeks).

FIG. 5C depicts body weight at necropsy. Box and whiskers plot (top panel), bar graph (middle panel) and basic descriptive statistics (bottom panel). “X” indicates mean value and “O” indicates individual values.

FIG. 5D depicts body weight at necropsy. p-value comparison matrix (top panel, two-tail, unequal variance T-test) and individual mouse data (bottom panel).

FIG. 6A depicts liver weight at necropsy. Box and whiskers plot (top panel), bar Graph (middle panel) and basic descriptive statistics (bottom panel). “X” indicates mean value and “O” indicates individual values.

FIG. 6B depicts liver weight at necropsy. p-value comparison matrix (top panel, two-tail, unequal variance T-test) and individual mouse data (bottom panel).

FIG. 7A depicts glucose blood levels. Box and whiskers plot (top panel), bar graph (middle panel) and basic descriptive statistics (bottom panel). “X” indicates mean value and “O” indicates individual values.

FIG. 7B depicts glucose blood level. p-value comparison matrix (top panel, two-tail, unequal variance T-test) and individual mouse data (bottom panel).

FIG. 8A depicts ketones blood levels. Box and whiskers plot (top panel), bar graph (middle panel) and basic descriptive statistics (bottom panel). “X” indicates mean value and “O” indicates individual values.

FIG. 8B depicts ketones blood level. p-value comparison matrix (top panel, two-tail, unequal variance T-test) and individual mouse data (bottom panel).

FIG. 9A depicts cholesterol blood levels. Box and whiskers plot (top panel), bar graph (middle panel) and basic descriptive statistics (bottom panel). “X” indicates mean value and “O” indicates individual values.

FIG. 9B depicts cholesterol blood level. p-value comparison matrix (top panel, two-tail, unequal variance T-test) and individual mouse data (bottom panel).

FIG. 10A depicts triglycerides blood levels. Box and whiskers plot (top panel), bar graph (middle panel) and basic descriptive statistics (bottom panel). “X” indicates mean value and “O” indicates individual values.

FIG. 10B depicts triglycerides blood level. p-value comparison matrix (top panel, two-tail, unequal variance T-test) and individual mouse data (bottom panel).

FIG. 11A depicts alanine aminotransferase (ALT) blood levels. Box and whiskers plot (top panel), bar graph (middle panel) and basic descriptive statistics (bottom panel). “X” indicates mean value and “O” indicates individual values.

FIG. 11B depicts ALT blood level. p-value comparison matrix (top panel, two-tail, unequal variance T-test) and individual mouse data (bottom panel).

FIG. 12A depicts alkaline phosphatase (ALP) blood levels. Box and whiskers plot (top panel), Bar Graph (middle panel) and basic descriptive statistics (bottom panel). “X” indicates mean value and “O” indicates individual values.

FIG. 12B depicts ALP blood level. p-value comparison matrix (top panel, two-tail, unequal variance T-test) and individual mouse data (bottom panel).

FIG. 13A depicts aspartate aminotransferase (AST) blood levels. Box and whiskers plot (top panel), Bar Graph (middle panel) and basic descriptive statistics (bottom panel). “X” indicates mean value and “O” indicates individual values.

FIG. 13B depicts AST blood level. p-value comparison matrix (top panel, two-tail, unequal variance T-test) and individual mouse data (bottom panel).

FIG. 14A depicts albumin blood levels. Box and whiskers plot (top panel), Bar Graph (middle panel) and basic descriptive statistics (bottom panel). “X” indicates mean value and “O” indicates individual values.

FIG. 14B depicts albumin blood level. p-value comparison matrix (top panel, two-tail, unequal variance T-test) and individual mouse data (bottom panel).

FIG. 15A depicts blood urea nitrogen levels. Box and whiskers plot (top panel), Bar Graph (middle panel) and basic descriptive statistics (bottom panel). “X” indicates mean value and “O” indicates individual values.

FIG. 15B depicts blood urea nitrogen level. p-value comparison matrix (top panel, two-tail, unequal variance T-test) and individual mouse data (bottom panel).

FIG. 16A depicts liver steatosis percentage. Box and whiskers plot (top panel), Bar Graph (middle panel) and basic descriptive statistics (bottom panel). “X” indicates mean value and “O” indicates individual values.

FIG. 16B depicts liver steatosis percentage. p-value comparison matrix (top panel, two-tail, unequal variance T-test) and individual mouse data (bottom panel).

FIG. 17A depicts liver steatosis grade. Box and whiskers plot (top panel), Bar Graph (middle panel) and basic descriptive statistics (bottom panel). “X” indicates mean value and “O” indicates individual values.

FIG. 17B depicts liver steatosis grade. p-value comparison matrix (top panel, two-tail, unequal variance T-test) and individual mouse data (bottom panel).

FIG. 18A depicts hepatocyte ballooning. Box and whiskers plot (top panel), Bar Graph (middle panel) and basic descriptive statistics (bottom panel). “X” indicates mean value and “O” indicates individual values.

FIG. 18B depicts hepatocyte ballooning. p-value comparison matrix (top panel, two-tail, unequal variance T-test) and individual mouse data (bottom panel).

FIG. 19A depicts lobular inflammation. Box and whiskers plot (top panel), Bar Graph (middle panel) and basic descriptive statistics (bottom panel). “X” indicates mean value and “O” indicates individual values.

FIG. 19B depicts lobular inflammation. p-value comparison matrix (top panel, two-tail, unequal variance T-test) and individual mouse data (bottom panel).

FIG. 20A depicts the NAFLD activity score (NAS). Box and whiskers plot (top panel), bar graph (middle panel) and basic descriptive statistics (bottom panel). “X” indicates mean value and “O” indicates individual values.

FIG. 20B depicts the NAS p-value comparison matrix (top panel, two-tail, unequal variance T-test) and individual mouse data (bottom panel).

FIG. 21A depicts the steatosis, activity, and fibrosis (SAF) score. Box and whiskers plot (top panel), bar graph (middle panel) and basic descriptive statistics (bottom panel). “X” indicates mean value and “O” indicates individual values.

FIG. 21B depicts the SAF score. p-value comparison matrix (top panel, two-tail, unequal variance T-test) and individual mouse data (bottom panel).

FIG. 22A depicts liver fibrosis. Box and whiskers plot (top panel), bar graph (middle panel) and basic descriptive statistics (bottom panel). “X” indicates mean value and “O” indicates individual values.

FIG. 22B depicts liver fibrosis. p-value comparison matrix (top panel, two-tail, unequal variance T-test) and individual mouse data (bottom panel).

FIG. 23A depicts perisinusoidal fibrosis. Box and whiskers plot (top panel), Bar Graph (middle panel) and basic descriptive statistics (bottom panel). “X” indicates mean value and “O” indicates individual values.

FIG. 23B depicts perisinusoidal fibrosis. p-value comparison matrix (top panel, two-tail, unequal variance T-test) and individual mouse data (bottom panel).

FIG. 24A depicts the percentage of disease categories in each group at end of study. (0=Normal histology, 1=Steatosis, 2=NASH).

FIG. 24B depicts the percentage of disease categories in each group at end of study and individual disease category classification for each mouse in each group (0=Normal histology, 1=Steatosis, 2=NASH).

### DETAILED DESCRIPTION

The invention described herein is predicated, at least in part, on the surprising discovery that the μ-opioid receptor antagonist methylnaltrexone bromide and the antibiotic rifaximin are effective in the treatment of increased gut permeability (i.e., leaky gut) and, as a result, can serve to treat related diseases such as the fatty liver diseases nonalcoholic steatohepatitis (NASH) and nonalcoholic fatty liver disease (NAFLD), in addition to other diseases and conditions described herein. While methylnaltrexone bromide and rifaximin have previously been used as effective treatments for other diseases, the finding that such agents can be used to treat increased gut permeability or leaky gut and certain related diseases is surprising.

Accordingly, in one aspect, the invention is directed to a method of treating increased gut permeability in a subject in need thereof, by administering a therapeutically effective amount of at least one therapeutic agent selected from the group consisting of methylnaltrexone, or a salt thereof and rifaximin. In another aspect, the invention is directed to a method of reducing gut permeability in a subject in need thereof, by administering a therapeutically effective amount of a therapeutic agent selected from the group consisting of methylnaltrexone, or a salt thereof, rifaximin, and a combination thereof. In another aspect, the invention is directed to a method of treating a disease or condition associated with increased gut permeability in a subject in need thereof, by administering a therapeutically effective amount of a therapeutic agent selected from the group consisting of methylnaltrexone, or a salt thereof, rifaximin, and a combination thereof. In yet another aspect, the invention is directed to a method of treating NASH or a symptom thereof in a subject in need thereof, by administering a therapeutically effective amount of a therapeutic agent selected from the group consisting of methylnaltrexone, or a salt thereof, rifaximin, and a combination thereof. In some embodiments, the disease or condition associated with increased gut permeability is selected from the group consisting of irritable bowel syndrome, inflammatory bowel disease, Crohn's disease, ulcerative colitis, indeterminate colitis, chemotherapy-induced colitis, celiac disease, infectious diarrhea, atopy, allergy, food allergy, asthma, autism, chronic fatigue syndrome, lupus, metabolic syndromes, diabetes, type 1 diabetes, type 2 diabetes, hypertension, hyperlipidemia, neoplasia, cancer, idiopathic inflammatory conditions, rheumatoid arthritis, neurologic disorders, multiple sclerosis, migraines, psoriatic arthritic, autoimmune diseases, rheumatoid arthritis, skin disorders, psoriasis, eczema, acne, metabolic bone disease, osteoporosis in adults, primary growth failure in children, liver disease, fatty liver diseases, nonalcoholic steatohepatitis (NASH), nonalcoholic fatty liver disease (NAFLD), hepatic encephalopathy (HE), anklyosing spondylitis, obesity, graft versus host disease (GVHD), multiple organ dysfunction syndrome, Parkinson's disease, acute pancreatitis, fibromyalgia, mental illness, depression, schizophrenia, burn injury, heart failure, and renal failure. In some embodiments, the disease or condition associated with increased gut permeability is a liver disease, optionally, a fatty liver disease, optionally, selected from the group consisting of NASH and NAFLD. In some embodiments, the disease or condition associated with increased gut permeability is hepatic encephalopathy (HE). In yet another aspect, the invention is directed to a method of treating NASH or a symptom thereof in a subject in need thereof, by administering a therapeutically effective amount of a therapeutic agent selected from the group consisting of methylnaltrexone, or a salt thereof, rifaximin, and a combination thereof. In yet another aspect, the invention is directed to a method of treating NAFLD or a symptom thereof in a subject in need thereof, by administering a therapeutically effective amount of a therapeutic agent selected from the group consisting of methylnaltrexone, or a salt thereof, rifaximin, and a combination thereof. In yet another aspect, the invention is directed to a method of treating hepatic encephalopathy (HE) or a symptom thereof in a subject in need thereof, by administering a therapeutically effective amount of (i) a therapeutically effective amount of methylnaltrexone, or a salt thereof; or (ii) a therapeutically effective amount of methylnaltrexone, or a salt thereof, and rifaximin.

In other aspects, the invention includes a pharmaceutical composition for treating increased gut permeability, for reducing gut permeability, for treating NASH or a symptom thereof, or for treating NAFLD or a symptom thereof in a subject in need thereof. In other aspects, the invention includes a pharmaceutical composition for treating increased gut permeability, for reducing gut permeability, for treating HE or a symptom thereof in a subject in need thereof. In some embodiments, the pharmaceutical composition may include a therapeutically effective amount of methylnaltrexone or a salt thereof, a therapeutically effective amount of rifaximin, and a pharmaceutically acceptable carrier.

### Definitions

Unless otherwise defined herein, scientific and technical terms used herein shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including,” as well as other forms of the term, such as “includes” and “included”, is not limiting.

As used herein, an “effective amount” of a composition of methylnaltrexone, or a salt thereof, rifaximin, or a combination thereof refers to the level required to treat or prevent one or more symptoms of increased gut permeability, or an associated disorder, e.g., NASH, NAFLD, or HE. In some embodiments, an “effective amount” is at least a minimal amount of methylnaltrexone, or a salt thereof, rifaximin, or a combination thereof, which is sufficient for treating or preventing one or more symptoms of increased gut permeability, or an associated disorder, e.g., NASH, NAFLD, or HE, as defined herein. In some embodiments, the term “effective amount,” as used in connection with an amount of methylnaltrexone, salt thereof; rifaximin, salt thereof; or combinations thereof, refers to an amount of methylnaltrexone, salt thereof; rifaximin, salt thereof; or combinations thereof sufficient to treat or prevent one or more symptoms of increased gut permeability, or an associated disorder, e.g., NASH, NAFLD, or HE, in a subject. An effective amount of a composition of methylnaltrexone, rifaximin, or a combination thereof may vary according to factors such as the disease state, age, and weight of the subject, and the ability of a composition of methylnaltrexone, or a salt thereof, rifaximin, or a combination thereof to elicit a desired response in the subject. Dosage regimens may be adjusted to provide the optimum therapeutic response. An effective amount is also one in which any toxic or detrimental effects (e.g., side effects) of a composition of methylnaltrexone, or a salt thereof, rifaximin, or a combination thereof are outweighed by the therapeutically beneficial effects.

The terms “treat” or “treating,” as used herein, refer to partially or completely alleviating, inhibiting, delaying onset of, reducing the incidence of, ameliorating and/or relieving increased gut permeability, or an associated disorder, e.g., NASH, NAFLD, or HE, or one or more symptoms of increased gut permeability, or an associated disorder, e.g., NASH, NAFLD, or HE. In some embodiments, the terms “treat” or “treating” refers to partially or completely alleviating, inhibiting, delaying onset of, reducing the incidence of, ameliorating and/or relieving increased gut permeability, or an associated disorder, selected from the group consisting of irritable bowel syndrome, inflammatory bowel disease, Crohn's disease, ulcerative colitis, indeterminate colitis, chemotherapy-induced colitis, celiac disease, infectious diarrhea, atopy, allergy, food allergy, asthma, autism, chronic fatigue syndrome, lupus, metabolic syndromes, diabetes, type 1 diabetes, type 2 diabetes, hypertension, hyperlipidemia, neoplasia, cancer, idiopathic inflammatory conditions, rheumatoid arthritis, neurologic disorders, multiple sclerosis, migraines, psoriatic arthritic, autoimmune diseases, rheumatoid arthritis, skin disorders, psoriasis, eczema, acne, metabolic bone disease, osteoporosis in adults, primary growth failure in children, liver disease, fatty liver diseases, nonalcoholic steatohepatitis (NASH), nonalcoholic fatty liver disease (NAFLD), hepatic encephalopathy (HE), anklyosing spondylitis, obesity, graft versus host disease (GVHD), multiple organ dysfunction syndrome, Parkinson's disease, acute pancreatitis, fibromyalgia, mental illness, depression, schizophrenia, burn injury, heart failure, and renal failure, e.g., using a pharmaceutical composition or method described herein.

The expression “unit dosage form” as used herein refers to a physically discrete unit of a composition or formulation of methylnaltrexone, or a salt thereof, rifaximin, or a combination thereof, appropriate for the subject to be treated. The specific effective dose level for any particular subject will depend upon a variety of factors including the severity of the increased gut permeability, or an associated disorder, e.g., NASH, NAFLD, or HE; nature and activity of the composition; specific formulation employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

The term “subject”, as used herein, means a mammal and includes human and animal subjects, such as domesticated animals (e.g., horses, dogs, cats, etc.) and experimental animals (e.g., mice, rats, dogs, chimpanzees, apes, etc.). In a particular embodiment, the subject is a human.

The terms “suffer” or “suffering,” as used herein, refer to one or more conditions that a subject has been diagnosed with, or is suspected to have, in particular, increased gut permeability, or an associated disorder, e.g., NASH, NAFLD, or HE.

The term “amphiphilic,” as used herein, to describe a molecule refers to the molecule's dual hydrophobic and hydrophilic properties. Typically, amphiphilic molecules have a polar, water soluble group (e.g., a phosphate, carboxylic acid, sulfate) attached to a nonpolar, water-insoluble group (e.g., a hydrocarbon). The term amphiphilic is synonymous with amphipathic. Examples of amphiphilic molecules include sodium dodecyl (lauryl) sulfate, fatty acids, phospholipids, and bile acids. Amphiphilic molecules may be uncharged, cationic, or anionic.

As used herein, the term “lipophilicity” refers to a compound's ability to associate with or dissolve in a fat, lipid, oil, or non-polar solvent. Lipophilicity and hydrophobicity may be used to describe the same tendency of a molecule to dissolve in fats, oils, lipids, and non-polar solvents.

The terms “administration” or “administering” include routes of introducing a composition(s) of methylnaltrexone, rifaximin, or a combination thereof to a subject to perform their intended function. In embodiments where both methylnaltrexone, or a salt thereof, and rifaximin are administered, they may be administered as distinct compositions, whether concurrently or separately, or administered as formulation that includes both methylnaltrexone, or a salt thereof, and rifaximin. As used herein, administration of composition(s) of methylnaltrexone, or a salt thereof, rifaximin, or combinations thereof are intended to include injection (subcutaneous, intravenous, parenterally, intraperitoneally, intrathecal), oral or subcutaneous, rectal, and transdermal. The pharmaceutical preparations may be given by forms suitable for each administration route. For example, these preparations are administered in tablet or capsule form, ointment, suppository, and administration by injection or infusion; topical by lotion or ointment; and rectal by suppositories. Oral or subcutaneous administration is preferred. The injection can be bolus or can be continuous infusion. Depending on the route of administration, a composition(s) of methylnaltrexone, or a salt thereof, rifaximin, or a combination thereof can be coated with or disposed in a selected material to protect it from natural conditions that may detrimentally affect its ability to perform its intended function. A composition(s) of methylnaltrexone, or a salt thereof, rifaximin, or a combination thereof can be administered alone, or in conjunction with either another agent or agents as described above or with a pharmaceutically-acceptable carrier, or both. A composition(s) of methylnaltrexone, or a salt thereof, rifaximin, or a combination thereof can be administered prior to the administration of the other agent, simultaneously with the agent, or after the administration of the agent.

In certain embodiments, the subject is administered the pharmaceutical composition as described herein at least once a day. In certain embodiments, the subject is administered the pharmaceutical composition as described herein at least twice a day. In certain embodiments, the subject is administered the pharmaceutical composition as described herein at least three times a day. In other embodiments, the subject is administered the pharmaceutical composition up to once a day. In other embodiments, the subject is administered the pharmaceutical composition up to twice a day. In other embodiments, the subject is administered the pharmaceutical composition up to three times a day. In certain embodiments, the subject is administered the pharmaceutical composition not more than once a day. In certain embodiments, the subject is administered the pharmaceutical composition not more than twice a day. In certain embodiments, the subject is administered the pharmaceutical composition not more than three times a day. In certain embodiments, the subject is administered the pharmaceutical composition as needed. In certain embodiments, the subject is administered the pharmaceutical composition as needed, but not more than once a day. In certain embodiments, the subject is administered the pharmaceutical composition as needed, but not more than twice a day. In certain embodiments, the subject is administered the pharmaceutical composition as needed, but not more than three times a day.

Administration “in combination with” one or more further therapeutic agents include simultaneous (concurrent) and consecutive administration in any order.

As will be readily apparent to one skilled in the art, the useful in vivo dosage to be administered and the particular mode of administration will vary depending upon the age, weight and mammalian species treated, the particular compounds employed, and the specific use for which these compounds are employed. Typically, human clinical applications of products are commenced at lower dosage levels, with dosage level being increased until the desired effect is achieved.

As used herein, an “increase” or “decrease” in a measurement, unless otherwise specified, is typically in comparison to a baseline value. For example, an increase or decrease in gut permeability may be in comparison to a baseline level of gut permeability expected for healthy subjects. Alternatively, an increase or decrease in gut permeability may be in comparison to a previous time point for the same subject, for example, prior to treatment. In some instances, an increase or decrease in a measurement can be evaluated based on the context in which the term is used.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG).

“Ameliorate,” “amelioration,” “improvement” or the like refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change may be observed in treated subjects as compared to subjects not treated with methylnaltrexone, or a salt thereof, rifaximin, or a combination thereof, where the untreated subjects have, or are subject to developing, the same or similar disease, condition, symptom or the like. Amelioration of a disease, condition, symptom or assay parameter may be determined subjectively or objectively, e.g., self-assessment by a subject(s), by a clinician's assessment or by conducting an appropriate assay or measurement. Amelioration may be transient, prolonged or permanent or it may be variable at relevant times during or after a composition of methylnaltrexone, rifaximin, or a combination thereof is administered to a subject.

### Therapeutic Agents

In an embodiment, the pharmaceutical compositions and methods of treatment described herein include one or more active agents. Methylnaltrexone or a salt thereof may be an active agent provided at an amount and/or dosage described herein in the pharmaceutical compositions and methods of treatment of the invention. Rifaximin may be an active agent provided in an amount and/or dosage described herein in the pharmaceutical compositions and methods of treatment of the invention.

### Methylnaltrexone

In some embodiments, the pharmaceutical compositions and methods described herein may include methylnaltrexone or a salt thereof in a therapeutically effective amount. In some embodiments, the pharmaceutical compositions and methods described herein may include methylnaltrexone or a salt thereof in an amount of about 1 to about 1000 mg of methylnaltrexone or a salt thereof. In some embodiments, the pharmaceutical compositions and methods described may include methylnaltrexone or a salt thereof in an amount of about 0.5 mg to about 500 mg, or about 0.5 mg to about 200 mg, or about 50 mg to about 500 mg, or about 150 mg to about 450 mg, or about 10 mg to about 600 mg, or about 75 mg to about 900 mg. In some embodiments, the pharmaceutical composition and methods described herein may include methylnaltrexone or a salt thereof in an amount of at least about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 mg.

In some embodiments, the pharmaceutical composition and methods described herein may include methylnaltrexone or a salt thereof in an amount of at most about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 mg.

In some embodiments, the pharmaceutical composition and methods described herein may include methylnaltrexone or a salt thereof in an amount of about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 mg.

In some embodiments, the pharmaceutical composition and methods described herein may include methylnaltrexone or a salt thereof in an amount to provide a dosage to the subject of about 0.01 mg/kg to about 80 mg/kg, about 1 mg/kg to about 100 mg/kg, or about 5 mg/kg to about 75 mg/kg, or about 25 mg/kg to about 75 mg/kg.

In some embodiments, the pharmaceutical composition and methods described herein may include methylnaltrexone or a salt thereof in an amount to provide a dosage to the subject of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

In some embodiments, the pharmaceutical composition and methods described herein may include methylnaltrexone or a salt thereof in an amount to provide a dosage to the subject of at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

In some embodiments, the pharmaceutical composition and methods described herein may include methylnaltrexone or a salt thereof in an amount to provide a dosage to the subject of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

In some embodiments, methylnaltrexone provided in the pharmaceutical composition and methods described herein may include methylnaltrexone and an amphiphilic pharmaceutically acceptable excipient. For example, the methylnaltrexone may be a salt of methylnaltrexone of the formula:

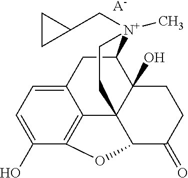

wherein methylnaltrexone is the cation of the salt, and A⁻ is an anion of an amphiphilic pharmaceutically acceptable excipient, as described in U.S. Pat. No. 8,524,276, the entire contents of which are hereby incorporated by reference herein. In certain embodiments, the methylnaltrexone is (R)-N-methylnaltrexone, a peripherally acting μ opioid receptor antagonist, as shown in the formula above. It will be understood that the (R)-N-methylnaltrexone cation and the anion of the amphiphilic pharmaceutically acceptable excipient may exist in the composition as an ion pair or may exist as separate salts paired with other counter ions. For example, methylnaltrexone included in the pharmaceutical compositions and methods of the invention described herein may be provided as (R)-N-methylnaltrexone bromide and sodium lauryl sulfate.

As set forth herein, pharmaceutical compositions and methods of the invention may include an amphiphilic pharmaceutically acceptable excipient or an anion thereof (A⁻). The amphiphilic pharmaceutically acceptable excipient increases the lipophilicity of the composition thereby allowing for increased transport through the unstirred diffusion layer in the GI tract, resulting in increased permeation through biological membranes. In certain embodiments, the excipient increases the lipophilicity of a methylnaltrexone.

In certain embodiments, the amphiphilic pharmaceutically acceptable excipient may include a sulfate, sulfonate, nitrate, nitrite, phosphate, or phosphonate moiety. In one embodiment, the pharmaceutically acceptable excipient comprises an (—OSO₃⁻) group. In certain embodiments, the anion is butyl sulfate, pentyl sulfate, hexyl sulfate, heptyl sulfate, octyl sulfate, nonyl sulfate, decyl sulfate, undecyl sulfate, dodecyl sulfate, tridecyl sulphate, tetradecyl sulfate, pentadecyl sulfate, hexadecyl sulfate, heptadecyl sulfate, octadecyl sulfate, eicosyl sulfate, docosyl sulfate, tetracosyl sulfate, hexacosyl sulfate, octacosyl sulfate, and triacontyl sulphate.

In certain embodiments, A⁻ is the anion of a Brønsted acid. Exemplary Brønsted acids include hydrogen halides, carboxylic acids, sulfonic acids, sulfuric acid, and phosphoric acid. In certain embodiments, A⁻ is chloride, bromide, iodide, fluoride, sulfate, bisulfate, tartrate, nitrate, citrate, bitartrate, carbonate, phosphate, malate, maleate, fumarate sulfonate, methylsulfonate, formate, carboxylate, sulfate, methylsulfate or succinate salt. In certain embodiments, A⁻ is trifluoroacetate or bromide.

In certain embodiments, the methylnaltrexone of the pharmaceutical compositions and methods described herein may have multiple anions (e.g., bromide and dodecyl (lauryl) sulfate) associating therewith.

In certain embodiments, A⁻ is bromide, such that methylnaltrexone may be (R)-N-methylnaltrexone bromide. (R)-N-methylnaltrexone bromide, which is also known as “MNTX” and is described in U.S. Pat. No. 8,343,992, which is incorporated herein by reference. The chemical name for (R)-N-methylnaltrexone bromide is (R)-N-(cyclopropylmethyl) noroxymorphone methobromide. (R)-N-methylnaltrexone bromide has the molecular formula C₂₁H₂₆NO₄Br and a molecular weight of 436.36 g/mol. (R)-N-methylnaltrexone bromide has the following structure:

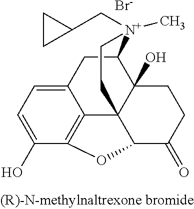 [SMILES: [Br-] ; C[N+]1(CC2CC2)CC[C@]23c4c5ccc(O)c4O[C@H]2C(=O)CC[C@@]3(O)[C@H]1C5]

where the compound is in the (R) configuration with respect to the quaternary nitrogen. In certain embodiments presented herein, at least about 99.6%, 99.7%, 99.8%, 99.85%, 99.9%, or 99.95% of the compound is in the (R) configuration with respect to nitrogen. Methods for determining the amount of (R)-N-methylnaltrexone bromide, present in a sample as compared to the amount of (S)-N-methylnaltrexone bromide present in that same sample, are described in U.S. Pat. No. 8,343,992, which is incorporated herein by reference. In other embodiments, the methylnaltrexone contains 0.15%, 0.10%, or less (S)-N-methylnaltrexone bromide.

In certain embodiments, A⁻ is an acidic amphiphilic pharmaceutically acceptable excipient. In certain embodiments, the pharmaceutically acceptable excipient has a pKₐ of about 3 or less. In certain embodiments, the pharmaceutically acceptable excipient has a pKₐ of about 2 or less. In certain embodiments, the pharmaceutically acceptable excipient has a pKₐ between about 1 and about 2. In certain embodiments, the pharmaceutically acceptable excipient has a pKₐ of about 1 or less.

In some embodiments, methylnaltrexone for use in pharmaceutical compositions and methods described herein may be provided in any of a variety of forms. For example, forms of methylnaltrexone suitable for use herein include pharmaceutically acceptable salts, prodrugs, polymorphs (i.e., crystal forms), co-crystals, hydrates, solvates, and the like. In some embodiments, the form of methylnaltrexone used should allow for association with the amphiphilic pharmaceutically acceptable excipient, for example, through ion pairing either in the pharmaceutical composition or in situ.

In some embodiments, pharmaceutical compositions and methods described herein that include oral delivery of methylnaltrexone may include an amphiphilic pharmaceutically acceptable excipient as described above, and a disintegrant, and further, optionally, comprise one or more other components, such as, for example, binders, carriers, chelating agents, antioxidants, fillers, lubricants, wetting agents, or combinations thereof, as set forth in U.S. Pat. No. 9,314,461, the entire contents of which are incorporated herein by reference.

In some embodiments, the pharmaceutical compositions and methods described herein includes a combination of methylnaltrexone bromide and sodium dodecyl (lauryl) sulfate (also known as SDS or SLS). In certain embodiments, the pharmaceutical compositions and methods described herein may include delivery of sodium bicarbonate as a disintegrant. Additional excipients, as set forth above, may be incorporated, including, but not limited to, at least one of microcrystalline cellulose, crospovidone, polysorbate 80, edetate calcium disodium dehydrate, silicified microcrystalline cellulose, talc, colloidal silicon dioxide and magnesium stearate. In one embodiment, the pharmaceutical compositions and methods described herein may include delivery of methylnaltrexone bromide, sodium lauryl sulfate, sodium bicarbonate, microcrystalline cellulose, crospovidone, polysorbate 80, edetate calcium disodium dehydrate, silicified microcrystalline cellulose, talc, colloidal silicon dioxide and magnesium stearate.

In some embodiments, methylnaltrexone is provided in the methods described herein as oral RELISTOR, an FDA approved tablet product for oral administration. In some embodiments, methylnaltrexone is provided in the methods described herein as one or more tablets of oral RELISTOR (150 mg).

In some embodiments, the methods described herein include the parenteral administration of methylnaltrexone, for example, methylnaltrexone bromide, as, for example, a subcutaneous methylnaltrexone formulation. In some embodiments, a subcutaneous methylnaltrexone formulation may include methylnaltrexone, for example, methylnaltrexone bromide, a chelating agent such as a calcium salt chelating agent, a stabilizing agent, an isotonic agent, and a carrier. In some embodiments, the pH of the formulation is between about a pH of 2 to about a pH of 6.

In some embodiments, subcutaneous methylnaltrexone formulations may include methylnaltrexone, for example, methylnaltrexone bromide, together with one or more excipients, such as, for example, one or more chelating agents, a calcium ion, isotonic agents, carriers, buffers, co-solvents, diluents, preservatives, and/or surfactants, or combinations thereof. One skilled in the art will readily appreciate that the same ingredient can sometimes perform more than one function, or can perform different functions in the context of different formulations, and/or portions of a formulation, depending upon the amount of the ingredient and/or the presence of other ingredients and/or active compound(s).

In some embodiments, methylnaltrexone is provided in the methods described herein as subcutaneous RELISTOR. In some embodiments, methylnaltrexone is provided in the methods described herein as one or more doses of subcutaneous RELISTOR (8 mg/0.4 mL) or one or more doses of subcutaneous RELISTOR (12 mg/0.6 mL).

In some embodiments, the subcutaneous methylnaltrexone, for example, methylnaltrexone bromide, formulation comprises a chelating agent. In some embodiments, a chelating agent may be present in an amount from about 0.01 mg/mL to about 2 mg/mL or about 0.1 mg/mL to about 1 mg/mL in the formulation, or about 0.2 mg/mL to about 0.8 mg/mL of the formulation. In some embodiments, a chelating agent may be present in an amount from about 0.2 mg/mL, about 0.3 mg/mL, about 0.4 mg/mL, about 0.5 mg/mL, or about 0.6 mg/mL, in the formulation.

Examples of chelating agents include, but are not limited to, ethylenediaminetetraacetic acid (also synonymous with EDTA, edetic acid, versene acid, and sequestrene), and EDTA derivatives, such as sodium EDTA, and potassium EDTA, diammonium EDTA, dipotassium EDTA, disodium EDTA, TEA-EDTA, tetrasodium EDTA, tripotassium EDTA, trisodium EDTA, HEDTA, and trisodium HEDTA, and related salts thereof. Other chelating agents include niacinamide and derivatives thereof and sodium desoxycholate and derivatives thereof, ethylene glycol-bis-(2-aminoethyl)-N,N,N′, N′-tetraacetic acid (EGTA) and derivatives thereof, diethylenetriaminepentaacetic acid (DTPA) and derivatives thereof, N,N-bis(carboxymethyl)glycine (NTA) and derivatives thereof, nitrilotriacetic acid and derivatives thereof. Still other chelating agents include citric acid and derivatives thereof. Citric acid also is known as citric acid monohydrate. Derivatives of citric acid include anhydrous citric acid and trisodiumcitrate-dihydrate. In some embodiments, chelating agent is selected from EDTA or an EDTA derivative or EGTA or an EGTA derivative. In some embodiments chelating agent is EDTA disodium such as, for example, EDTA disodium hydrate.

In some embodiments, a subcutaneous methylnaltrexone, for example, methylnaltrexone bromide, formulation comprises a calcium ion and a chelating agent included as a single component of the formulation. Thus, in some embodiments a calcium salt chelating agent may be present in an amount from about 0.01 mg/mL to about 2 mg/mL or about 0.1 mg/mL to about 1 mg/mL in the formulation, or about 0.2 mg/mL to about 0.8 mg/mL of the formulation. In some embodiments, calcium salt chelating agent may be present in an amount from about 0.2 mg/mL, about 0.3 mg/mL, about 0.4 mg/mL, about 0.5 mg/mL, or about 0.6 mg/mL, in the formulation.

Appropriate calcium salt chelating agents include any pharmaceutically acceptable chelating agents and calcium salts thereof. Common calcium salt chelating agents include, but are not limited to calcium ethylenediaminetetraacetic acid (EDTA) and calcium salt EDTA derivatives, calcium ethylene glycol-bis-(2-aminoethyl)-N,N,N′, N′-tetraacetic acid (EGTA) and calcium salt EGTA derivatives, calcium diethylenetriaminepentaacetic acid (DTPA) and calcium salt DTPA derivatives, calcium N,N-bis(carboxymethyl)glycine (NTA) and calcium salt NTA derivatives, and calcium citrate and derivatives thereof. In some embodiments, chelating agent is selected from calcium EDTA or a calcium salt EDTA derivative or calcium EGTA or a calcium salt EGTA derivative. In some embodiments, the chelating agent may be calcium EDTA disodium such as, for example, calcium EDTA disodium hydrate.

In some embodiments, a subcutaneous methylnaltrexone formulation comprises at least methylnaltrexone, for example, methylnaltrexone bromide, a calcium salt chelating agent and an isotonic agent. An isotonic agent useful in such formulations can be any pharmaceutically acceptable isotonic agent. Common isotonic agents include agents selected from the group consisting of sodium chloride, mannitol, lactose, dextrose (hydrous or anhydrous), sucrose, glycerol, and sorbitol, and solutions of the foregoing. In certain embodiments, the formulation comprises methylnaltrexone bromide, an isotonic agent which is sodium chloride, and a calcium salt chelating agent which is calcium EDTA or a calcium salt EDTA derivative. In some embodiments, the EDTA is calcium EDTA disodium.

Still additional components such as stabilizing agents, buffers, co-solvents, diluents, preservatives, and/or surfactants, and the like, may be included in subcutaneous methylnaltrexone formulations. In some embodiments, such formulations may contain additional agents which comprise from about 1% to about 30% or about 1% to about 12% of the formulation or about 1% to about 10%, based upon total weight of the formulation. In some embodiments, additional agents may comprise from about 1%, about 2%, about 5%, about 8% or about 10% of the formulation, based upon total weight of the formulation. Optionally included additional ingredients are described below.

In some embodiments, subcutaneous methylnaltrexone, for example, methylnaltrexone bromide, formulations comprise a stabilizing agent. In some embodiments, stabilizing agent may be present in an amount from about 0.01 mg/mL to about 2 mg/mL or about 0.05 mg/mL to about 1 mg/mL in the formulation, or about 0.1 mg/mL to about 0.8 mg/mL in the formulation. In some embodiments, stabilizing agent may be present in an amount from about 0.15 mg/mL, about 0.2 mg/mL, about 0.25 mg/mL, about 0.3 mg/mL, about 0.35 mg/mL, or about 0.4 mg/mL.

Suitable stabilizing agents for use in the subcutaneous methylnaltrexone formulations described herein include, but are not limited to glycine, benzoic acid, citric, glycolic, lactic, malic, and maleic acid. In some embodiments, such formulations comprise glycine. In some embodiments, glycine comprises glycine-HCl.

In certain embodiments, a stabilizing agent is added to the subcutaneous methylnaltrexone, for example, methylnaltrexone bromide, formulation in an amount sufficient to adjust and maintain the pH of the formulation. Thus, in some embodiments, a stabilizing agent acts as a buffer function in addition to its role as a stabilizer. In some embodiments, a stabilizing agent may act as a buffer agent, so as to maintain the pH of the formulation. In certain embodiments, the pH is between about pH 2.0 and about pH 6.0. In some embodiments, the pH of the formulation is between about pH 2.6 and about pH 5.0. In some embodiments, the pH of the formulation is between about pH 3.0 and about pH 4.0. In some embodiments, the pH of the formulation is between about pH 3.4 and about pH 3.6. In some embodiments, the pH of the formulation is about pH 3.5.

In some embodiments, subcutaneous methylnaltrexone formulations that may be used in the methods described herein may comprise methylnaltrexone bromide, calcium EDTA or a calcium salt EDTA derivative, water for injection, sodium chloride, glycine HCl, and the pH of the formulation is between about pH 3.0 and about pH 4.0. In some embodiments, such formulations comprise methylnaltrexone, for example, methylnaltrexone bromide, calcium EDTA or a calcium salt EDTA derivative, water for injection, sodium chloride, glycine HCl, and the pH of the formulation is between about pH 3.4 and about pH 3.6. In some embodiments, such formulations comprise methylnaltrexone bromide, calcium EDTA disodium, water for injection, sodium chloride, and glycine HCl, and the formulation has a pH of about 3.5. In certain embodiments, the pH is adjusted with glycine. In some embodiments, glycine is glycine HCl.

### Rifaximin

In some embodiments, the pharmaceutical compositions and methods described herein may include rifaximin in a therapeutically effective amount. In some embodiments, rifaximin may be provided in an amount of about 25 mg to about 1000 mg, or about 300 mg to about 750 mg, or about 500 mg to about 600 mg.

In some embodiments, the pharmaceutical composition and methods described herein may include rifaximin in an amount of at least about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 mg.

In some embodiments, the pharmaceutical composition and methods described herein may include rifaximin in an amount of at most about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 mg.

In some embodiments, the pharmaceutical composition and methods described herein may include rifaximin in an amount of about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 mg.

In some embodiments, the pharmaceutical composition and methods described herein may include rifaximin in an amount to provide a dosage to the subject of about 100 mg/kg to about 700 mg/kg, or about 250 mg/kg to about 550 mg/kg, or about 350 mg/kg to about 450 mg/kg.

In some embodiments, the pharmaceutical composition and methods described herein may include rifaximin in an amount to provide a dosage to the subject of at least about 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600 mg/kg.

In some embodiments, the pharmaceutical composition and methods described herein may include rifaximin in an amount to provide a dosage to the subject of at most about 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600 mg/kg.

In some embodiments, the pharmaceutical composition and methods described herein may include rifaximin in an amount to provide a dosage to the subject of about 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600 mg/kg.

As used with regard to the pharmaceutical compositions and methods described herein, “rifaximin” includes, without limitation, all solvate and polymorph forms of the molecule, including, for example, α, β, γ, δ, ε, η, ζ, and amorphous forms of rifaximin or combinations thereof. Forms, pharmaceutical compositions, and methods of using rifaximin are described, for example, in U.S. Pat. Nos. 7,045,620, 7,906,542, 7,915,275, 8,193,196, 8,309,569, 8,518,949, and 8,741,904, the entirety of which are incorporated herein by reference. Rifaximin is a nonaminoglycoside, semisynthetic antibiotic derived from rifamycin O. It is a non-systemic, non-absorbed, broad-spectrum, oral or subcutaneous antibiotic specific for enteric pathogens of the GI tract. Rifaximin was found to be advantageous in treatment of hepatic encephalopathy (HE) relative to previously used antibiotics; e.g., negligible systemic absorption (<0.4%) regardless of food intake or presence of GI disease and exhibits no plasma accumulation with high or repeat doses. The lack of systemic absorption makes rifaximin safe and well tolerated, thus improving subject compliance and reducing side effects associated with currently known treatments.

Rifaximin (INN; see The Merck Index, XIII Ed., 8304) is an antibiotic belonging to the rifamycin class of antibiotics, e.g., a pyrido-imidazo rifamycin. Rifaximin exerts its broad antibacterial activity, for example, in the gastrointestinal tract against localized gastrointestinal bacteria that cause infectious diarrhea, irritable bowel syndrome, small intestinal bacterial overgrowth, Crohn's disease, and/or pancreatic insufficiency. It has been reported that rifaximin is characterized by a negligible systemic absorption, due to its chemical and physical characteristics (Descombe J. J. et al. Pharmacokinetic study of rifaximin after oral or subcutaneous administration in healthy volunteers. Int J Clin Pharmacol Res, 14 (2), 51-56, (1994)).

Rifaximin exerts a broad antibacterial activity in the gastrointestinal tract against localized gastrointestinal bacteria that cause infectious diarrhea, including anaerobic strains. It has been reported that rifaximin is characterized by a negligible systemic absorption, due to its chemical and physical characteristics (Descombe J. J. et al. Pharmacokinetic study of rifaximin after oral or subcutaneous administration in healthy volunteers. Int J Clin Pharmacol Res, 14 (2), 51-56, (1994)).

Without wishing to be bound by any particular scientific theories, rifaximin acts by binding to the beta-subunit of the bacterial deoxyribonucleic acid-dependent ribonucleic acid (RNA) polymerase, resulting in inhibition of bacterial RNA synthesis. It is active against numerous gram (+) and (−) bacteria, both aerobic and anaerobic. In vitro data indicate rifaximin is active against species of Staphylococcus, Streptococcus, Enterococcus, and Enterobacteriaceae. Bacterial reduction or an increase in antimicrobial resistance in the colonic flora does not frequently occur and does not have a clinical importance. Rifaximin is currently approved in 17 countries outside the US and was licensed by the Food and Drug Administration (FDA) for the US in May 2004.

In some embodiments, the pharmaceutical compositions and methods described herein may include rifaximin as one or more forms of rifaximin together with other excipients, for example diluting agents such as mannitol, lactose and sorbitol; binding agents such as starches, gelatines, sugars, cellulose derivatives, natural gums and polyvinylpyrrolidone; lubricating agents such as talc, stearates, hydrogenated vegetable oils, polyethylene glycol and colloidal silicon dioxide; disintegrating agents such as starches, celluloses, alginates, gums and reticulated polymers; coloring, flavoring and sweetening agents.

In some embodiments, the pharmaceutical compositions and methods described herein may include rifaximin together with usual excipients, such as white petrolatum, white wax, lanoline and derivatives thereof, stearylic alcohol, red iron oxide, propylene glycol, talc, sodium lauryl sulfate, ethers of fatty polyoxyethylene alcohols, disodium edentate, glycerol palmitostearate, esters of fatty polyoxyethylene acids, sorbitan monostearate, glyceryl monostearate, propylene glycol monostearate, hypromellose, polyethylene glycols, sodium starch glycolate, methylcellulose, hydroxymethyl propylcellulose, sodium carboxymethylcellulose, microcrystalline cellulose, colloidal aluminum and magnesium silicate, titanium dioxide, propylene glycol, colloidal silicon dioxide, or sodium alginate.

In some embodiments of the methods described herein, rifaximin may refer to XIFAXAN. In some embodiments of the methods described herein, rifaximin may refer to the 200 mg dosage form of XIFAXAN. In some embodiments of the methods described herein, rifaximin may refer to one or more tablets of the 200 mg dosage form of XIFAXAN. In some embodiments of the methods described herein, rifaximin may refer to the 550 mg dosage form of XIFAXAN. In some embodiments of the methods described herein, rifaximin may refer to one or more tablets of the 550 mg dosage form of XIFAXAN.

### Pharmaceutical Compositions

In some embodiments, the invention includes pharmaceutical compositions including one or more active agents for treating one or more of the diseases or conditions disclosed herein. In some embodiments, the pharmaceutical composition for treating disease may include a therapeutically effective amount of methylnaltrexone or a salt thereof, a therapeutically effective amount of rifaximin, and a pharmaceutically acceptable carrier. In some embodiments, the pharmaceutical composition for treating disease may include a methylnaltrexone or a salt thereof in an amount or dosage described herein, rifaximin in an amount or dosage described herein, and a pharmaceutically acceptable carrier. In some embodiments, the pharmaceutically acceptable carrier may include one or more of the carriers or excipients described herein.

In some embodiments, the invention includes a pharmaceutical composition comprising methylnaltrexone or a salt thereof and rifaximin, wherein the methylnaltrexone or salt thereof is provided in an amount of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% by weight of the pharmaceutical composition. In some embodiments, the invention includes a pharmaceutical composition comprising methylnaltrexone or a salt thereof and rifaximin, wherein the methylnaltrexone or salt thereof is provided in an amount of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% by weight of the pharmaceutical composition. In some embodiments, the invention includes a pharmaceutical composition comprising methylnaltrexone or a salt thereof and rifaximin, wherein the methylnaltrexone or salt thereof is provided in an amount of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% by weight of the pharmaceutical composition.

In some embodiments, the invention includes a pharmaceutical composition comprising methylnaltrexone or a salt thereof and rifaximin, wherein the rifaximin is provided in an amount of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% by weight of the pharmaceutical composition. In some embodiments, the invention includes a pharmaceutical composition comprising methylnaltrexone or a salt thereof and rifaximin, wherein the rifaximin is provided in an amount of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% by weight of the pharmaceutical composition. In some embodiments, the invention includes a pharmaceutical composition comprising methylnaltrexone or a salt thereof and rifaximin, wherein rifaximin is provided in an amount of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% by weight of the pharmaceutical composition.

Pharmaceutical compositions described herein may be suitable for oral administration and can be presented as discrete dosage forms, such as capsules, sachets, tablets, liquids, or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the pharmaceutical compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added in the pharmaceutical arts as a means of simulating long-term storage in order to determine characteristics such as shelf-life or the stability of pharmaceutical compositions over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, pharmaceutical compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

The active agents can be combined in an intimate admixture with a pharmaceutical carrier according to conventional pharmaceutical compounding techniques. The carrier can take a wide variety of forms depending on the form of preparation desired for administration. In preparing the pharmaceutical compositions for an oral dosage form, any of the usual pharmaceutical media can be employed as carriers, such as, for example, water, glycols (e.g., propylene glycol), oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pregelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the pharmaceutical compositions of the invention to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the active agents disclosed herein. The amount of disintegrant used may vary based upon the type of pharmaceutical composition and mode of administration, and may be readily discernible to those of ordinary skill in the art. About 0.5 to about 15 weight percent of disintegrant, or about 1 to about 5 weight percent of disintegrant, may be used in the pharmaceutical compositions. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl aureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Pharmaceutical compositions for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. For example, a tablet coating may be an Opadry coating (e.g., Opadry II).

Surfactants which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

In an embodiment, the pharmaceutical compositions may include a solubilizer to ensure good solubilization and/or dissolution of the compound of the present invention and to minimize precipitation of the compound of the present invention. A solubilizer may also be added to increase the solubility of the hydrophilic drug and/or other components, such as surfactants, or to maintain the pharmaceutical composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, E-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, caprolactone and isomers thereof, 8-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but are not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropylmethyl cellulose, hydroxypropyl cyclodextrins, ethanol, polyethylene glycol 200-100, glycofurol, transcutol, propylene glycol, and dimethyl isosorbide.

The pharmaceutical compositions described herein can further include one or more pharmaceutically acceptable additives. Such additives include, without limitation, detackifiers, antifoaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

### Methods of Treatment

In an embodiment, a therapeutic agent described herein, such as methylnaltrexone, rifaximin, or a pharmaceutical composition that includes methylnaltrexone and/or rifaximin, may be used in the treatment of disease in a subject in need thereof. In some embodiments, the invention described herein includes a method for treating disease in a subject in need thereof that includes administering a therapeutically effective amount of methylnaltrexone or a salt thereof and a therapeutically effective amount of rifaximin. In some embodiments, the invention described herein includes a method for treating disease in a subject in need thereof that includes administering a pharmaceutical composition comprising a therapeutically effective amount of methylnaltrexone or a salt thereof, a therapeutically effective amount of rifaximin, and a pharmaceutically acceptable carrier. In some embodiments, the disease may be leaky gut syndrome. In some embodiments, the disease may be a disease or condition that results from, is exacerbated by, or is otherwise related to leaky gut syndrome. In some embodiments, the disease may be a condition that may be treated or alleviated by correcting leaky gut syndrome. In some embodiments, the condition that may be treated or alleviated by correcting leaky gut syndrome may be selected from the group consisting of irritable bowel syndrome, inflammatory bowel disease, Crohn's disease, ulcerative colitis, indeterminate colitis, chemotherapy-induced colitis, celiac disease, infectious diarrhea, atopy, allergy, food allergy, asthma, autism, chronic fatigue syndrome, lupus, metabolic syndromes, diabetes, type 1 diabetes, type 2 diabetes, hypertension, hyperlipidemia, neoplasia, cancer, idiopathic inflammatory conditions, rheumatoid arthritis, neurologic disorders, multiple sclerosis, migraines, psoriatic arthritic, autoimmune diseases, rheumatoid arthritis, skin disorders, psoriasis, eczema, acne, metabolic bone disease, osteoporosis in adults, primary growth failure in children, liver disease, fatty liver diseases, nonalcoholic steatohepatitis (NASH), nonalcoholic fatty liver disease (NAFLD), hepatic encephalopathy (HE), anklyosing spondylitis, obesity, graft versus host disease (GVHD), multiple organ dysfunction syndrome, Parkinson's disease, acute pancreatitis, fibromyalgia, mental illness, depression, schizophrenia, burn injury, heart failure, and renal failure. In some embodiments, the disease or syndrome related to, resulting from, or exacerbated by leaky gut may be a liver disease, such as fatty liver disease. In some embodiments, the condition that may be treated or alleviated by correcting leaky gut syndrome may be cirrhosis. In some embodiments, the condition that may be treated or alleviated by correcting leaky gut syndrome may be obesity, a fatty liver disease, celiac disease, Crohn's disease, and irritable bowel syndrome. In some embodiments, a fatty liver disease may be NASH or NAFLD. In some embodiments, the condition that may be treated or alleviated by correcting leaky gut syndrome may be HE.

In some embodiments, the invention may include a method of treating increased gut permeability in a subject in need thereof, by administering a therapeutically effective amount of at least one therapeutic agent selected from the group consisting of methylnaltrexone, or a salt thereof and rifaximin. In some embodiments, the invention may include a method of reducing gut permeability in a subject in need thereof, by administering a therapeutically effective amount of a therapeutic agent selected from the group consisting of methylnaltrexone, or a salt thereof, rifaximin, and a combination thereof. In some embodiments, the invention may include a method of treating a disease or condition associated with increased gut permeability in a subject in need thereof, the method comprising administering an amount of a therapeutic agent selected from the group consisting of methylnaltrexone, or a salt thereof, rifaximin, and a combination thereof. In some embodiments, the invention may include a method of treating NASH or a symptom thereof in a subject in need thereof, by administering a therapeutically effective amount of a therapeutic agent selected from the group consisting of methylnaltrexone, or a salt thereof, rifaximin, and a combination thereof. In some embodiments, the invention may include a method of treating NAFLD or a symptom thereof in a subject in need thereof, by administering a therapeutically effective amount of a therapeutic agent selected from the group consisting of methylnaltrexone, or a salt thereof, rifaximin, and a combination thereof. In some embodiments, the invention may include a method of treating HE or a symptom thereof in a subject in need thereof, by administering (i) a therapeutically effective amount of methylnaltrexone, or a salt thereof; or (ii) a therapeutically effective amount of methylnaltrexone, or a salt thereof, and rifaximin.

Increased intestinal permeability, or the abnormal and excessive opening of intercellular tight junctions, may allow for the passage of foreign objects, such as a single molecules or digested food molecules, and microbes, microbial products, and foreign antigens into the mucosa and bloodstream, which can result in subsequent development of inflammatory and/or immune reactions. The foregoing condition that results in increased intestinal permeability and the excessive opening of intercellular tight junctions is referred to herein as “leaky gut syndrome” or simply “leaky gut.” Leaky gut syndrome is implicated in or may result in the development of additional gastrointestinal pathologies including, for example, obesity, fatty liver diseases (e.g., NASH and NAFLD), celiac disease, Crohn's disease, and irritable bowel syndrome (IBS).

Note that in leaky gut syndrome, a break or opening in the tight junctions can be very small (e.g., to only allow larger molecules such as simple sugars to enter the body from the lumen of the GI tract), or can be larger (e.g., to allow bacteria and cells to enter the body from the lumen of the GI tract). Ideally, leaky gut syndrome will be detected while the break is still very small, and before the break is large enough to allow entry of a large foreign body, such as a virus or bacteria.

Subjects with leaky gut syndrome (e.g., subjects having a disease or condition associated with increased gut permeability) may already have or may develop symptoms of irritable bowel syndrome, inflammatory bowel disease (e.g., Crohn's disease, ulcerative colitis, indeterminate colitis and chemotherapy-induced colitis), celiac disease, infectious diarrhea, atopy, allergy (e.g., food allergy), asthma, autism, chronic fatigue syndrome, lupus, metabolic syndromes (including, but not limited to, diabetes, such as type 1 diabetes and/or types 2 diabetes, hypertension, and hyperlipidemia), neoplasia or cancer, idiopathic inflammatory conditions (e.g. rheumatoid arthritis), neurologic disorders (e.g. multiple sclerosis), migraines, psoriatic arthritic, autoimmune diseases such as rheumatoid arthritis and psoriasis, skin disorders such as eczema and acne, metabolic bone disease (including but not limited to osteoporosis in adults and primary growth failure in children), liver disease such as fatty liver diseases (including NASH and NAFLD), hepatic encephalopathy (HE), anklyosing spondylitis, obesity, graft versus host disease (GVHD), multiple organ dysfunction syndrome, Parkinson's disease, acute pancreatitis, fibromyalgia, mental illness such as depression and schizophrenia, burn injury, heart failure, renal failure and other disease indications that systemic or related to the GI tract. Leaky gut can also arise in subjects, such as elderly subjects or children, compromising the ability of the subject to absorb nutrients from consumed food. In some embodiments, the disease or condition associated with increased gut permeability may be a liver disease. In some embodiments, the disease or condition associated with increased gut permeability may be a fatty liver disease. In some embodiments, the disease or condition associated with increased gut permeability may be NASH. In some embodiments, the disease or condition associated with increased gut permeability may be NAFLD. In some embodiments, the disease or condition associated with increased gut permeability may be hepatic encephalopathy (HE).

In some embodiments, leaky gut results when tight junctions between individual cells at the GI barrier become loosened, allowing particles and potentially microbes to pass through the junction from the lumen and into the body. The loosening of the tight junctions may be due, for example, crenation or shrinkage of the cells, thereby widening the junction between the crenated cell and its adjacent cell.

In some embodiments, the leaky gut results from other types of damage to the cells at the GI barrier. For example, the cells at the barrier may become inflamed or may start expressing proteins involved in programmed cell death (e.g., apoptosis, pyroptosis and necroptosis).

### Fatty Liver Diseases

Fatty liver disease may include non-alcoholic fatty liver disease (NAFLD) and non-alcoholic steatohepatitis (NASH). NAFLD includes a wide spectrum of liver abnormalities which range from simple steatosis to (NASH). NASH is a severe form of NAFLD, where excess fat accumulation in the liver results in chronic inflammation and damage. Nonalcoholic fatty liver disease is a component of metabolic syndrome and a spectrum of liver disorders ranging from simple steatosis to nonalcoholic steatohepatitis (NASH). Simple liver steatosis is defined as a benign form of NAFLD with minimal risk of progression, in contrast to NASH, which tends to progress to cirrhosis in up to 20% of subjects and can subsequently lead to liver failure or hepatocellular carcinoma. NASH affects approximately 3-5% of the population in America, especially in those identified as obese. NASH is characterized by such abnormalities as advanced lipotoxic metabolites, pro-inflammatory substrate, fibrosis (e.g., in which collagen deposition is manifested in a particular perivenular and/or pericellular pattern), and increased hepatic lipid deposition. If left untreated, NASH can lead to cirrhosis, liver failure, and hepatocellular carcinoma.

Although subjects diagnosed with alcoholic steatohepatitis demonstrate similar symptoms and liver damage, NASH develops in individuals who do not consume alcohol, and the underlying causes of NASH are unknown. Hepatic steatosis occurs when the amount of imported and synthesized lipids exceeds the export or catabolism in hepatocytes. An excess intake of fat or carbohydrate is the main cause of hepatic steatosis. NAFLD subjects exhibit signs of liver inflammation and increased hepatic lipid accumulation. In addition, the development of NAFLD in obese individuals is closely associated with insulin resistance and other metabolic disorders and thus might be of clinical relevance. Therefore, possible causative factors include insulin resistance, cytokine imbalance (specifically, an increase in the tumor necrosis factor-alpha (TNF-a)/adiponectin ratio), and oxidative stress resulting from mitochondrial abnormalities.

Although the majority of subjects can be effectively diagnosed with NAFLD using noninvasive tests, liver biopsy is needed for the accurate assessment of the graduation of steatosis, necroinflammatory changes, and fibrosis and allows NASH and steatosis to be distinguished (Sanches et al., Nonalcoholic Steatohepatitis: A Search for Factual Animal Models BioMed Research International Volume 2015). At least two grading systems for NASH have been developed, which take into consideration the severity of hepatic steatosis, portal and lobular inflammation, and collagen deposition.

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

All documents, or portions of documents, cited in this application, including but not limited to patents, patent applications, articles, books, and treatises, are hereby expressly incorporated by reference in their entirety for any purpose.

### EXAMPLES

The following Examples are merely illustrative and are not intended to limit the scope or content of the disclosure in any way.

### Example 1: MNTX and Rifaximin Treatments Dramatically Improved Western Diet-Induced Gut Permeability in the Diamond™ Mouse Model

Method:

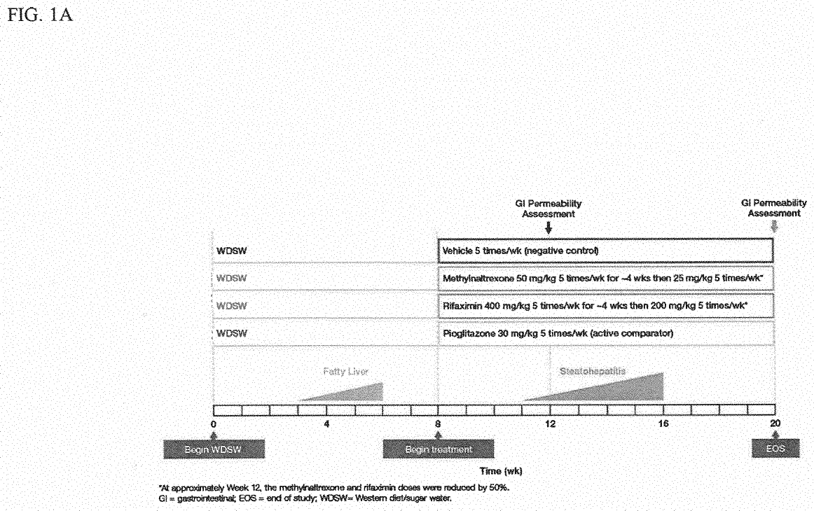

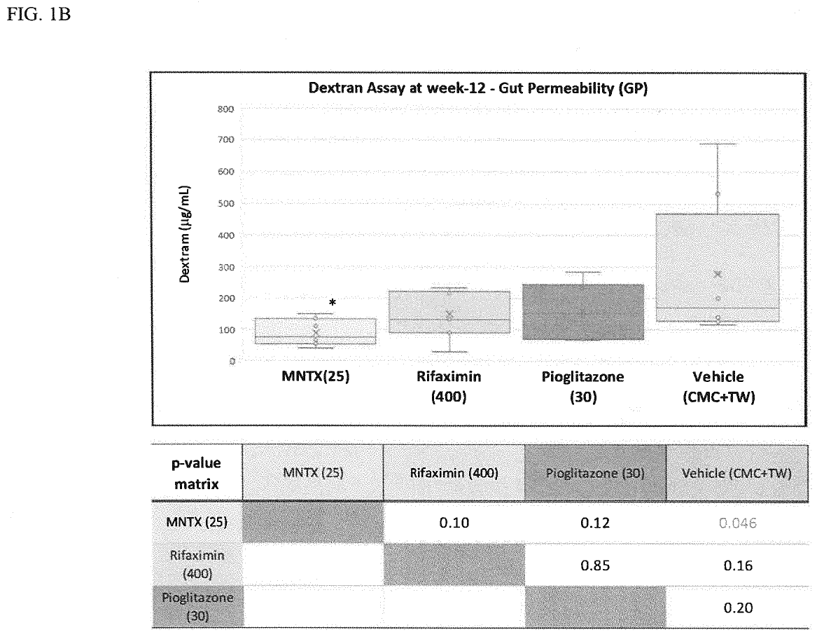

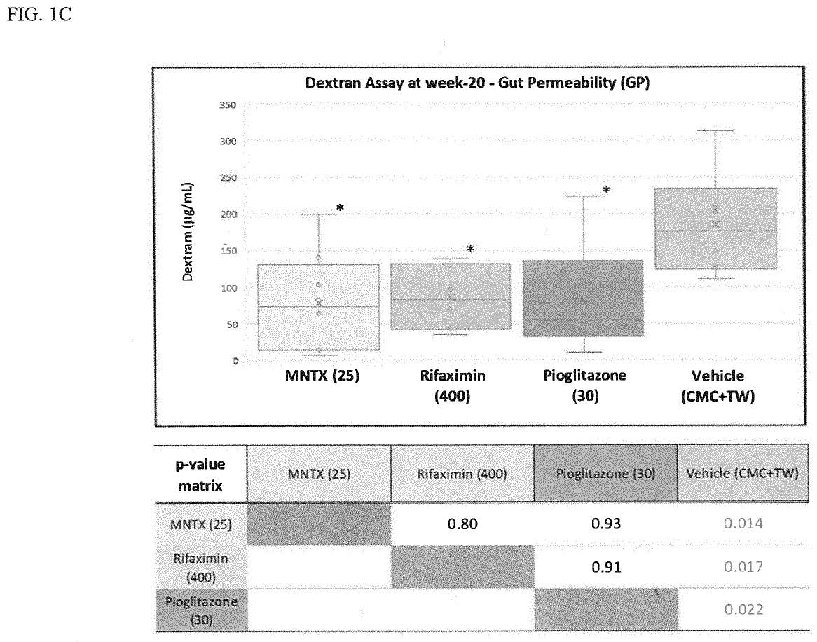

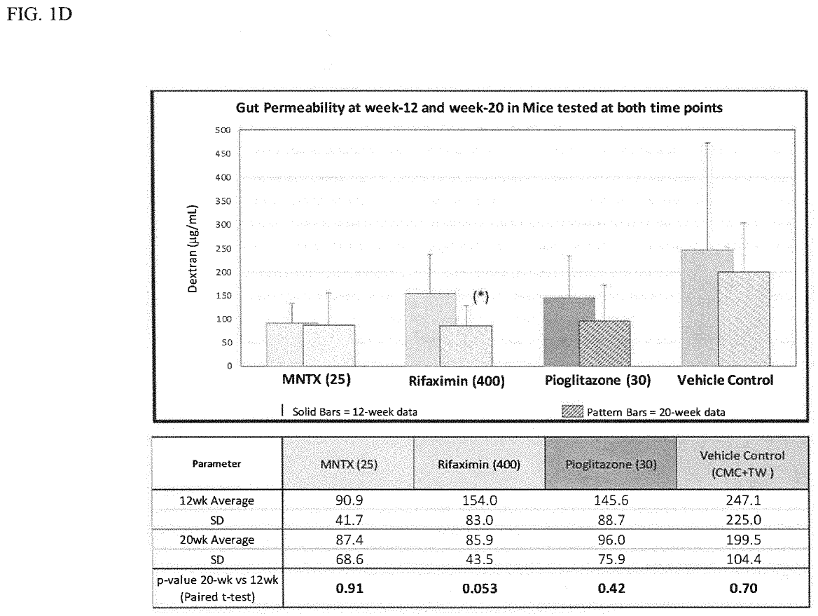

Four groups of 8 DIAMOND™ mice were place on a western diet with sugar water (WDSW; Teklad 42% high fat diet, HFD, and 4% sugar water) for 8 weeks then continued on WDSW with oral gavage of either aqueous vehicle, methylnaltrexone (MNTX), Rifaximin, or pioglitazone 5×/week for 12 weeks (FIG. 1A). MNTX was administered at 50 mg/kg and Rifaximin at 400 mg/kg for 4 weeks, then MNTX dose was reduced to 25 mg/kg and Rifaximin to 200 mg/kg for the remaining 8 weeks of the treatment period. Pioglitazone was dosed at 30 mg/kg for the entire 12 weeks. Gut permeability (GP) was assessed twice at 12 weeks and 20 weeks by measuring dextran absorption into the bloodstream using a competitive enzyme-linked immunosorbent assay (ELISA) with 20 μL of serum collected by tail nick. Mice were gavaged with 4 kDa dextran at 600 mg/kg; the more permeable the gut, the more dextran is detected in serum. Results between dose groups were compared using Student's 2-tailed t-test, and pair-wise analysis was performed on serial measurements from week-12 to week-20 to assess drug impact on progression of GP over time. Serum and liver tissues were collected at necropsy, formalin-fixed, paraffin-embedded (FFPE) slides stained with H&E and Sirius Red, and liver pathology was assessed.

The data for this experiment was analyzed twice, as described below.

Results Analysis 1:

Average serum dextran concentrations of vehicle control (VC) WDSW mice at 12 weeks (corresponding to NASH F0) were high, averaging 247 μg/ml, confirming that as expected GP was increased due to diet. MNTX treatment reduced GP quickly, after only 4 weeks of treatment; at the 12 week timepoint, the serum dextran concentration was 90.9 μg/ml, a 2.7-fold reduction compared to the VC group (P≤0.046) (FIG. 1B). Both MNTX and Rifaximin also successfully reduced GP at the 20 week timepoint (12 weeks of treatment); the average serum dextran concentrations were 2-fold lower than the VC group for MNTX and Rifaximin (P≤0.014 and P≤0.017, respectively) (FIG. 1C). Treatment with pioglitazone, the insulin sensitizer used as an active comparator, also improved GP compared to VC at 20 weeks (FIG. 1C).

By pair-wise analysis, Rifaximin treatment continued improving the leaky gut induced by diet over time; at 20 weeks the serum dextran in the Rifaximin-treated group averaged 85.9 μg/ml, compared to the 12-week average of 154.0 μg/ml (FIG. 1D). Thus, Rifaximin improved leaky gut by 45% in the 8 weeks between the first and second measurement, and this improvement was statistically significant (P≤0.053) (FIG. 1D).

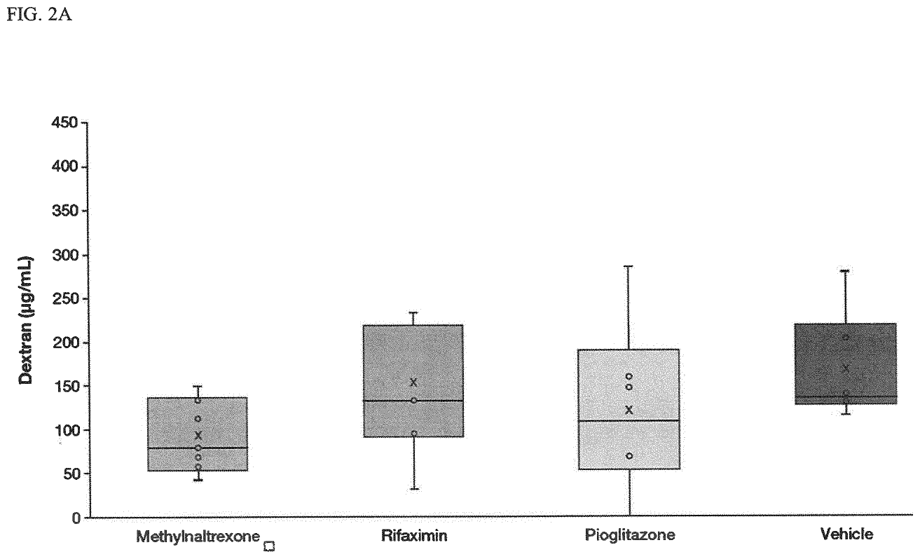

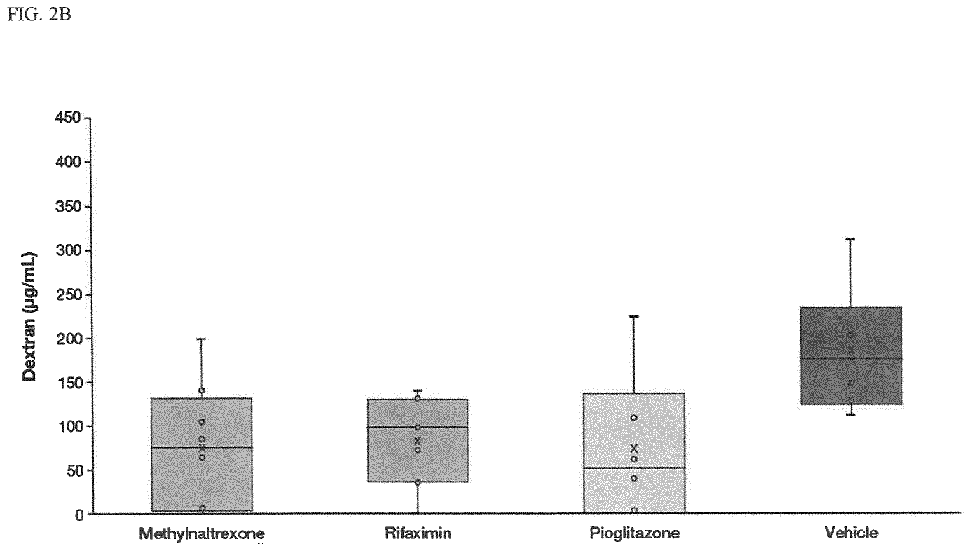

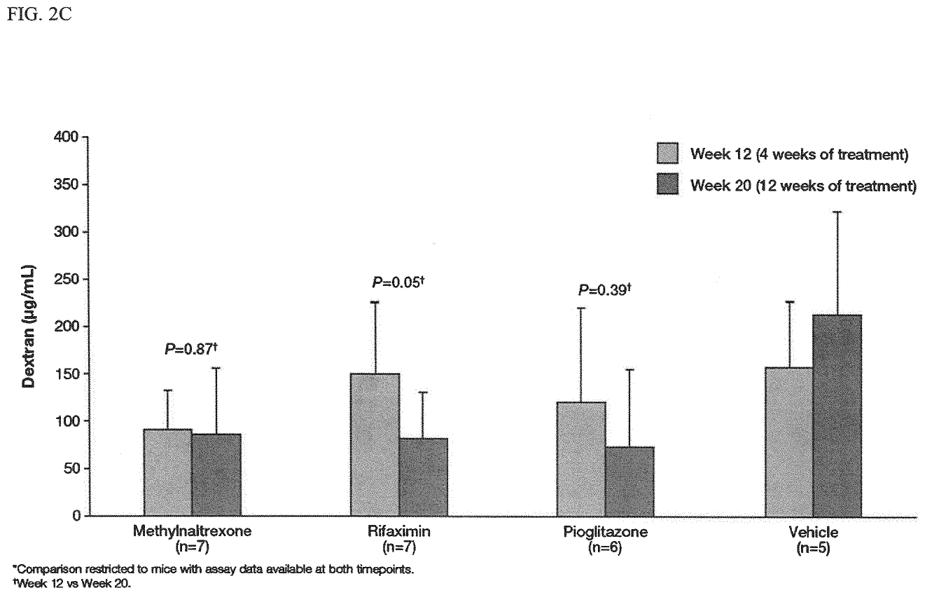

Results Analysis 2: After 4 weeks of treatment (Week 12; corresponding to NASH F0), mean serum dextran concentrations were 166 μg/mL for vehicle, 91 μg/mL for methylnaltrexone, 151 μg/mL for rifaximin, and 121 μg/mL for pioglitazone, the active comparator (FIG. 2A) A significant reduction in serum dextran concentration was observed with methylnaltrexone. Treatment with methylnaltrexone resulted in a 1.8-fold reduction in mean serum dextran concentrations compared with vehicle (P=0.04). There were no significant differences for rifaximin or pioglitazone (active comparator) relative to vehicle.

After 12 weeks of treatment (Week 20), a significant reduction in serum dextran concentration was observed with methylnaltrexone, rifaximin, and pioglitazone (corresponding to NASH F1; FIG. 2B) Mean serum dextran concentrations were 215.6 μg/mL for vehicle, 74.7 μg/mL for methylnaltrexone, 81.2 μg/mL for rifaximin, and 71.8 μg/mL for pioglitazone. Compared with vehicle, mean serum concentrations were reduced by 2.9 fold with methylnaltrexone (P=0.01), 2.7-fold with rifaximin (P=0.01), and 3.0-fold with pioglitazone (P=0.02). Pair-wise analysis showed a trend over time with rifaximin treatment in decreasing GI permeability induced by WDSW, with a 46.2% improvement in “leaky gut” during the 8 weeks between assessments (P=0.05; FIG. 2C) In contrast, vehicle tended to increase gut permeability between Weeks 12 and 20, but the difference was not significant (P=0.4).

Conclusion:

MNTX and Rifaximin treatments dramatically improved Western Diet-induced GP in the DIAMOND™ model, suggesting that the compounds may have broad utility in treating inflammatory diseases exacerbated by “leaky gut”. These results support the biological rationale for further testing these compounds for efficacy in NASH, particularly in combination with drugs that hit other mechanistic drivers such as insulin sensitivity and inflammation. Further dose optimization studies are needed (amount, route of administration) to determine if these compounds could be effective NASH monotherapies.

### Example 2: Evaluation of the Effects of Methylnaltrexone, Rifaximin, and Rifaximin in Combination with Methylnaltrexone Bromide (MNTX) on Inflammation, Nash, and Progression of Fibrosis and Gut Permeability, in the Diamond™ (Diet Induced Animal Model of Non-Alcoholic Fatty Liver Disease) Mice

### SUMMARY

Method:

DIAMOND™ male mice were put on special diet consisting of western diet (Teklad 42% fat, WD) and sweetened water (D-Fructose and D-Glucose, SW) beginning at t=0. Mice were weighed weekly.

At 8 weeks on diet (t=8), mice were randomized into experimental groups based on the most recent body weight. Test compounds were administered by oral gavage as follows:
Methylnaltrexone bromide (MNTX)—50 mg/kg (weeks 8-12) and 25 mg/kg (weeks 12-20)
Rifaximin—400 mg/kg (weeks 8-12) and 200 mg/kg (weeks 12-20)
Rifaximin—400 mg/kg with MNTX—50 mg/kg (weeks 8-12), and rifaximin—200 mg/kg with MNTX—25 mg/kg (weeks 12-20)
Pioglitazone—30 mg/kg
Vehicle—0.1% CMC with 0.01% Tween 80
   At approximately Week 12, methylnaltrexone dose was reduced to 25 mg/kg body weight and rifaximin dose was reduced to 200 mg/kg body weight.
   Gut integrity/permeability was measured with the leaky gut assay on week 12 (one month on test article treatment) and on week 20 immediately prior to sacrifice. At each assay time point, between 0.2 ml and 0.4 ml of 4 kDa dextran solution (2 g/10 ml PBS solution, 0.2 g/mL) was orally gavaged to mice. After 4 hours, blood was collected from the tail vein and processed to serum. Dextran concentration in samples was measured by ELISA.

Results:

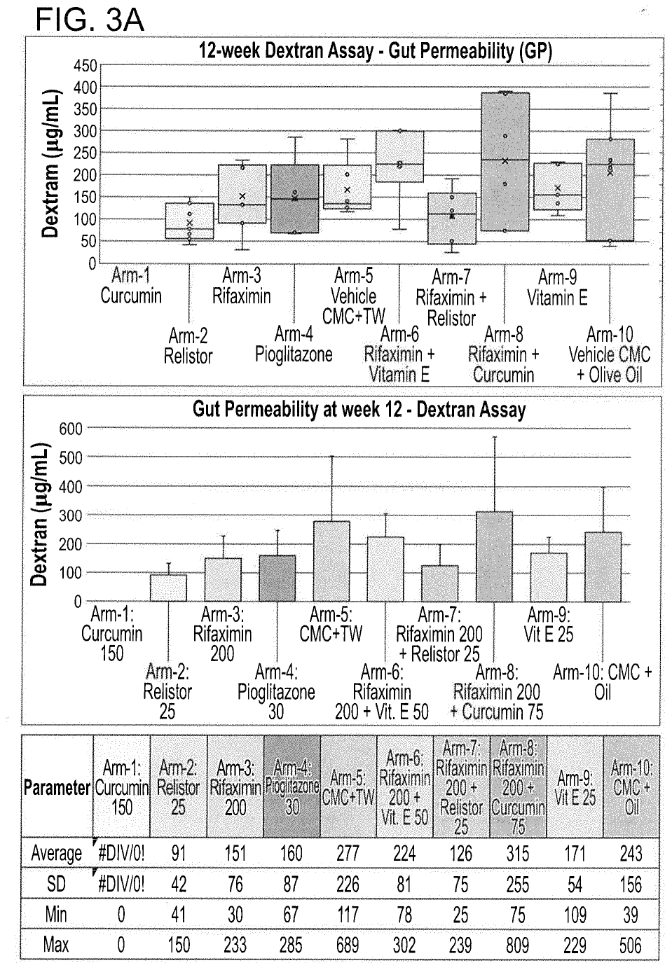

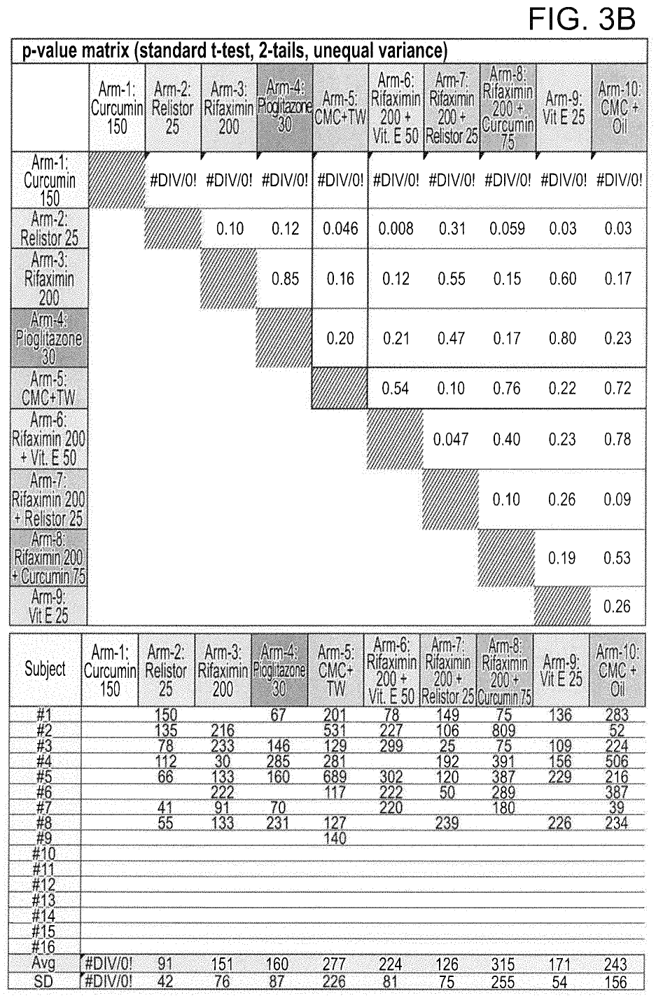

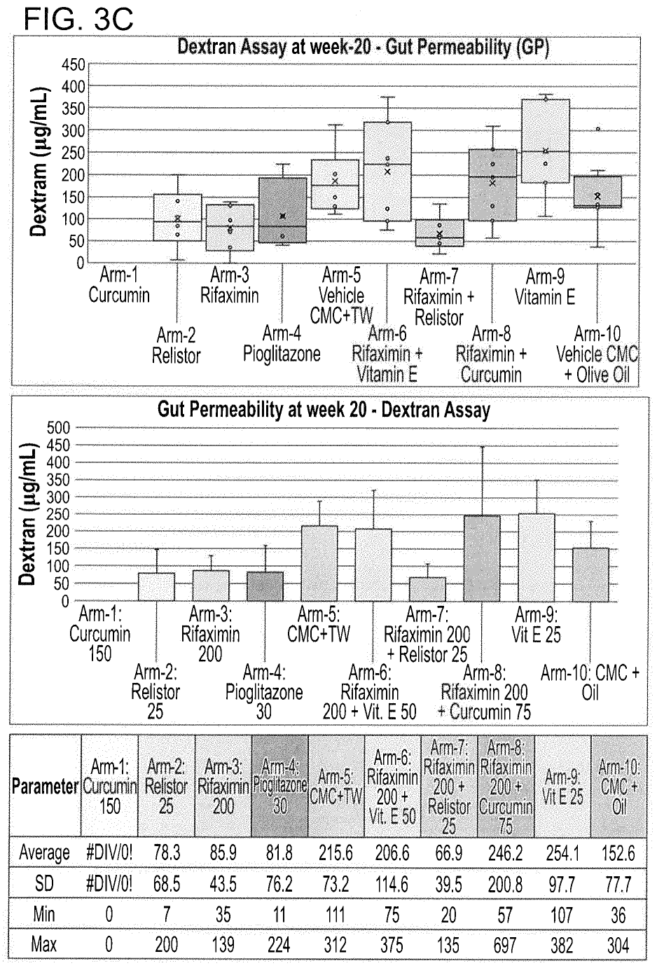

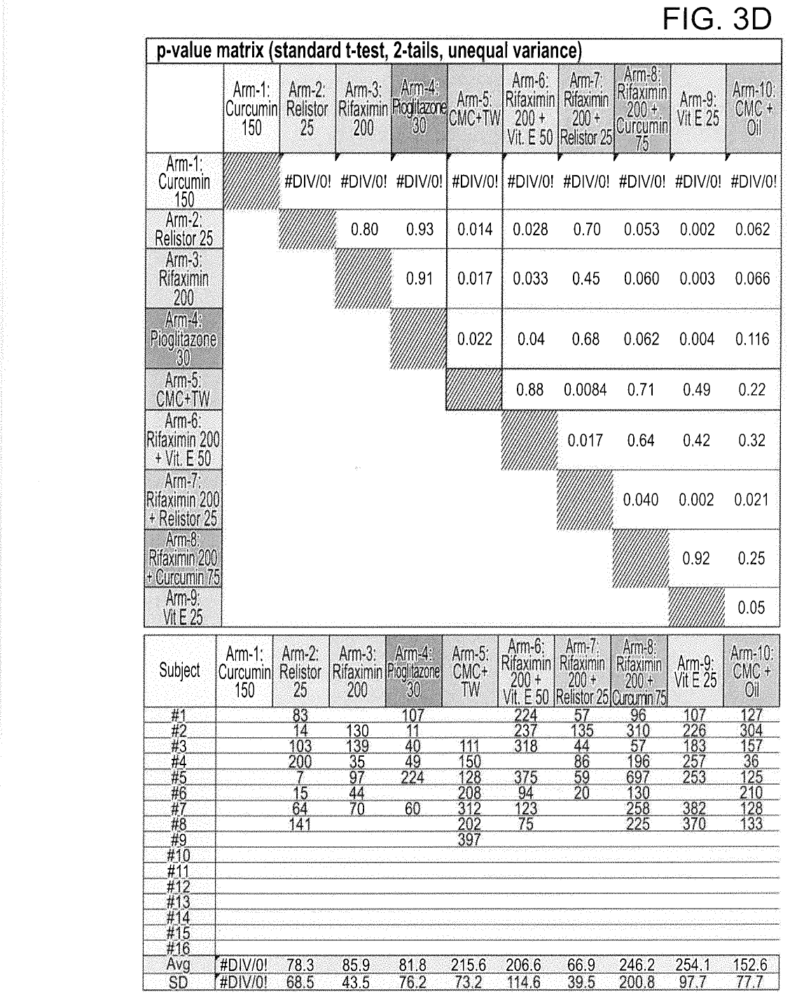

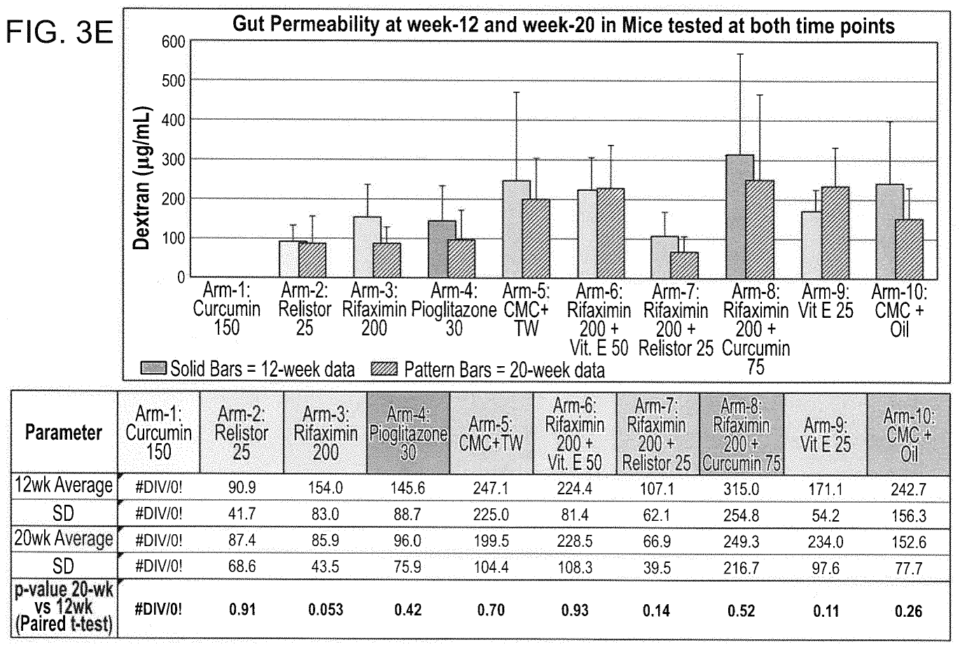

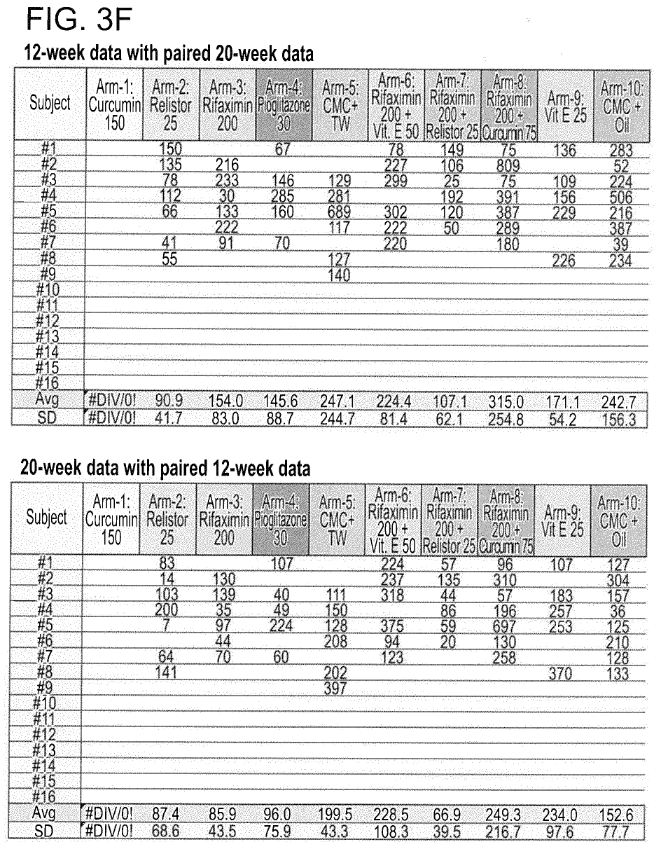

At week-12, very few differences in GP were noted between the study arms (see FIGS. 3A-3B). The MNTX arm (Arm-2) displayed a significantly lower GP when compared to the vehicle control (Arm-5). At week-20, several significant changes were noted between the different study arms (see FIGS. 3C-3D). The significantly lower GP noted at week-12 for MNTX (Arm-2) persists at week-20 suggesting a sustained effect of MNTX on GP. Whereas no changes were noted at week-12, by week-20 there was a significantly lower GP in that arm when compared to the vehicle control (Arm-5). This suggests that the effect of Rifaximin (Arm-3) on GP develops in time; a finding consistent with the fact that Rifaximin is an antibiotic whose effectiveness develop over time rather than instantly as might be the case for MNTX. The combination of MNTX and Rifaximin (Arm-7) tends to be associated with the lowest GP although the actual GP value for Arm-7 is not statistically lower than in the MNTX-only or Rifaximin-only arms (Arm-2 and Arm-3, respectively). This suggests that the combination does not interfere with the effectiveness of either or both drugs. Week-20 vs. Week-12 (see FIGS. 3E-3F).

Conclusion:

Overall it was observed that MNTX's effect is maintained but does not increase (or lose) over time. The trend toward lower GP for Rifaximin-alone (Arm-3) is borderline significant (p=0.051). Trends toward higher GP were noted too but none are close to statistical significance (see FIGS. 3E-3F).

### Detailed Methodology and Results

Ten different Arms were used with 8 to 10 mice per Arm. Two vehicle control Arms were used to account for the differential solubility of the tested compounds and their combinations.

Gut Permeability (GP).

MNTX was the only one to trigger a lower GP only 4 weeks into the treatment period (thus at 12 weeks).

Within the 8-week treatment period (at week 20), all arms with MNTX, Rifaximin and both had statistically significant lower GP.

Pioglitazone joined that trend with GP significantly lower than in the control group (Arm-5) but not any lower than in the MNTX, Rifaximin and MNTX+Rifaximin arms.

Body Weight and Liver Weight.

No statistically significant difference in body weight between any arm vs. the control arms. Liver weights were lower in all combination therapy arms as well as in the MNTX alone and the Vitamin-E alone arms. However, these observations need to be put in perspective since in the second control Arm (Arm-10, CMC+Olive Oil) the liver weight was statistically significantly decreased.

Biochemical Markers.

There were not many significant changes triggered by the different treatments but for those changes, there did not seem to be a consistent trend or theme; for example, Glucose was slightly decreased in Arm-6 (Rifaximin+Vit-E) only, Ketones slightly decreased in Arm-3 (Rifaximin) and Arm-4 (Pioglitazone).

Liver Enzymes.

ALT and AST were significantly increased with Vitamin E, there was a trend toward lower levels in Arm-6 (Rifaximin and Vitamin-E) and Arm-7 (Rifaximin and MNTX) but not significantly. ALP, TBIL and GGT were unaffected all groups. Albumin was decreased only in Arm-9 (Vitamin-E). BUN saw multiple significant changes, but these also included a significant increase in the second control Arm (Arm-10, CMC+Olive Oil).

Histology Findings.

The most consistent result was the effect of Vitamin-E alone (Arm-9) on Lobular Inflammation (increased, somewhat counter-intuitive), NAS and SAF Scores (both increased). Steatosis (%) was increased in Arm-3 (Rifaximin alone) but not in any Arms with a Rifaximin combination. Pioglitazone was the only treatment arm that had no NASH specimen.

Disease Category.

Only the pioglitazone Arm had no prevalence of NASH. All other groups had equal or higher prevalence of NASH (from 14 to 50%) than either controls arms (11% in Arm-5 and 0% in Arm-10). Treatment increasing NASH prevalence were MNTX (Arm-2, 38% prevalence of NASH), Vitamin E (Arm-9, 38% prevalence of NASH), Rifaximin+Vitamin E (Arm-6, 25% prevalence of NASH), and the MNTX-Rifaximin combo (Arm-7, 50% prevalence of NASH).

In conclusion, a certain level of gut permeability was characterized in the DIAMOND mouse model. Treatment with different test article and their combination showed that GP can be lowered. However, GP reduction, in this mouse model under this specific protocol, prevented the development/progression of liver disease; in some instances, the prevalence of an advanced liver disease stage (NASH) seemed to be increased by the use those test articles.

If an increase in gut permeability is linked to the development or progression of NASH (in humans) by allowing gut microbiome end products to reach and harm the liver, in the study here, this microbiome-liver axis was not at play. It is possible that the microbiome in the DIAMOND mice in this study with this diet was not eliciting the generation of harmful compounds that would trigger the development of NASH.

It is possible that should a (standardized) gut microbiome be identified as harmful to the liver, a fecal transplant into the DIAMOND mice might be able to answer whether this gut microbiome contributes to disease progression and whether lowering gut permeability would prevent some of this progression. Here, it seems that most of the features of liver disease and NASH were brought forth by the diet itself (WDSW) independently of the gut microbiome-liver axis.

### Test Compounds

Dose formulations were prepared at the following concentrations in a suitable vehicle:

1) Compound: Vitamin E (a-tocopherol)—20 mg/ml in 0.01% carboxymethylcellulose (CMC) with 0.01% Tween 80
2) Compound: Methylnaltrexone bromide—10 mg/ml 0.01% carboxymethylcellulose (CMC) with 0.01% Tween 80
3) Compound: Curcumin, dose—30 mg/ml in olive oil
4) Compound: Rifaximin, dose—80 mg/ml in olive oil
5) Compound: Pioglitazone, dose—20 mg/ml in 0.01% CMC with 0.01% tween 80
6) Vehicle 1—0.01% CMC with 0.01% Tween 80

### 7) Vehicle 2—Olive Oil

### Test System

### 1) Animal Model

a. DIAMOND Mice
b. Male C57BL/6J(B6)-129s1/SvImJ(S129)
c. Supplier: Sanyal Biotechnology LLC
d. 8-9 weeks of age at study initiation

### 2) Food

a. Western Diet (WD)—Treatment and Vehicle Control Mice
   i. ENVIGO
   ii. Teklad 42% calories from fat
   iii. TD. 88137

### 3) Water

a. Sugar Water (SW)—Treatment and Vehicle Control Mice
   i. D-fructose (Alfa Aesar #A17718)
   ii. D-glucose—(Fisher Scientific #D19-212)
   iii. Recipe=231 g Fructose+189 g Glucose+750 ml RO water, then further diluted into 9 L RO water

### 4) Animal Identification

a. Ear tag ID #0001-191; numbers are not sequential and will be assigned based on mouse availability.
b. Cage cards list date of birth, date of weaning, sire/dam, date on western diet sugar water, health status, ear tag number, cage mates, and drug dose group.

### 5) Animal Housing

a. Techniplast high density positive pressure ventilated racks (n=5 animals/cage unless aggressive)
b. Animal room with 12-hour light/dark cycle

### Study Summary

Eighty-two (82) male DIAMOND™ male mice placed on the WDSW diet ad libitum and allowed to develop steatohepatitis and NASH after consuming WDSW diet for 8 weeks.

While on WDSW diet throughout the study, animals were dosed with vehicle or test compounds beginning on week-8 and continuing through week-20.

### Arm-1:

8 mice gavaged with Curcumin at a dose of 150 mg/kg. It is noted that Arm-1 was terminated early due to excess mortality and was not assayed for gut permeability.

### Arm-2:

8 mice gavaged with Methylnaltrexone bromide (MNTX) at a dose of 50 mg/kg (weeks 8-12) and 25 mg/kg (weeks 12-20).

### Arm-3:

8 mice gavaged with Rifaximin at a dose of 400 mg/kg (weeks 8-12) and 200 mg/kg (weeks 12-20).

### Arm-4:

8 mice gavaged with Pioglitazone at a dose of 30 mg/kg.

### Arm-5:

10 mice gavaged with Vehicle-1 (0.01% carboxylmethylcellulose (CMC) with 0.01% Tween-80)

### Arm-6:

8 mice gavaged with Rifaximin at a dose of 400 mg/kg and Vitamin E 100 mg/kg (weeks 8-12), then Rifaximin at a dose of 200 mg/kg and Vitamin E 50 mg/kg (weeks 12-20). Vitamin E was dosed 6 hours after rifaximin.

### Arm-7:

8 mice gavaged with Rifaximin at a dose of 400 mg/kg and MNTX at 50 mg/kg (weeks 8-12), then Rifaximin at a dose of 200 mg/kg and MNTX at 25 mg/kg (weeks 12-20).

### Arm-8:

8 mice gavaged with Rifaximin at a dose of 400 mg/kg and Curcumin at 150 mg/kg (weeks 8-12), then Rifaximin at a dose of 200 mg/kg and Curcumin at 75 mg/kg (weeks 12-20)1. Curcumin was dosed 6 hours after rifaximin.

### Arm-9:

8 mice gavaged with Vitamin E (a-tocopherol) at a dose of 100 mg/kg (weeks 8-12) and 50 mg/kg (weeks 12-20).

### Arm-10:

8 mice gavaged consecutively with the two vehicles, 0.1% CMC with 0.01% Tween 80, and olive oil. Olive oil dosed approximately 6 hours after 0.1% CMC with 0.01% Tween 80.

Gut integrity/permeability was measured with the leaky gut assay at two time points (12-week and 20-week) by competitive Dextran ELISA, 4 weeks after initial drug dosing with test article or vehicles, and immediately prior to sacrifice. Fresh fecal pellets were collected prior to each assay and placed into RNAlater and frozen (for possible future gut microbiome analysis).

At week-20, all mice euthanized and necropsied, the liver and serum collected, and the tissues split between flash frozen (stored frozen at −80° C. in RNAlater) and fixed-formalin samples. Gut contents from cecum and colon was collected for each mouse at necropsy and frozen in RNAlater.

### Experimental Design Details

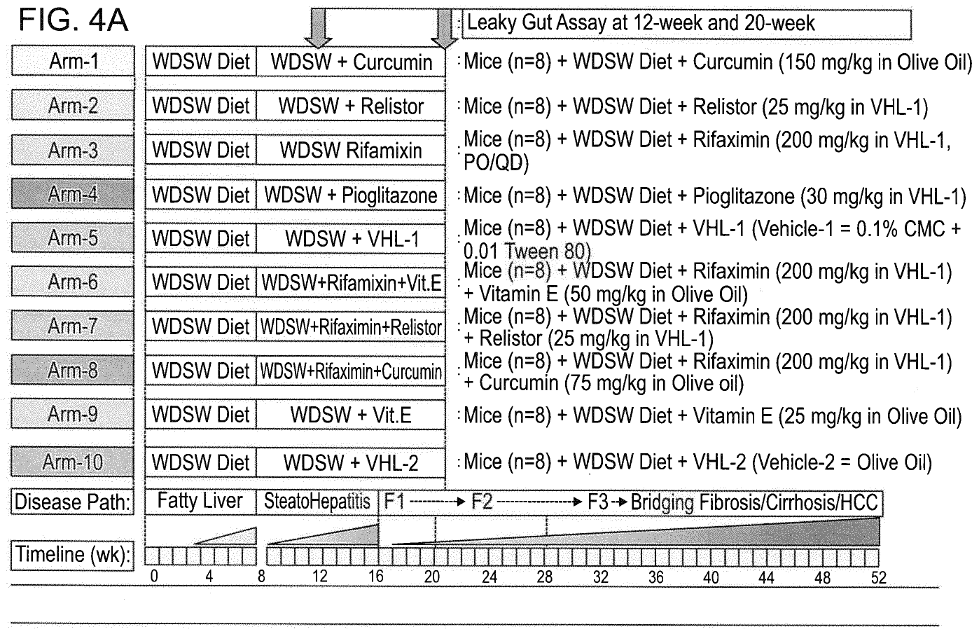

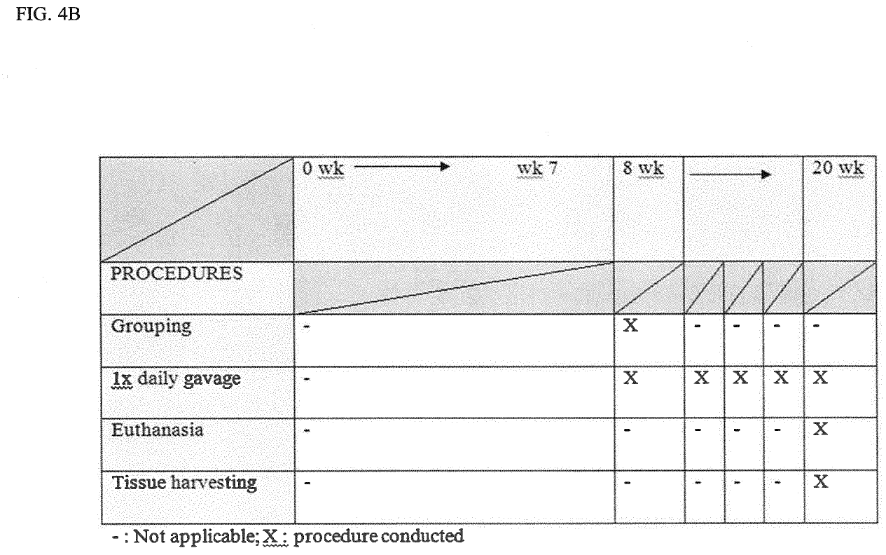

The experimental design details are shown in FIG. 4A. Eighty-two (82) DIAMOND™ male mice were put on the special diet (western diet Teklad 42% fat, WD) and sweetened water (D-Fructose and D-Glucose, SW) at time point 0 (t=0). Mice were weighed weekly starting at t=0. At t=0, the health status and body weight were monitored and recorded in a study notebook. At 8 weeks on diet (t=8) and immediately prior to the first dose, mice were randomized into ten (10) experimental groups; randomization was based on the most recent body weight.

Five (5) mice were kept in each cage unless they became aggressive. Mice on WDSW had ad lib access to food via overhead hoppers and to water via bottles throughout the study. Mice were kept on a 12-hour light/dark cycle and held in Techniplast high density positive pressure ventilated racks.

### Arm-1:

8 mice dosed from week 8 (t=8) until week 20 (t=20) on the Western Diet Sugar Water with once (1×) daily oral gavage with curcumin 150 mg/kg. It is noted that Arm-1 was terminated early due to excess mortality and was not assayed for gut permeability or other endpoints.

### Arm-2:

8 mice dosed from week 8 (t=8) until week 20 (t=20) on the Western Diet Sugar Water with once (1×) daily oral gavage with MNTX 50 mg/kg (weeks 8-12) and 25 mg/kg (weeks 12-20.

### Arm-3:

8 mice dosed from week 8 (t=8) until week 20 (t=20) on the Western Diet Sugar Water with once (1×) daily oral gavage with rifaximin 400 mg/kg (weeks 8-12) and 200 mg/kg (weeks 12-20).

### Arm-4:

8 mice dosed from week 8 (t=12) until week 20 (t=20) on the Western Diet Sugar Water with once (1×) daily oral gavage with pioglitazone (30 mg/kg).

### Arm-5:

10 mice dosed from week 8 (t=8) until week 20 (t=20) on the Western Diet Sugar Water with once (1×) daily oral gavage with 0.1% CMC with 0.01% Tween 80.

### Arm-6:

8 mice dosed from week 8 (t=12) until week 20 (t=20) on the Western Diet Sugar Water with once (1×) daily oral gavage with Rifaximin 400 mg/kg and Vitamin E 100 mg/kg (weeks 8-12), then Rifaximin at a dose of 200 mg/kg and Vitamin E 50 mg/kg (weeks 12-20). Vitamin E was dosed 6 hours after rifaximin.

### Arm-7:

8 mice dosed from week 8 (t=12) until week 20 (t=20) on the Western Diet Sugar Water with once (1×) daily oral gavage with rifaximin 400 mg/kg and MNTX 50 mg/kg (weeks 8-12), then Rifaximin at a dose of 200 mg/kg and MNTX (MNTX) at 25 mg/kg (weeks 12-20).

### Arm-8:

8 mice dosed from week 8 (t=12) until week 20 (t=20) on the Western Diet Sugar Water with once (1×) daily oral gavage with rifaximin 400 mg/kg and curcumin 150 mg/kg (weeks 8-12), then Rifaximin at a dose of 200 mg/kg and Curcumin at 75 mg/kg (weeks 12-20). Curcumin was dosed 6 hours after rifaximin.

### Arm-9:

8 mice dosed from week 8 (t=12) until week 20 (t=20) on the Western Diet Sugar Water with once (1×) daily oral gavage with vitamin E 100 mg/kg (weeks 8-12) and 50 mg/kg (weeks 12-20).

### Arm-10:

8 mice dosed from week 8 (t=8) until week 20 (t=20) on the Western Diet Sugar Water with once (1×) daily oral gavage with 0.1% CMC with 0.01% Tween 80. Mice were gavaged with olive oil approximately 6 hours later.

Following completion of the dosing period on t=20, the mice were placed into a clean cage and fasted for six (6) hours prior to euthanasia. Animals were euthanized by exposing them to carbon dioxide as an anesthetic followed by cardiac puncture for exsanguination. Cervical dislocation was performed as a secondary means of euthanasia. A volume of 3-5 μl of whole blood were collected from a tail nick premortem and used for glucose and ketone testing. A second volume of 300-500 μl of blood collected at euthanasia were processed to serum, aliquoted into small vials and flash frozen in liquid nitrogen for further tests including liver function and lipid tests as per study protocol. Remaining serum was stored frozen for further possible testing.

Liver was collected unless directed otherwise. An outline of the study by week is shown in FIG. 4B.

### Materials and Methods

### Vehicle and Test Article Preparation

a. Vehicle (0.1% CMC with 0.01% Tween 80) were prepared once during the study or as often as needed. Formulation of test articles in vehicles were conducted weekly or as often as needed. At the end of each week, unused formulations were discarded. Formulated test articles were considered stable in the selected vehicles for up to 1 week.
b. Concentrations of test articles were not adjusted for purity.
Formulated vehicle controls and test articles were stored sealed at room temperature (˜25° C.) and protected from light.
   c. 0.1% CMC with 0.01% Tween 80 Vehicle (Dosing for G1 and G2) were prepared as follow:
In a clean and labeled manufacturing vessel or beaker, weigh and add the appropriate amount of water. With a magnetic stirbar and stir plate, begin mixing at approximately 500 rpm. Measure and add the Tween 80.
Continue mixing until homogeneous, and maintain speed of 500 rpm. Slowly add the CMC. Continue mixing until all polymer is completely hydrated. Once completely hydrated, stop mixing.
Store vehicle solution in a sealed vessel at controlled room temperature (approximately 25° C.).
   d. Olive Oil Vehicle (Dosing for G2)
Used as received, no preparation was needed
   e. 80 mg/mL Rifaximin in 0.10% CMC with 0.01 Tween 80 (Dosing for G3, G7, G8 and G9) was prepared as follow:
In a clean and labeled manufacturing vessel or beaker, insert a clean and appropriately sized stir bar. Dispense 0.10% CMC with 0.01% Tween 80 vehicle at the desired volume into the vessel.
Using a stir plate, begin stirring the vehicle at approximately 500 rpm. With continuous mixing, add the calculated amount of rifaximin to achieve target concentration. Continue stirring until a homogenous suspension is achieved.
   f. 10 mg/mL methylnaltrexone bromide in 0.10% CMC with 0.01 Tween 80 (Dosing for G4) was prepared as follow:
In a clean and labeled manufacturing vessel or beaker, insert a clean and appropriately sized stirbar. Dispense 0.10% CMC with 0.01% Tween 80 vehicle at the desired volume into the vessel.
Using a stir plate, begin stirring the vehicle at a speed of approximately 500 rpm. With continuous mixing, add the calculated amount of methylnaltrexone bromide to achieve target concentration. Continue stirring until a homogenous suspension is achieved.
   g. 30 mg/mL curcumin in olive oil (Dosing for G5 and G9) was prepared as follow:
In a clean and labeled manufacturing vessel or beaker, insert a clean and appropriately sized stir bar. Dispense olive oil vehicle at the desired volume into the vessel.
Using a stir plate, begin stirring the vehicle at approximately 500 rpm. With continuous mixing, add the calculated amount of curcumin to achieve target concentration. Continue stirring until a homogenous suspension is achieved.
   h. 20 mg/mL vitamin E in olive oil (Dosing for G6 and G8) was prepared as follow:
In a clean and labeled manufacturing vessel or beaker, insert a clean and appropriately sized stir bar. Dispense olive oil vehicle at the desired volume into the vessel.
Using a stir plate, begin stirring the vehicle at approximately 500 rpm. With continuous mixing, add the calculated amount of vitamin E to achieve target concentration. Continue stirring until a homogenous mixture is achieved.
   i. 10 mg/mL methylnaltrexone bromide and 80 mg/mL rifaximin in 0.10% CMC with 0.01 Tween 80 (Dosing for G7) was prepared as follow:
In a clean and labeled manufacturing vessel or beaker, insert a clean and appropriately sized stir bar. Dispense 0.10% CMC with 0.01% Tween 80 vehicle at the desired volume into the vessel.
Using a stir plate, begin stirring the vehicle at approximately 500 rpm. With continuous mixing, add the calculated amount of methylnaltrexone bromide to achieve target concentration. Continue stirring until a homogenous suspension is achieved.
With continuous mixing, add the calculated amount of rifaximin to achieve target concentration. Continue stirring until a homogenous suspension is achieved.

### 1) Body Weight Monitoring

a. The mice were monitored for body weight on a weekly basis. Body weight was collected on a Melter Toledo scale (500 g) and monitored with twice daily non-invasive visual inspection. A weight log was kept in a study notebook.

### 2) Oral Gavage

a. On each morning, vehicle and test articles were mixed at room temperature using a magnetic stirrer set at medium speed for at least 30 minutes prior to dosing. During the dosing procedure, formulations were in constant stirring to avoid precipitation. Formulations for dosing were drawn from the middle of the container solution.
b. In summary, mice were grasped at the scruff of the neck and the body is held straight. The head was then gently tilted back so the chin was pointed up and aligned with the body. A specially designed “gavage needle” (20-22-gauge X1″) was inserted through the mouth (either side of incisors) and esophagus into the stomach and a maximum of 0.01 ml/g was infused.
c. Intermittent monitoring for up to 1-hour post-procedure was performed for adverse signs. Abnormal signs include but are not limited to: labored respiration, red oral and/or oral nasal discharge, swelling of the axillary region, hunched posture, rough hair coat and/or recumbency.
d. A gavage sheet was maintained and included in the study notebook. A standard concentration for each test article was made as presented above, and a variable volume were gavaged based on individual animal weekly body weight.

### 3) Leaky Gut Assay

a. The leaky gut assay was performed at week 12 (one month on treatment) and immediately prior to sacrifice.
b. The procedure for the dextran was as follow:
   i. 2 g of 4 kD dextran was weighed into a static free weigh dish.
   ii. The dextran was mixed into a 10 ml of PBS solution.
   iii. Between 0.2 ml and 0.4 ml of dextran solution was orally gavaged into mice. Each mouse staggered by 10 minutes to maintain blood dextran consistency during blood collection.
   iv. 4 hours post gavage lidocaine applied to the tails of the mice in the order that they were gavaged.
   v. The mice were lightly restrained in a mouse restraint device.
   vi. After 5 minutes 70% alcohol was applied to a cotton swab and used to wipe off the lidocaine.
   vii. Sterile scissors were used to perform a tail nick.
   viii. 140 μl of whole blood was collected into a capillary tube.
   ix. The capillary tubes were spun at 3,000 RPM for 8 minutes.
   x. The capillary tubes were broken at the supernatant line and the serum was blown into an Eppendorf tube and snap frozen in liquid nitrogen.

### 4) Blood Collection

a. Immediately prior to euthanasia a tail nick was performed. Whole blood was used to measure ketones and glucose.
b. Euthanasia—The mice were placed into a clean cage and fasted for 6 hours prior to euthanasia. Blood collection took place during euthanasia via cardiac puncture. The blood was collected into micro-serum separator tubes and processed to serum by centrifugation under 4 degrees Celsius for 15 minutes at 5,000 RMP, aliquoted in 100 μl aliquots, and serum flash frozen. Serum samples were stored frozen at −80° C. until use.

### 5) Euthanasia

a. Mice were administered carbon dioxide by inhalation as prescribed by the American Veterinary Association and exsanguinated. Secondary euthanasia was performed by cervical dislocation.

### 6) Disposition

a. The carcasses were disposed of according to EVMS institutional standards.

### Collection of Biological Samples

### Fecal Samples

Prior to dosing with dextran for the leaky gut assay, fresh fecal pellets were collected from the mice in each cage, placed into RNAlater (ThermoFisher Scientific) and frozen for possible future gut microbiome analysis.

### Liver Tissue Collection and Processing

At necropsy, assessment and photography of gross morphology of liver and overall body condition were recorded on a specimen sheet for each mouse.

For each mouse, liver tissue was collected and liver sample split between snap frozen, RNAlater, and formalin-fixed for making FFPE blocks for histological assessment purposes.

Body weight and liver weight at time of euthanasia were recorded. Any abnormal gross anatomy signs on the body or liver would have been noted. Any mice found dead during the study had a health monitoring sheet attached detailing the findings. The liver was processed into sections.

Liver samples were fixed in 10% neutral buffered formalin for 36-48 hours depending on the size of the sample before being placed into 70% alcohol. The samples were then processed into FFPE blocks. The FFPE blocks were sectioned and three (3) slides were cut per block and submitted for H&E and Sirius Red staining, and subsequent histology analysis. The samples' identity was blinded at the time of shipment for unbiased scoring.

Slide evaluation and scoring include NAS component scoring (fibrosis, steatosis grade and percentage, inflammation, ballooning), NAS score, SAF activity score, Fibrosis NASH CRN Score, Perisinusoidal Fibrosis score, and NASH category score. A histopathology sub-report is to be prepared containing scores and representative photographic images from each group.

The remaining of liver tissue samples was either flash frozen or mixed with a volume of RNAlater, and stored frozen at −80° C.

### Gut Contents

Gut contents from cecum and colon will be collected from each mouse at necropsy and frozen in RNAlater at −80° C.

### Blood Collection

Blood was collected via cardiac puncture with a 20-gauge needle at necropsy and placed into a Becton Dickinson serum separator tube. Blood were let to clot cold for 15 mins at 4° C. then spun at 5000 rpm for 15 mins in a Fisher refrigerated centrifuge. The serum was drawn off and aliquoted into Eppendorf cryotubes then snap-frozen and stored in a freezer approximately at −80° C. until pulled and thawed for further testing.

Lipid (total cholesterol, LDL, HDL, triglycerides, HDL calc.) and hepatic (ALB, AST, ALT, Alk, Phos, BUN, GGT, T Bil) panels were performed as described below. Due to natural variations in mice physiology extra serum might not be available to repeat testing.

### Blood Glucose Measurement

At necropsy, one drop of whole blood from the tail snip was placed on a test strip and inserted into the OneTouchPlus Ultra Glucometer for analysis.

### Serum Lipid Profile Measurement

40-50 microliters of thawed serum were placed into the sample well of an AL-10-991 test cassette, then the test cassette placed into the Alere Cholestech LDX blood lipid analyzer. The Alere Cholestech is FDA approved and CLIA waived for human use at POC and is used by numerous drug companies and CROs in preclinical studies because it only requires a small amount of blood. It may be used with whole blood, serum or plasma for humans, but mouse blood clots much faster and so whole blood in our experience has a higher failure rate. We therefore used serum.

Readouts were:
1. Total cholesterol (T-chol)
2. Triglycerides (TRIGS) (45 lower LOD)
3. Glucose (standard range)
4. HDL (100 upper LOD)
5. LDL (calculated T-Chol—HDL)

### Serum LFT Measurement

Abaxis VS2 blood chemistry analyzers were used for measuring LFTs. This instrument is FDA approved for veterinary use, CLIA waived, and self-calibrating. It is the equivalent instrument to the Abaxis Piccolo instrument for human diagnostic testing. Phenobarbital test rotors (catalog #500-0049) were used which measure AST, ALT, ALP, GGT, ALB, and Total Bilirubin. The reactions are colorimetric, read automatically by the VS2 instrument, and results printed on a sticker which is attached to the necropsy specimen sheet and then entered into Excel. A minimum of 100 microliters of sample is needed for this test rotor, which may be either undiluted serum or diluted serum, in a ratio of 1:1 50 microliters of serum: 50 microliters of PBS without affecting accuracy of measurement of analytes. Significant hemolysis may render samples un-readable.

### Results

### Body Weight

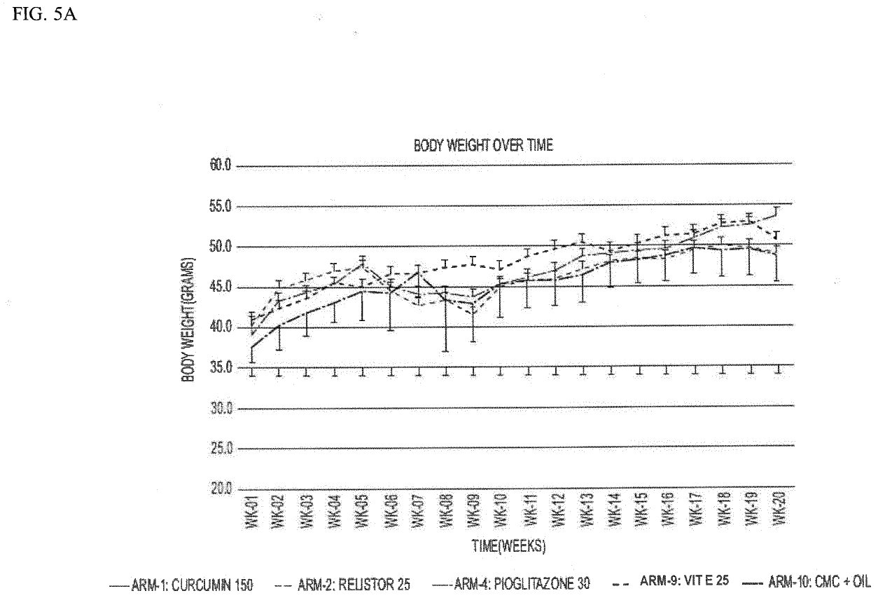

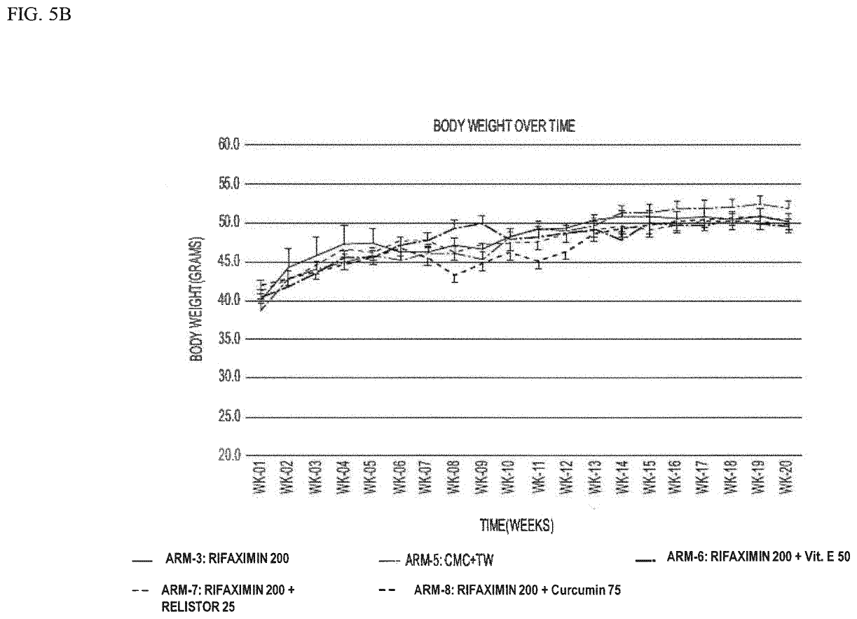

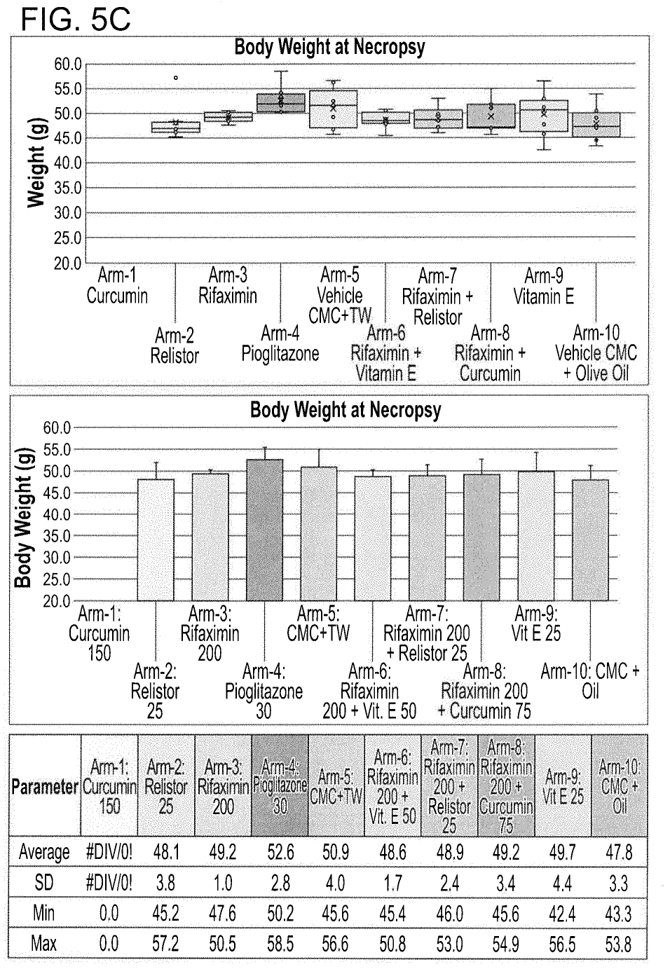

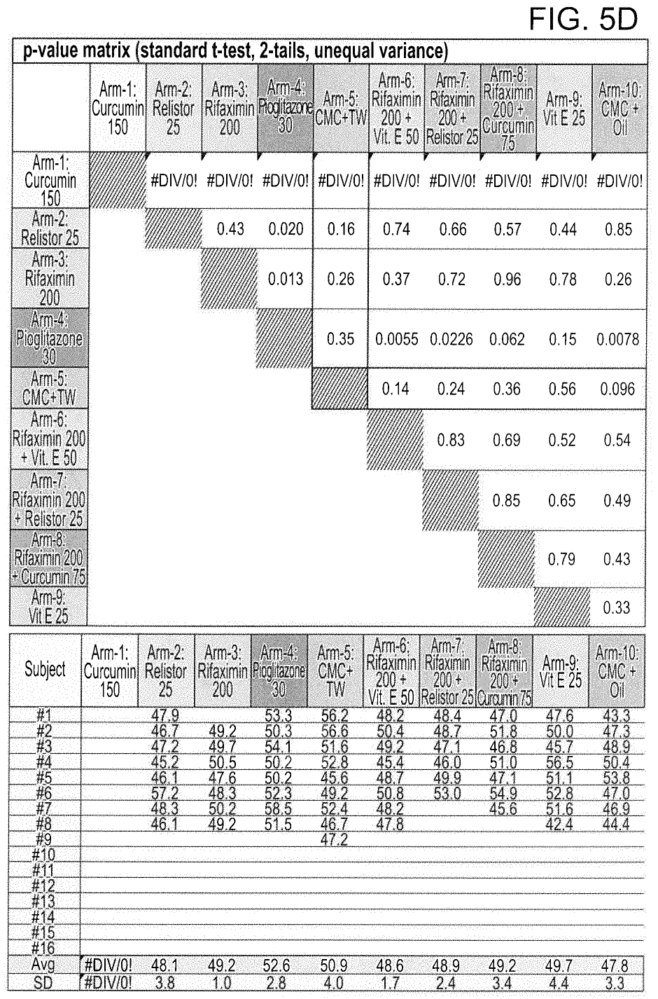

All on the WDSW diet gained weight within the expected range and timeframe. The weight in WDSD-fed mice, regardless of the treatment were not significantly different throughout the study timeframe. However, in the MNTX and Pioglitazone (FIG. 5A) a transient decrease in body weight was evident albeit before treatment started. Mice in those 2 groups caught up with the other groups and no major significant changes in body weight were recorded between arms.

At the time of this interim report, there was no data for Arm-1 (Curcumin-only) and a symbolic weight was added for illustration purposes until relevant data is available.

FIG. 5A shows the animal body weight for study arms/groups 1, 2, 4, 9 and 10 throughout the duration of the study (in weeks).

FIG. 5B shows the animal body weight for study arms/groups 3, 5, 6, 7 and 8 throughout the duration of the study (in weeks).

At necropsy (see FIGS. 5C-5D), there were no significant differences between treatment groups for the body weight. All groups receiving WDSD diet had significantly higher body weight at necropsy when compared to the mice group fed the normal chow diet.

### Liver Weight

Liver weights in the DIAMOND™ mouse model tend to range between 4-5 grams for a mouse on WDSW diet without tumor development. Hepatic steatosis (fat in the liver), fibrosis (collagen scar deposits) and inflammation (cause higher fluid content) increase liver weight.

MNTX (Arm-2) was associated with a significantly lower liver weight vs Vehicle Control (Arm-5). Rifaximin alone did not reduce liver weight vs Arm-5 but its combination with other agents (Vit. E, MNTX or Curcumin) did.

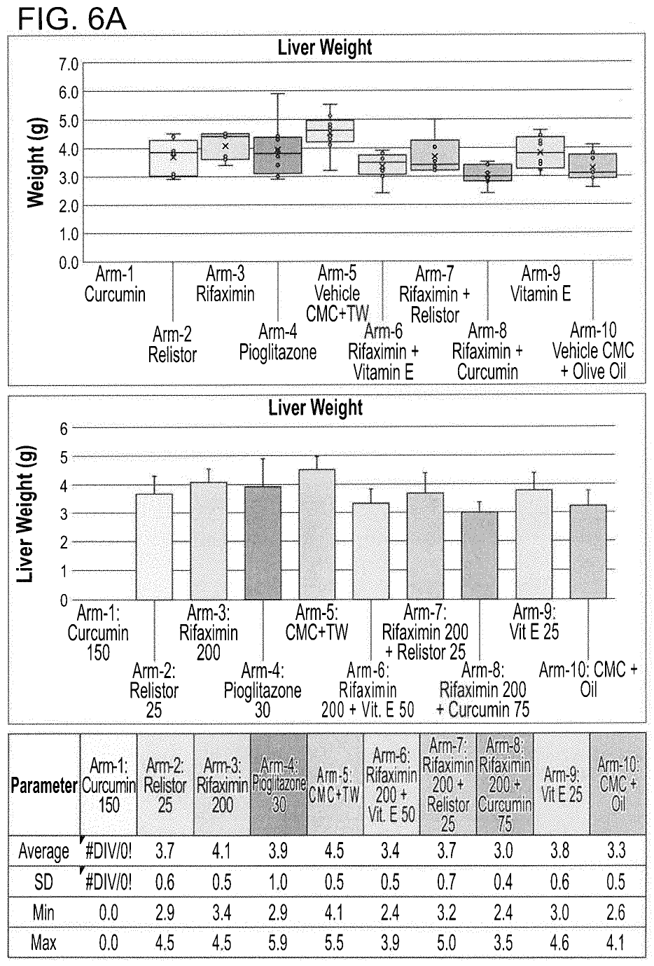

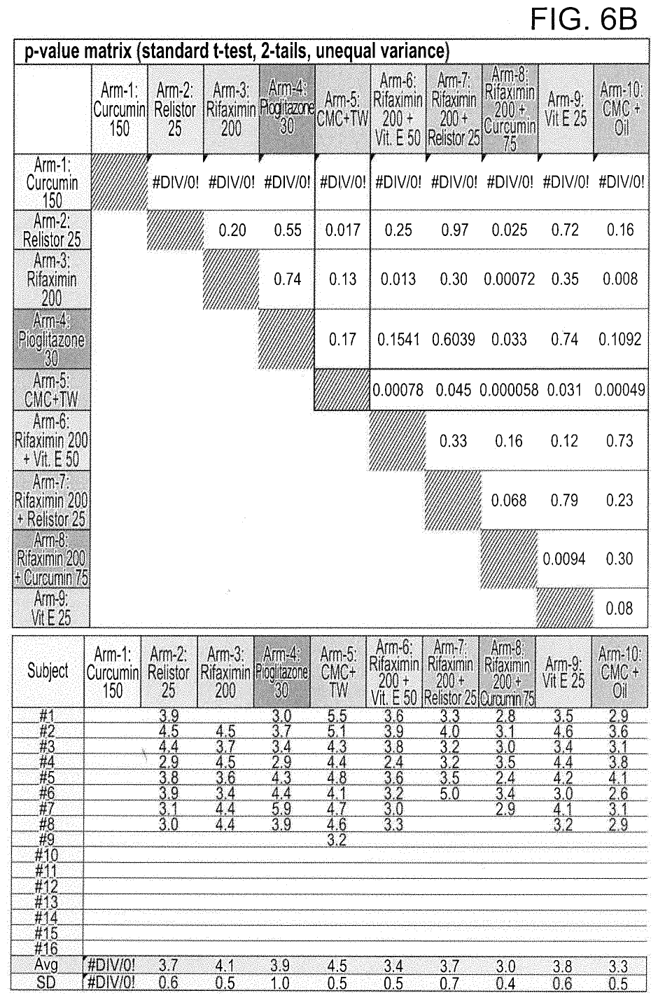

FIGS. 6A-6B shows the liver weight at necropsy.

### Glucose

Glucose levels were relatively stable between the groups when compared to the vehicle control groups (Arm-5 or Arm-10). Rifaximin combination had a trend toward a lower glucose level although most of those were non-significant.

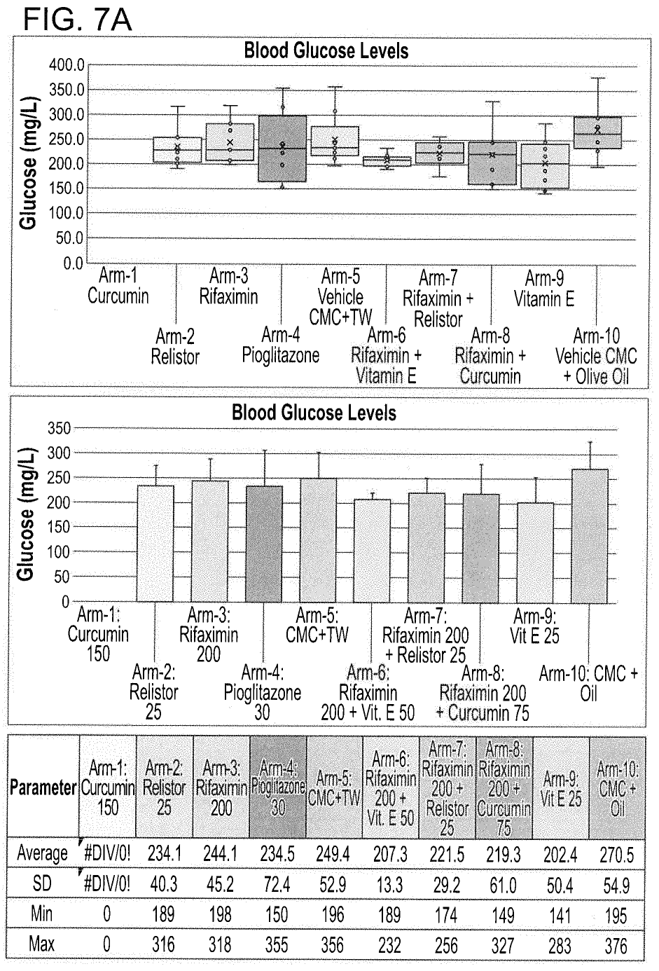

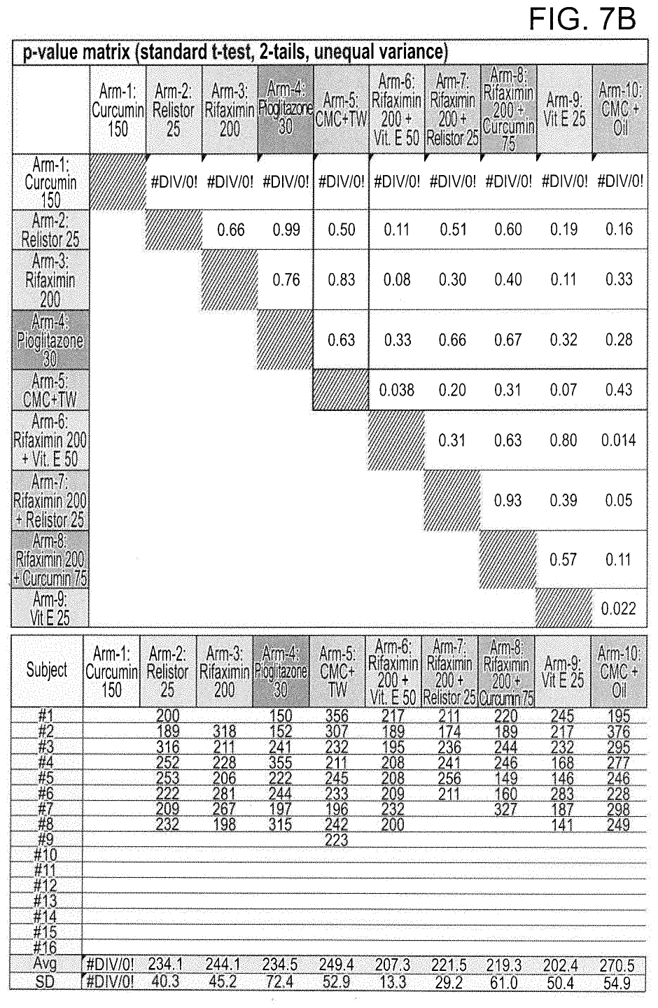

FIGS. 7A-7B shows the glucose blood levels.

### Ketones

Rifaximin levels were relatively similar to those in the Arm-5 vehicle control groups with a few exceptions. Pioglitazone and Rifaximin decreased the levels of ketones while Vitamin E moved ketone levels up. However, Rifaximin combinations were not significantly different; Rifaximin+Vit. E (Arm-6) had essentially the same ketones levels as in the vehicle control (Arm-5) due to opposing effects of those 2 agents. Addition of MNTX to Rifaximin (Arm-7) blunted any response of the ketones to Rifaximin alone.

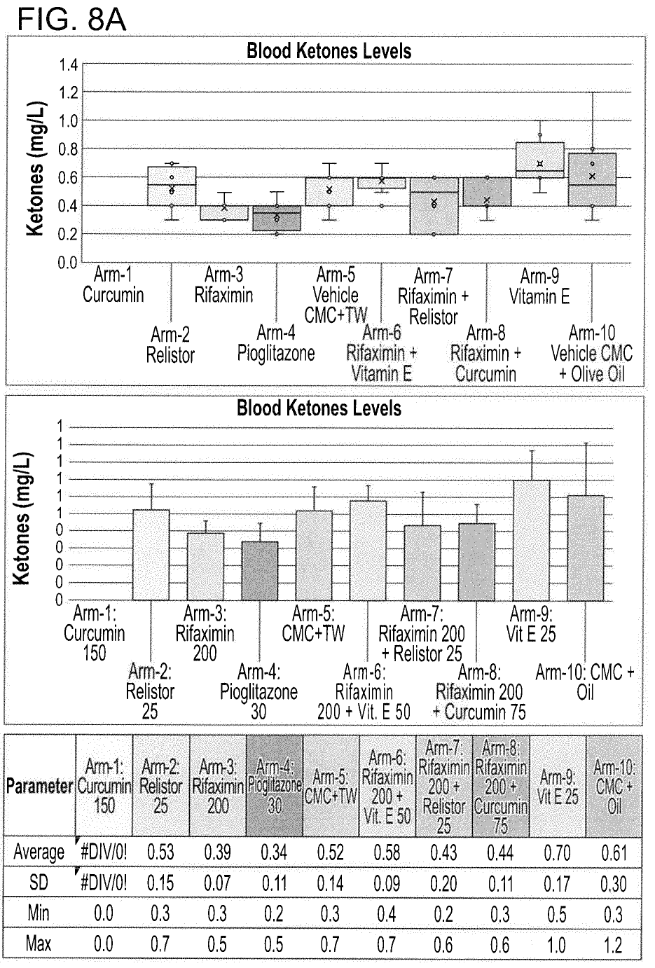

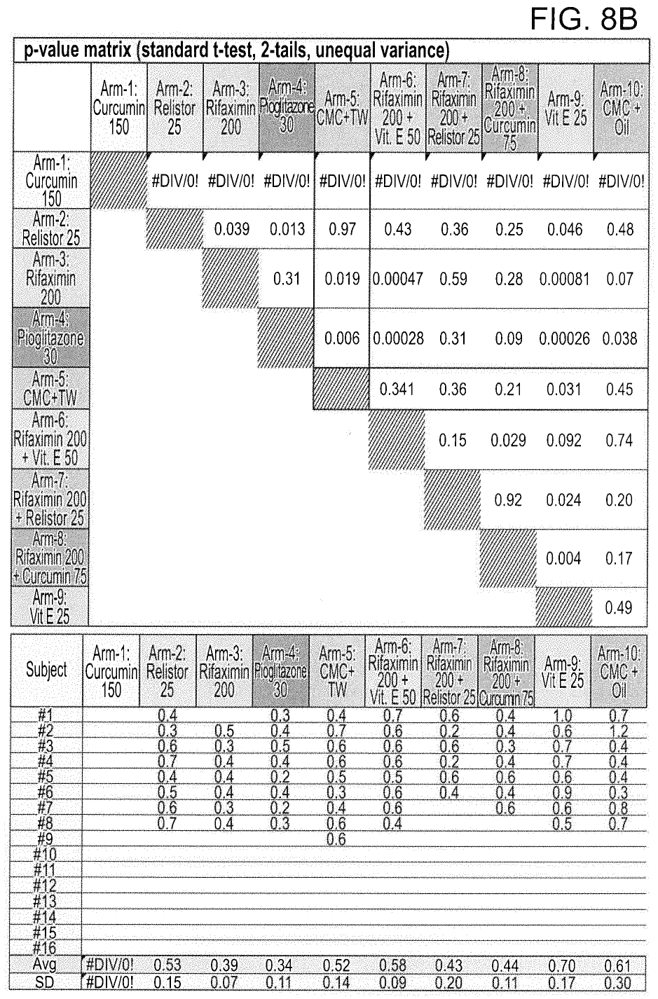

FIGS. 8A-8B shows ketones blood levels.

### Cholesterol

When compared to the vehicle control groups (Arm-5 or Arm-10), none of the treatment groups were associated with a statistically significant change in cholesterol. Between treatment groups, some changes were noted but their biological relevance remains to be established given the absence of response vs the vehicle control arms.

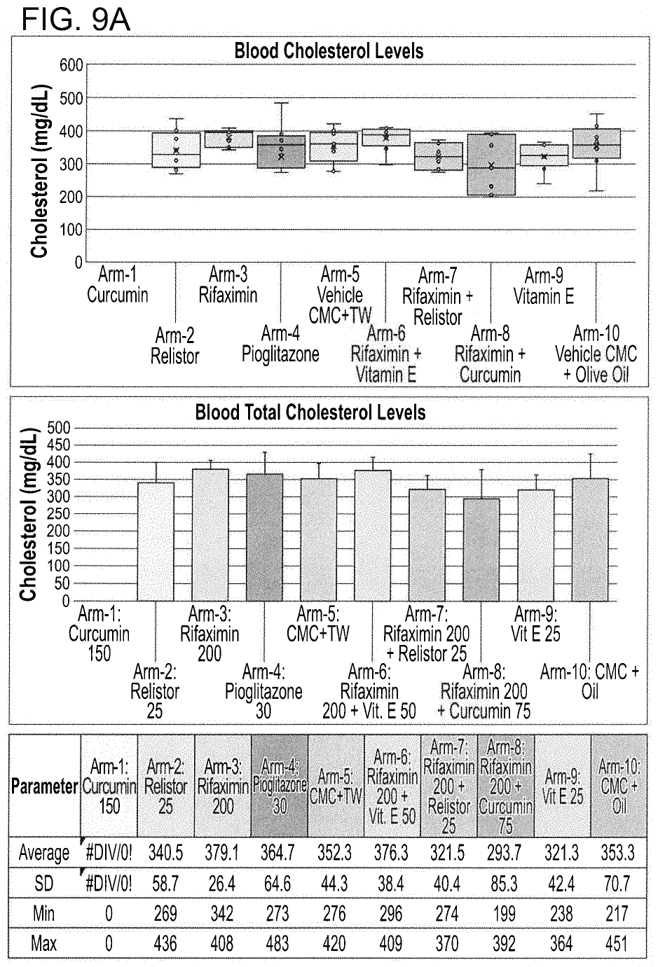

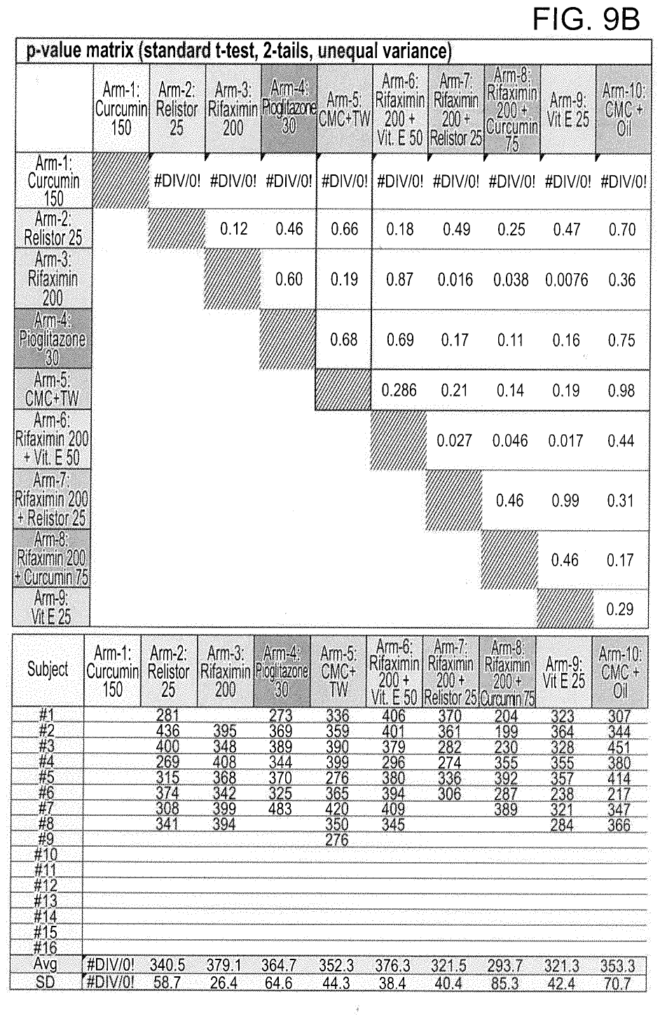

FIGS. 9A-9B shows cholesterol blood levels.

### Triglycerides

Pioglitazone (Arm-4) significantly decreased the levels of triglycerides vs vehicle control (Arm-5) while other monotherapy treatments (Arm-2, Arm-3 or Arm-9) had no significant effect. While Rifaximin and Vitamin E alone had no effect, their combination (Arm-6) significantly increased triglycerides. The same is true when Rifaximin and Curcumin (Arm-8) are combined. Note: In the Rifaximin+Vitamin E arm (Arm-6) one outlier value was removed for mouse #021; its triglycerides value was 426 mg/dL.

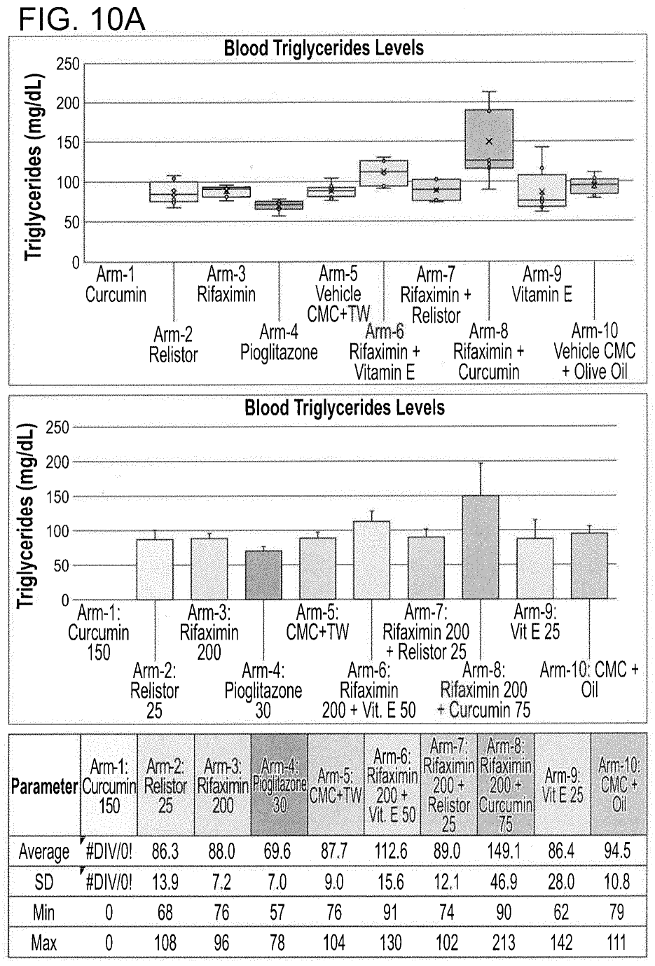

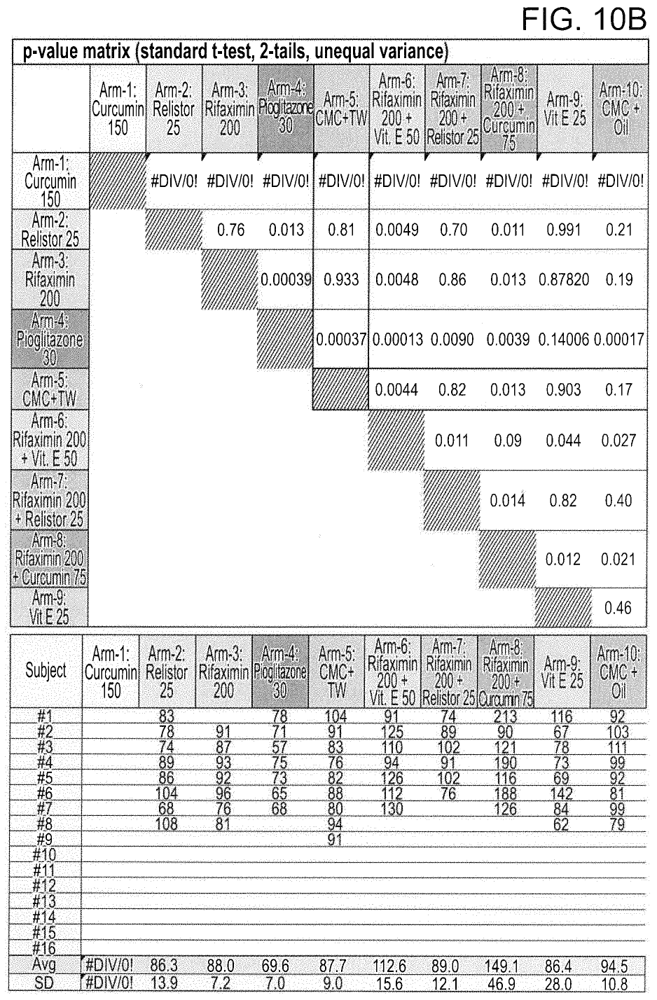

FIGS. 10A-10B shows triglycerides blood levels.

### Alanine Aminotransferase (ALT)

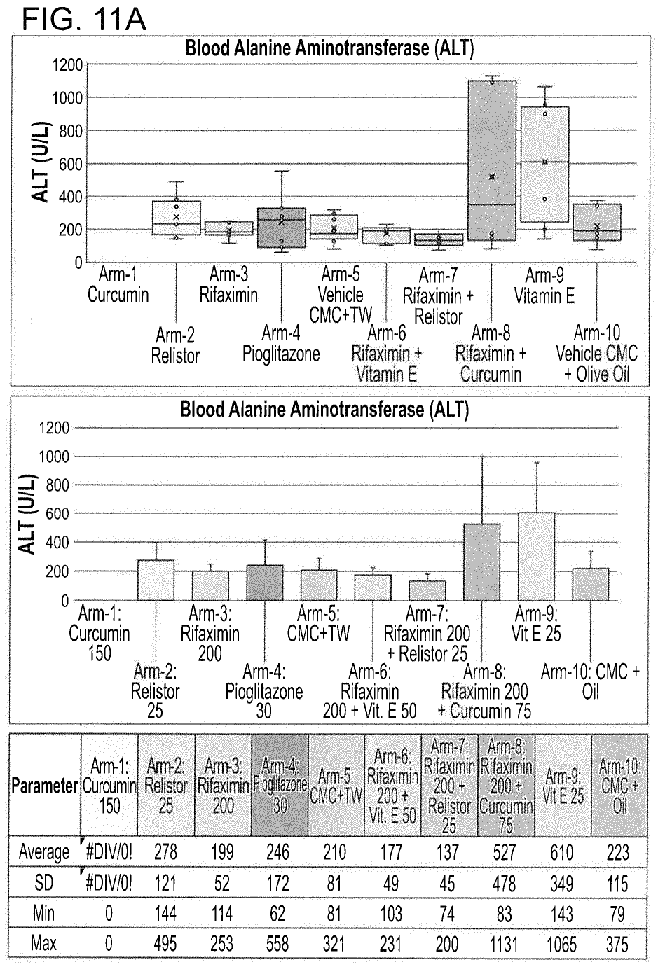

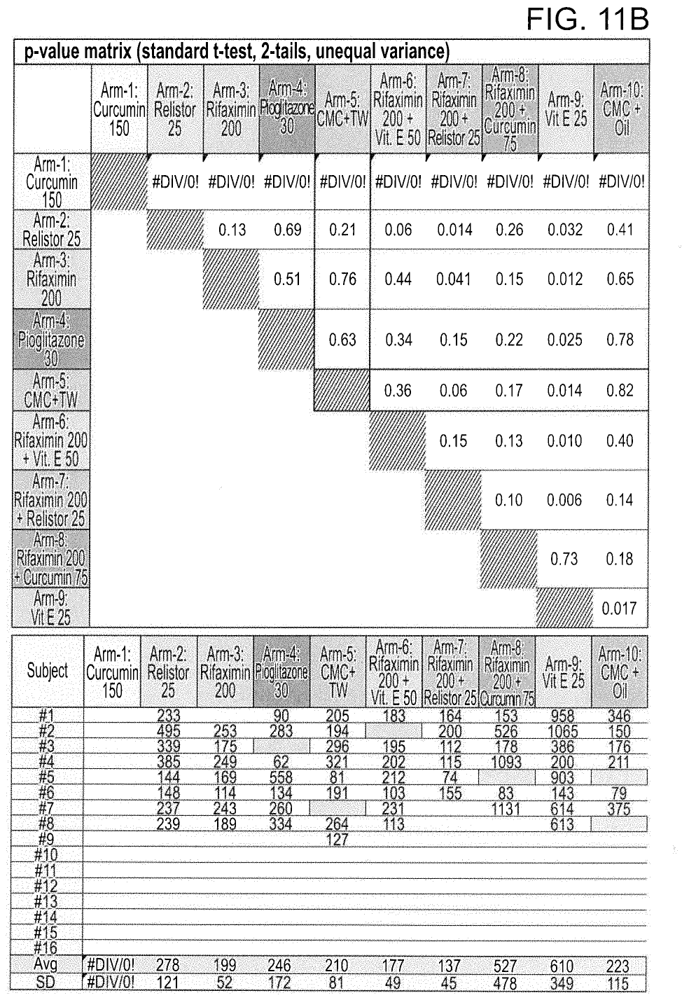

Overall, treatments did not significantly change ALT levels in most arms vs control vehicles. A high variability in ALT is noted in the combination group with curcumin and the Vitamin E group (the latter associated with multiple significant changes. Note: Four mice had Hemolysis levels that warranted removal of their ALT values (i.e., #7 in Arm-5; #5 in Arm-8; #5 in Arm-10; and #8 in Arm-10 as listed in FIG. 11B table) and two mice had outlier ALT values; removal of those value did not change the overall interpretation (i.e., #3 in Arm-4 and #2 in Arm-6 as listed in FIG. 11B table).

FIGS. 11A-11B shows ALT blood levels.

### Alkaline Phosphatase (ALP)

Overall, treatments did not significantly change ALP levels in most arms vs control vehicles; Pioglitazone (Arm-4) was borderline significant with a small increase in ALP vs the vehicle control arm (Arm-5).

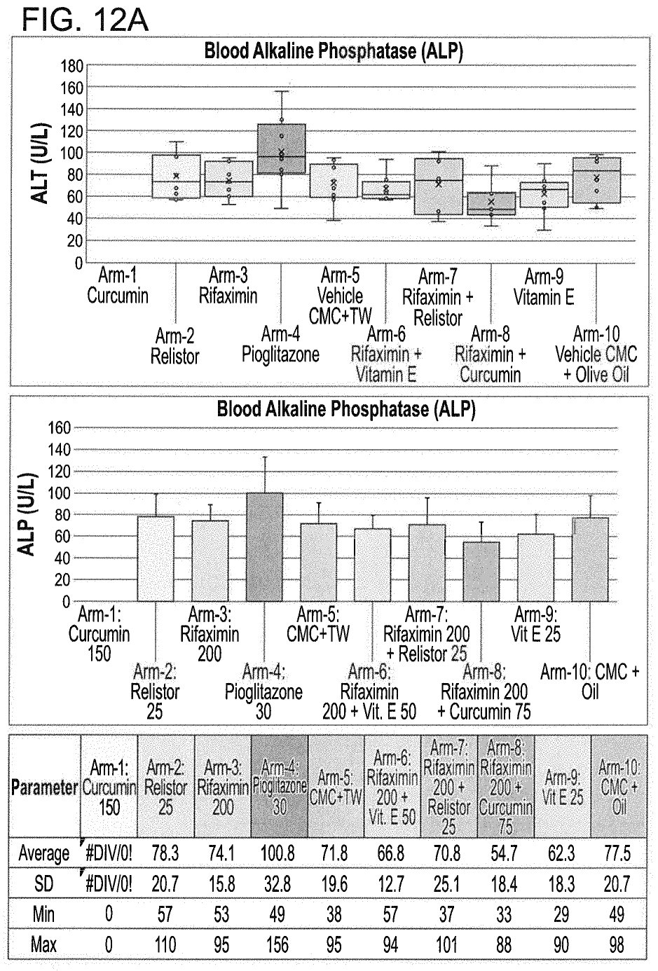

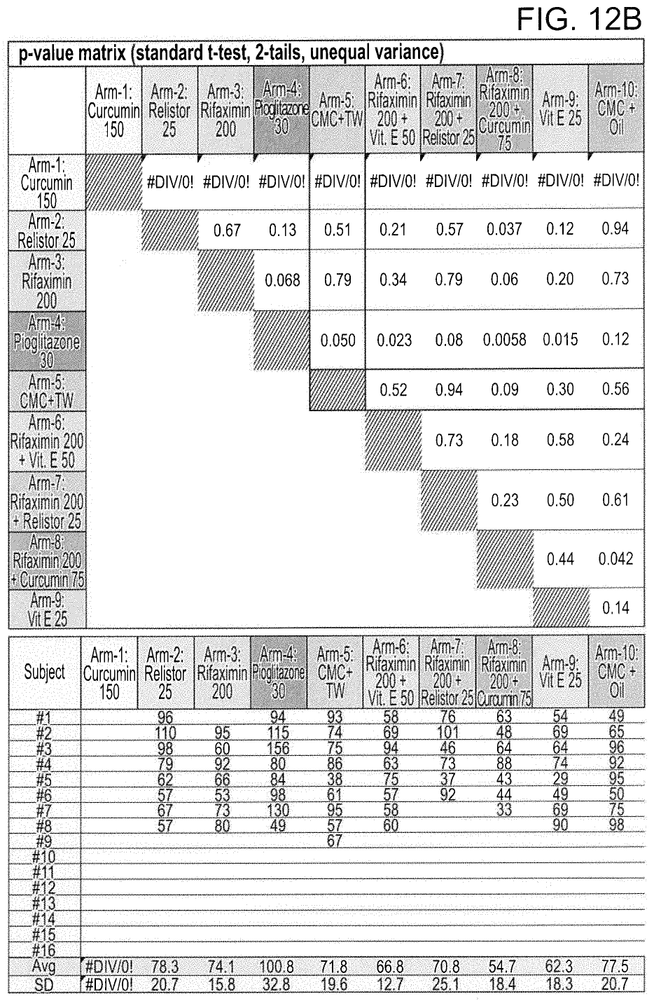

FIGS. 12A-12B shows ALP blood levels.

### Aspartate Aminotransferase (AST)

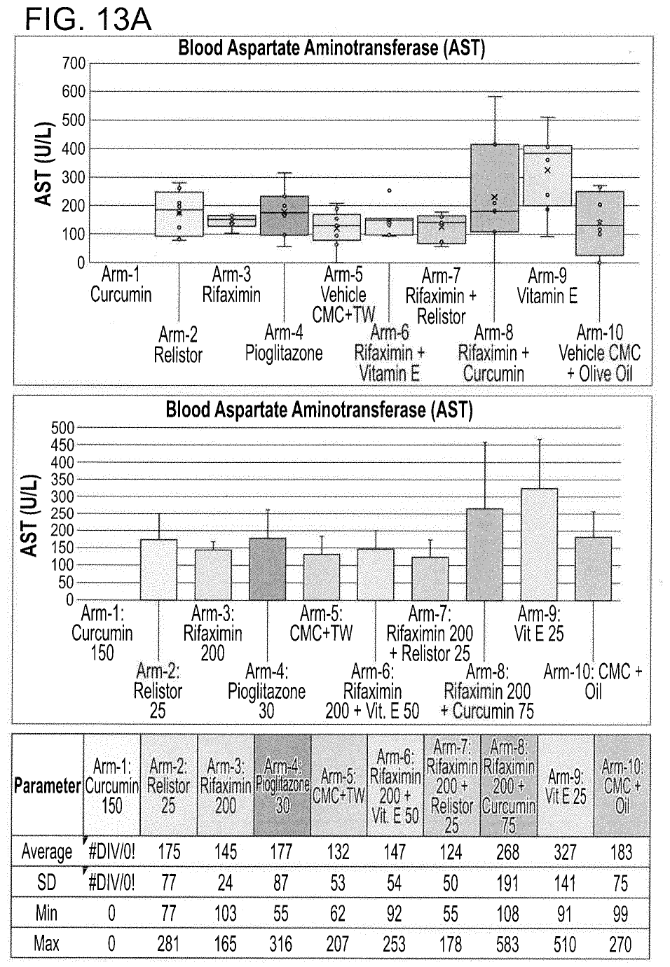

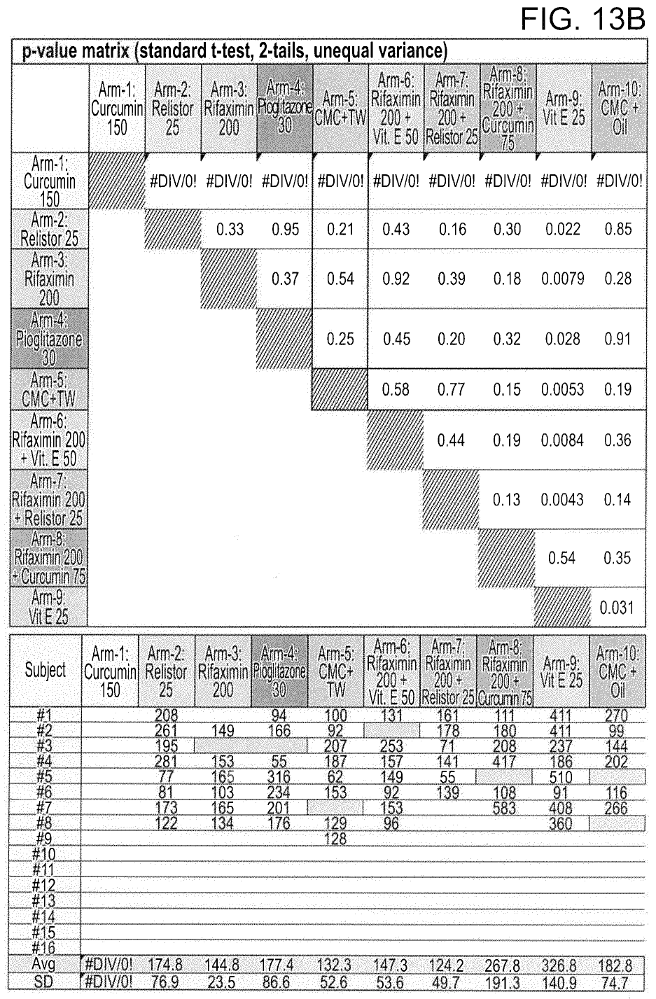

Overall, treatments did not significantly change AST levels in most arms vs control vehicles and the results for AST are very similar to those for ALT; high variability in combination arm with curcumin and in the Vitamin E arm (associated with multiple significant changes). Note: Four mice had Hemolysis levels that warranted removal of their AST values (i.e., #7 in Arm-5; #5 in Arm-8; #5 in Arm-10; and #8 in Arm-10 as listed in FIG. 13B table) and three mice had outlier AST values (i.e., #3 in Arm-3; #3 in Arm-4; and #2 in Arm-6 as listed in FIG. 13B table); two of the latter are the same outliers as for ALT.

FIGS. 13A-13B shows AST blood levels.

### Albumin

Albumin was stable from one arm to the other with no significant changes except for a few in the Vitamin E arm (e.g., lower albumin in Vitamin E (Arm-9) vs Vehicle control arm (Arm-10). Note that mouse #039 in Arm-10 (Vehicle control with Olive Oil) was an outlier. However, removing this data point did not change the interpretation.

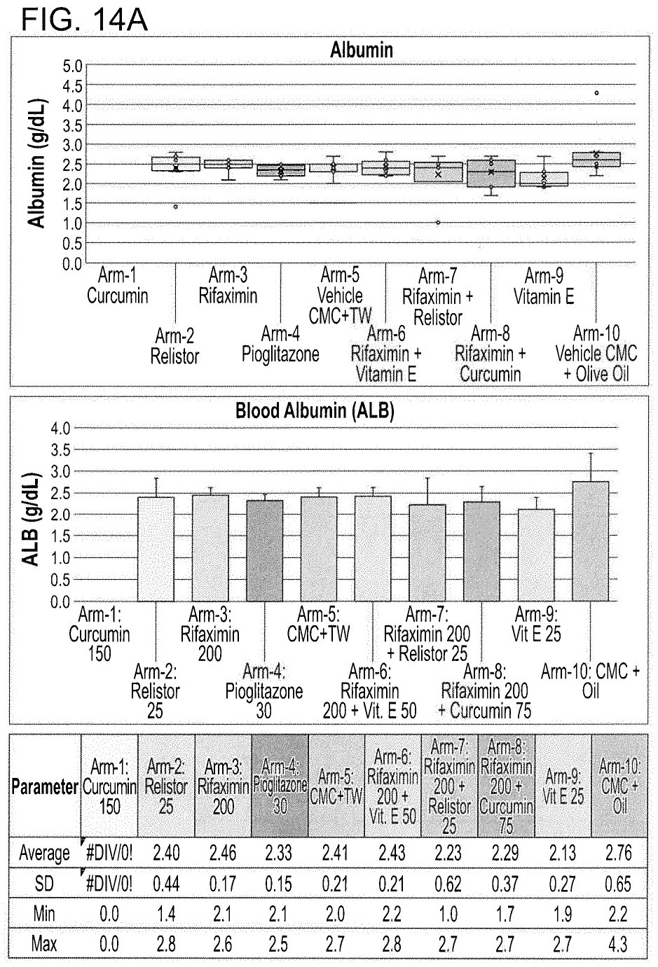

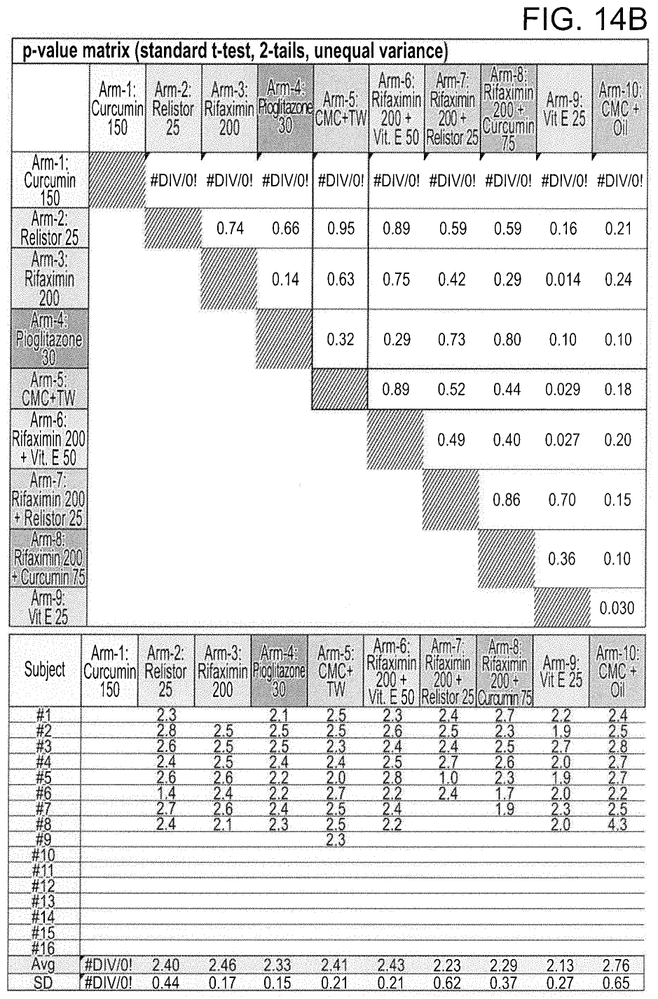

FIGS. 14A-14B shows albumin blood levels.

### Blood Urea Nitrogen

Several significant changes in BUN occurred in the different arms of this study when compared to the Vehicle control groups (Arm-5 and Arm-10). Pioglitazone (Arm-4) significantly increased BUN. The same is true for Rifaximin alone or in combination with Curcumin or Vitamin E but not in combination with MNTX. Note that mouse #039 in Arm-10 (Vehicle control with Olive Oil) was an outlier but removing this data point did not change the interpretation.

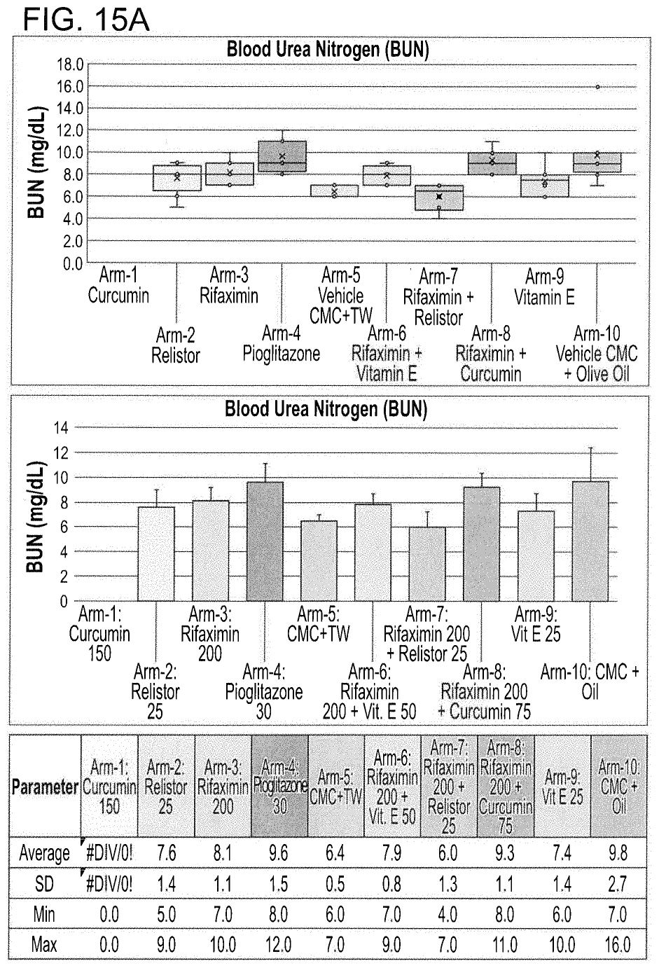

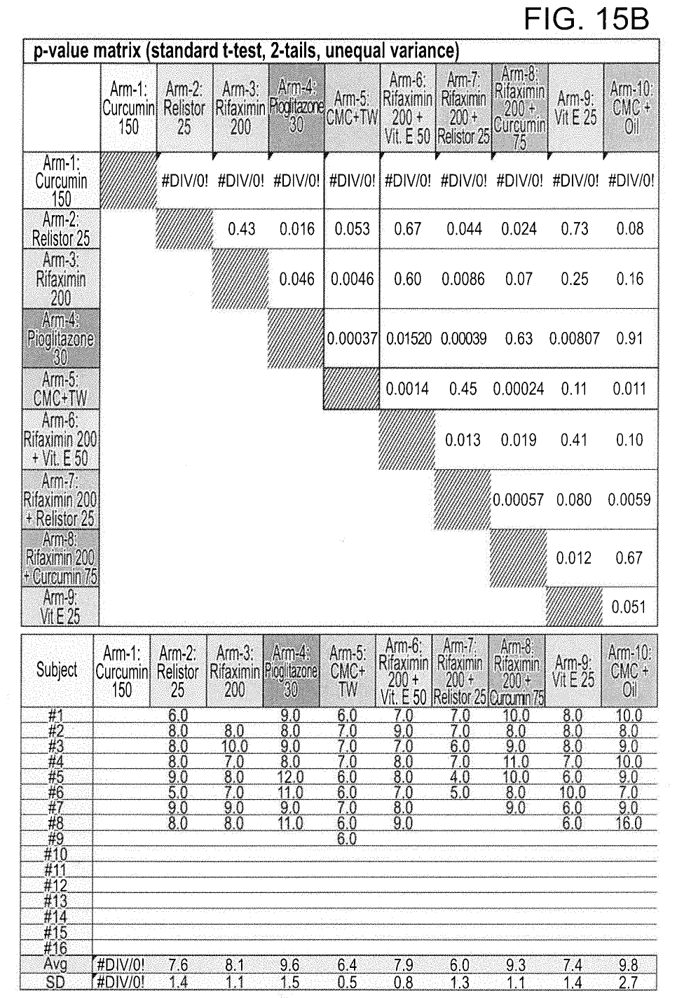

FIGS. 15A-15B shows blood urea nitrogen levels.

### Dextran Assay—Gut Permeability

Some interesting observations and data interpretations were possible from this study that included a Dextran Assay/Gut Permeability (GP) data at two time points; one at week-12 after 4 weeks of treatment and a second one at week-20 after 12 weeks of treatment.

At week-12, very few differences in GP were noted between the study arms (see FIGS. 3A-3B). The MNTX arm (Arm-2) was the only arm displaying a significantly lower GP when compared to the vehicle control (Arm-5).

At week-20, several significant changes were noted between the different study arms (see FIGS. 3C-3D):

### MNTX Arm (Arm-2):

The significantly lower GP noted at week-12 for MNTX persists at week-20 suggesting a sustained effect of MNTX on GP.

### Rifaximin Arm (Arm-3):

Whereas no changes were noted at week-12, by week-20 there was a significantly lower GP in that arm when compared to the vehicle control (Arm-5). This suggests that the effect of Rifaximin on GP develops in time; a finding consistent with the fact that Rifaximin is an antibiotic whose effectiveness develop over time rather than instantly as might be the case for MNTX.

### MNTX+Rifaximin Arm (Arm-7):

The combination of MNTX and Rifaximin tends to be associated with the lowest GP although the actual GP value for Arm-7 is not statistically lower than in the MNTX-only or Rifaximin-only arms (Arm-2 and Arm-3, respectively). This suggests that the combination does not interfere with the effectiveness of either or both drugs.

Rifaximin in Combination with Vitamin E or Curcumin (Arm-6 and Arm-8, Respectively):

There was no statistically significant difference s in these groups when compared to the vehicle control groups (Arm-5 and Arm-10). This suggests that these two compounds blunt the effectiveness of Rifaximin.

### Pioglitazone Arm (Arm-4):

There is a significantly lower GP in this arm at week-20; the size effect is no greater than that for MNTX, Rifaximin or their combination. This effect is consistent with the literature and it is believed Pioglitazone mechanism of action on colonic permeability happens through its anti-inflammatory properties.

### Vitamin E Arm (Arm-9):

Similar to the results at week-12, Vit. E alone has no effect on GP, although it seems to blunt the effect of Rifaximin when combined with that drug. Continuous administration of Vit. E trends toward an increase in GP although it did not reach statistical significance. However, this trend may explain the “blunting” effect Vit. E has on Rifaximin.

### Week-20 vs Week-12.

The main findings are as follow (see FIGS. 3D-3E):
MNTX's effect is maintained but does not increase (or lost) over time.
Several trends toward lower GP were noted but none are statistically significant although the trend for Rifaximin-alone (Arm-3) is borderline significant (p=0.051)
Trends toward higher GP were noted too but none are close to statistical significance

### 12-Week Dextran Assay

At week-12, after only 4-weeks of treatment (start at week-8), very few differences in GP were noted between arms. Only MNTX alone seems to be associated with a significantly lower GP when compared to the vehicle control (Arm-5).

FIGS. 3A-3B shows 12-week Dextran Assay/Gut Permeability.

### 20-Week Dextran Assay

At week-20, after 12 weeks of treatment (start at week 8), several significant changes were noted between the different study arms. When comparing with the vehicle control group

(Arm-5), there were significantly lower GP in the MNTX-only arm (Arm-2), Rifaximin-only arm (Arm-3), the Pioglitazone arm (Arm-4), the MNTX+Rifaximin combination arm (Arm-7) but not in the other study arms.

FIGS. 3C-3D shows 20-week Dextran Assay/Gut Permeability.

### Week-20 vs Week-12 Dextran Assay

This comparison was restricted to those mice with Dextran assay at both time-points (12-week and 20-week) and done using a paired t-test.

The only study arm with a trend toward a lower GP from week-12 to week-20 that is borderline significant is Arm-3 (Rifaximin alone, p-val 0.051). Other groups have trends toward lower GP (Pioglitazone, Rifaximin+MNTX, Rifaximin+Curcumin) although those are statistically not significant.

Trending upward were the Vitamin E-only arm (Arm-9) and the Vehicle Control arm (Arm-5) suggesting in the latter group a deterioration of GP although, again, these trends were statistically not significant.

FIG. 3E-3F shows a comparison between week-12 and week-20-Dextran Assay/Gut Permeability for those mice with dextran assay data at both time points. Bar Graph (upper panel) and basic descriptive statistics and paired t-test (bottom panel).

### Liver Steatosis %

When compared to the control Arm-5 (WDSW+CMC-Tween) as the reference group, the only statistically significant change seen is for a slight increase in steatosis in the Rifaximin Arm (Arm-3).

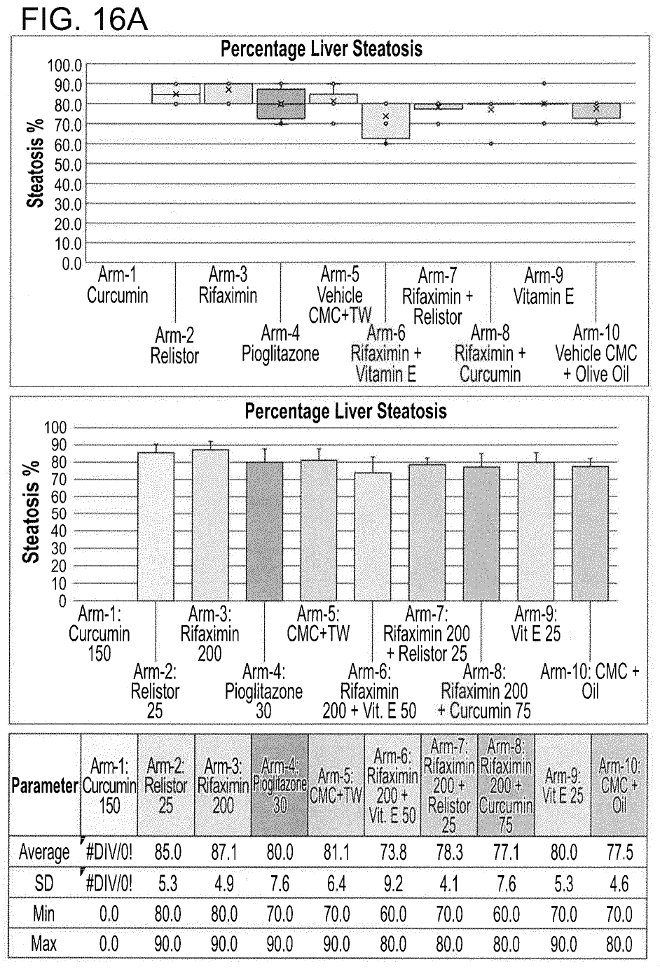

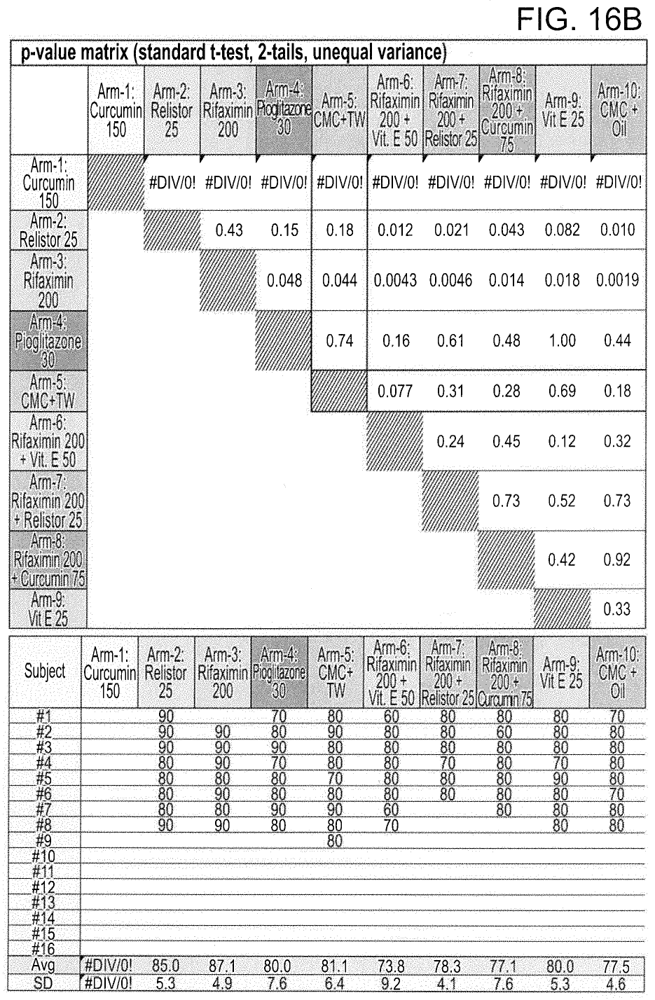

FIGS. 16A-16B shows liver steatosis percentage.

### Liver Steatosis Grade

There were no statistically significant differences between the control arm (Arm-5; WDSW+CMC-Tween) and any of the other groups for Steatosis Grade. This is not surprising since the translation of the percentage steatosis into few categorical steatosis grade classes will tend to nullify small changes with the steatosis percentage.

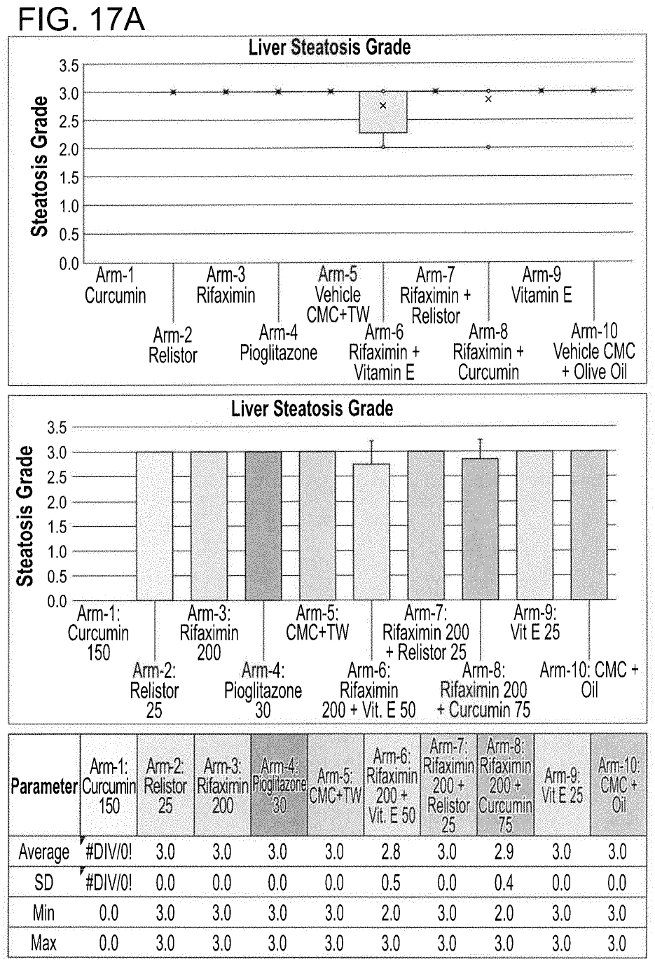

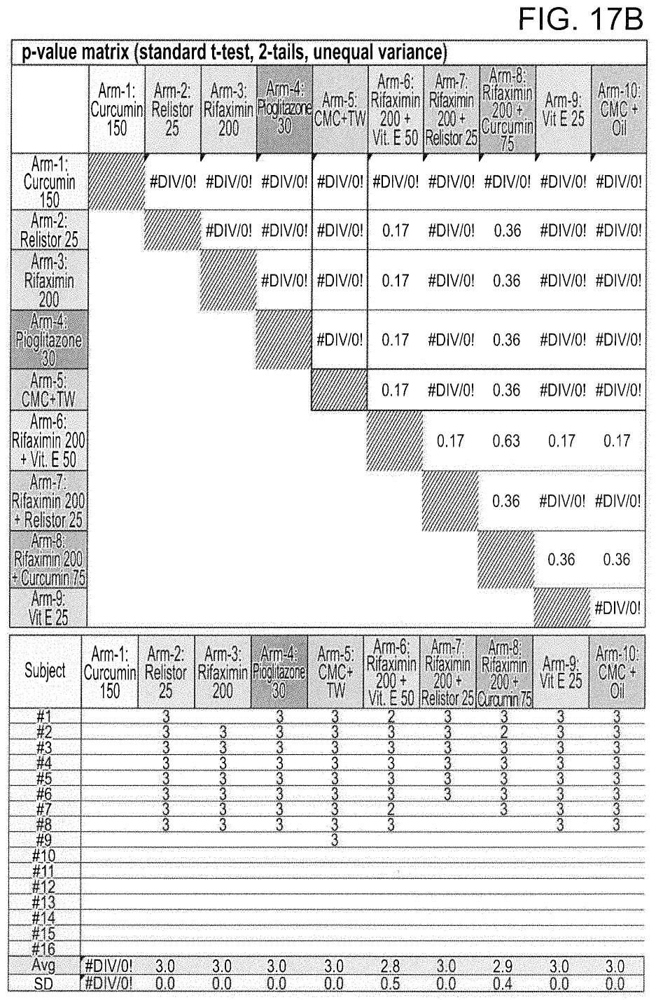

FIGS. 17A-17B shows liver steatosis grade.

### Hepatocyte Ballooning

There were no statistically significant differences between the control arm (Arm-5; WDSW+CMC-Tween) and any of the other treatment groups (and second control Arm−Arm-10: Vehicle+CMC+Olive Oil) for Hepatocyte Ballooning.

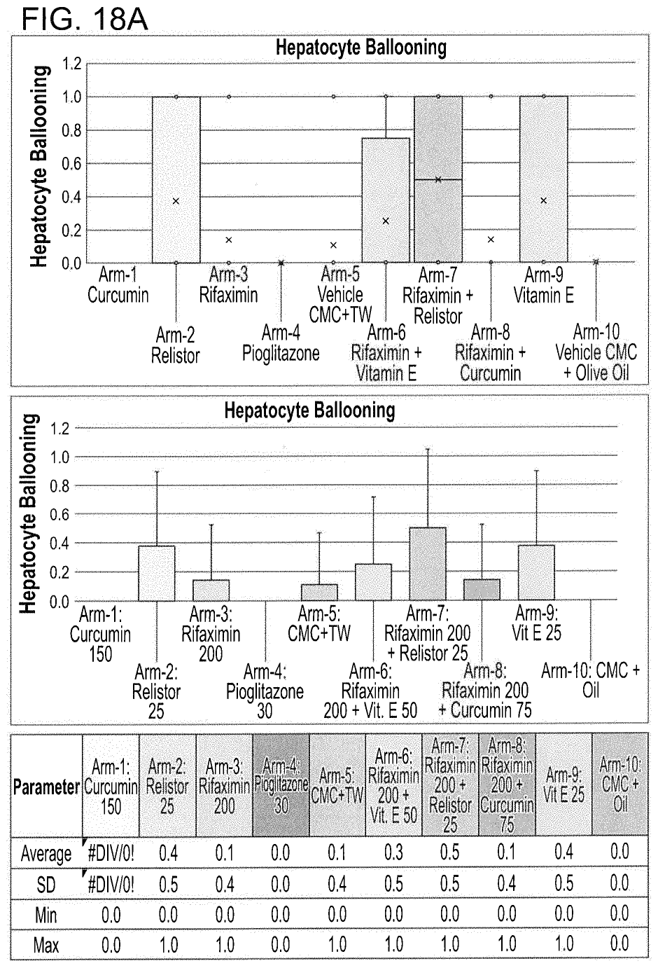

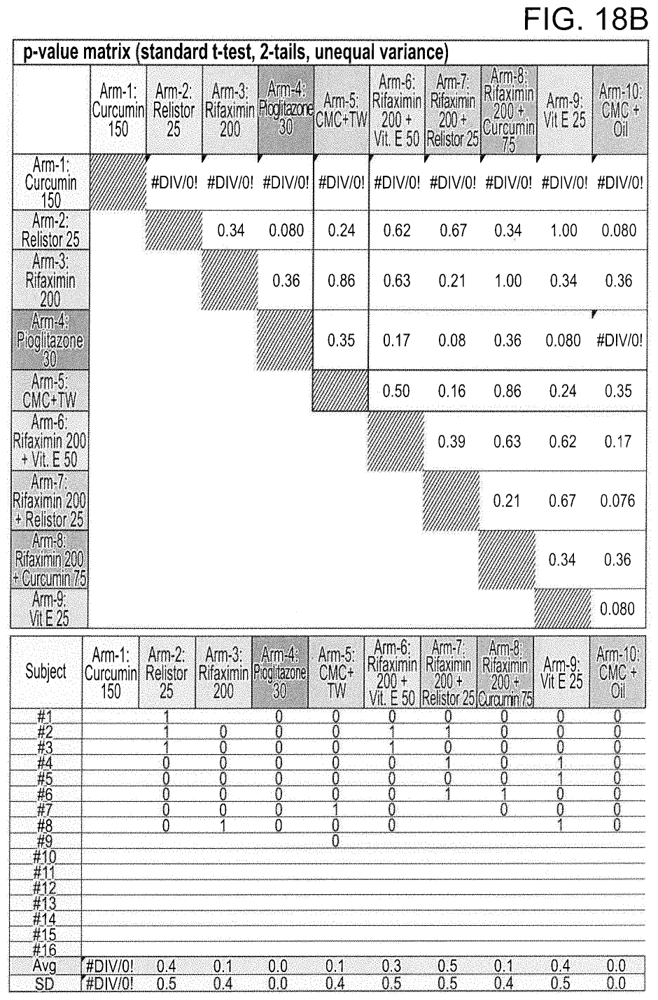

FIGS. 18A-18B shows hepatocyte ballooning.

### Lobular Inflammation

The only statistically significant difference in Lobular Inflammation was for an increase in Arm-9 (Vitamin E, 50 mg) when compared to the control Arm (Arm-5; WDSW+CMC-Tween). Pioglitazone was the only treatment trending toward reducing lobular inflammation (all other treatment groups tended to increase lobular inflammation) although not in a statistically significant way.

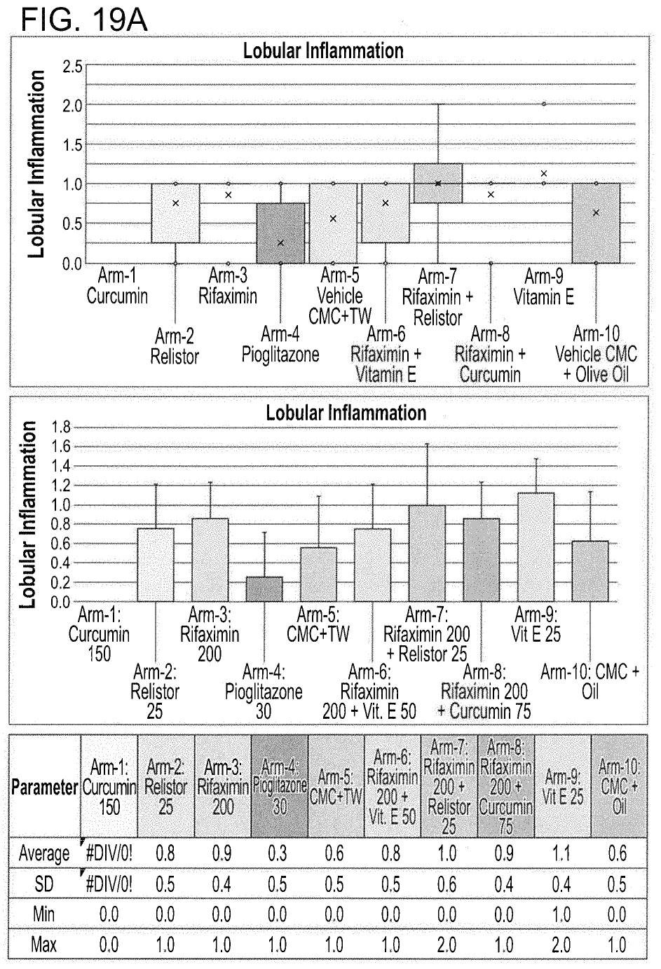

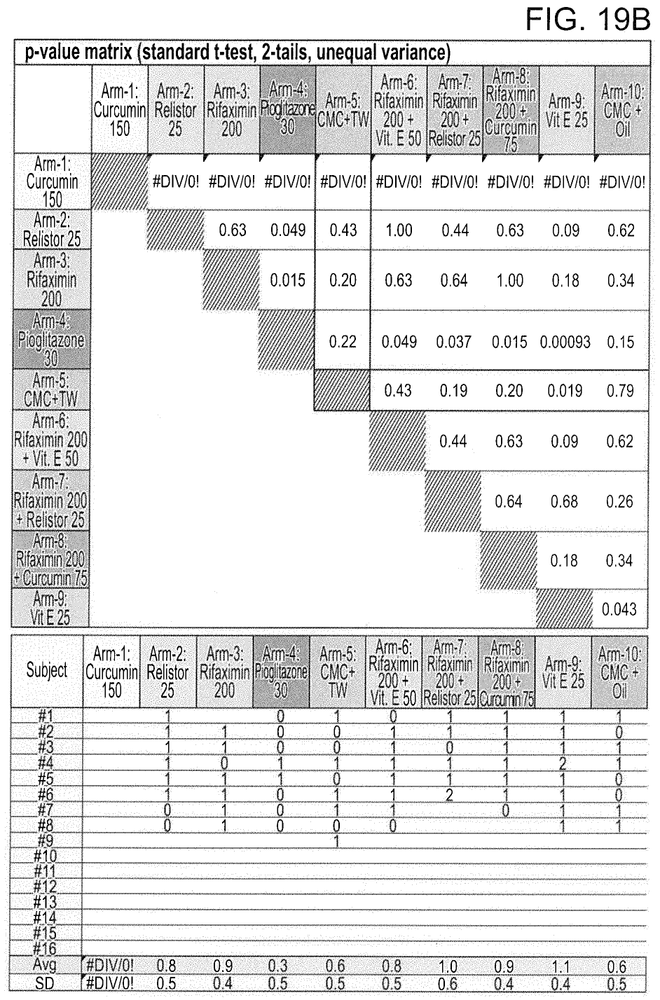

FIGS. 19A-19B shows lobular inflammation.

### NAFLD Activity Score (NAS)

The only statistically significant difference in NAS was for a slight increase in Arm-9 (Vitamin E, 50 mg, p=0.034) when compared to the control Arm (Arm-5; WDSW+CMC-Tween). Pioglitazone was the only treatment trending toward reducing NAS although not in a statistically significant way. It is likely that the decrease in Lobular Inflammation was the driving force behind this decrease in NAS since the profile of changes in NAS mirror those in Lobular Inflammation.

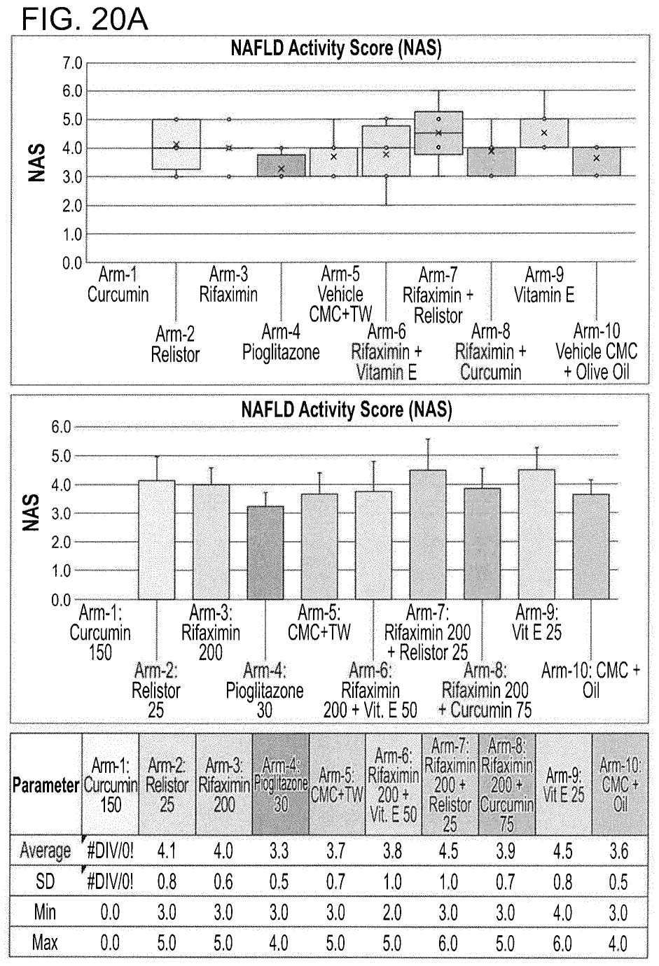

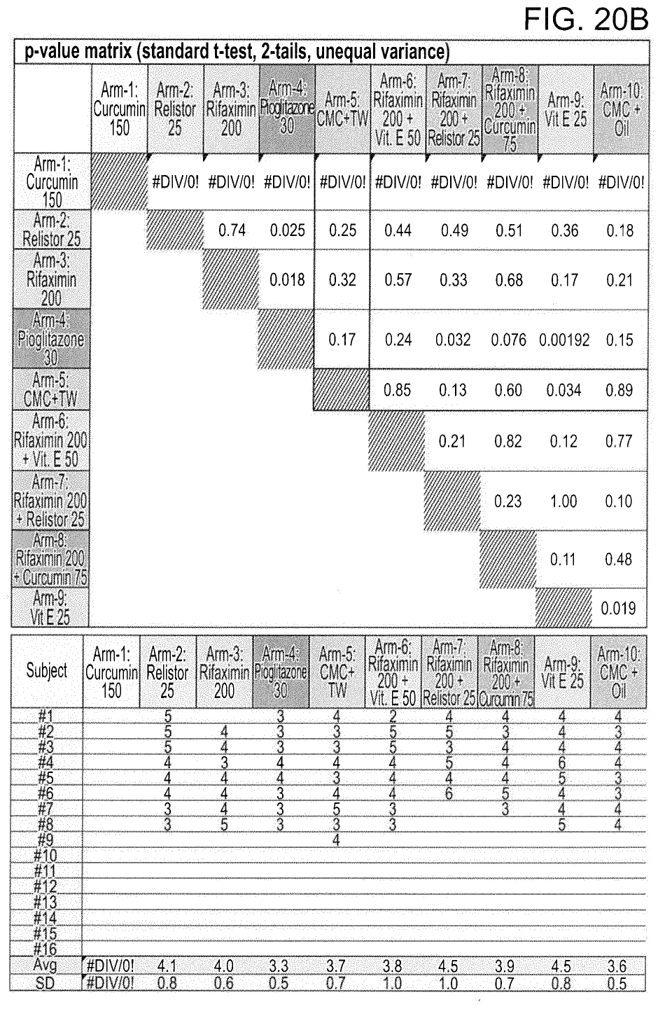

FIGS. 20A-20B shows NAFLD Activity Score (NAS).

### Steatosis, Activity and Fibrosis Score (SAF)

The only statistically significant difference in SAF was for a slight increase in Arm-9 (Vitamin E, 50 mg, p=0.034) when compared to the control Arm (Arm-5; WDSW+CMC-Tween). Pioglitazone, again, was the only treatment trending toward reducing SAF (all other treatment groups tended to non-significantly increase SAF) although not in a statistically significant way.

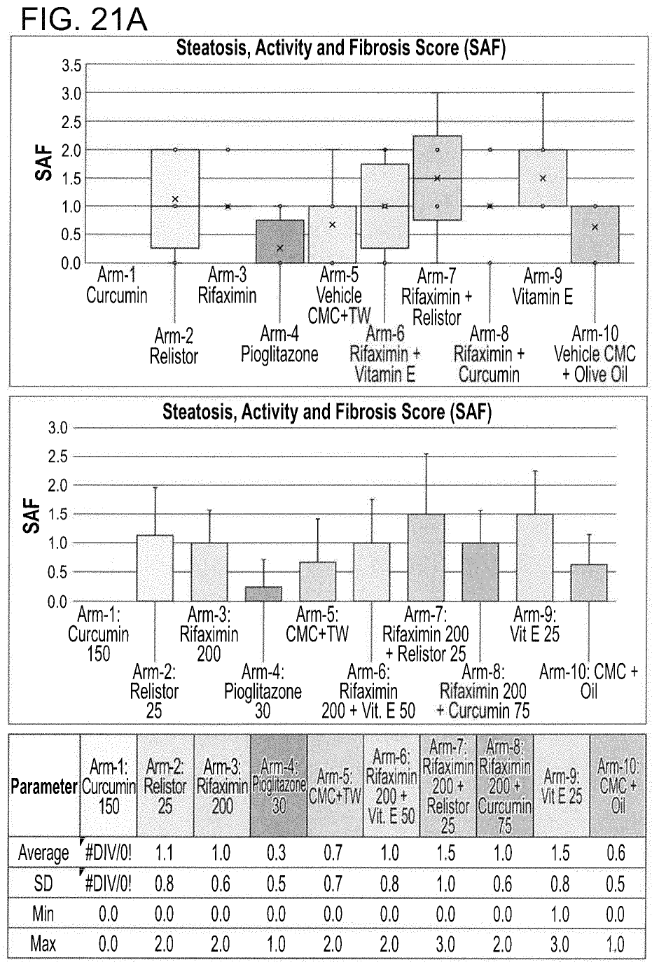

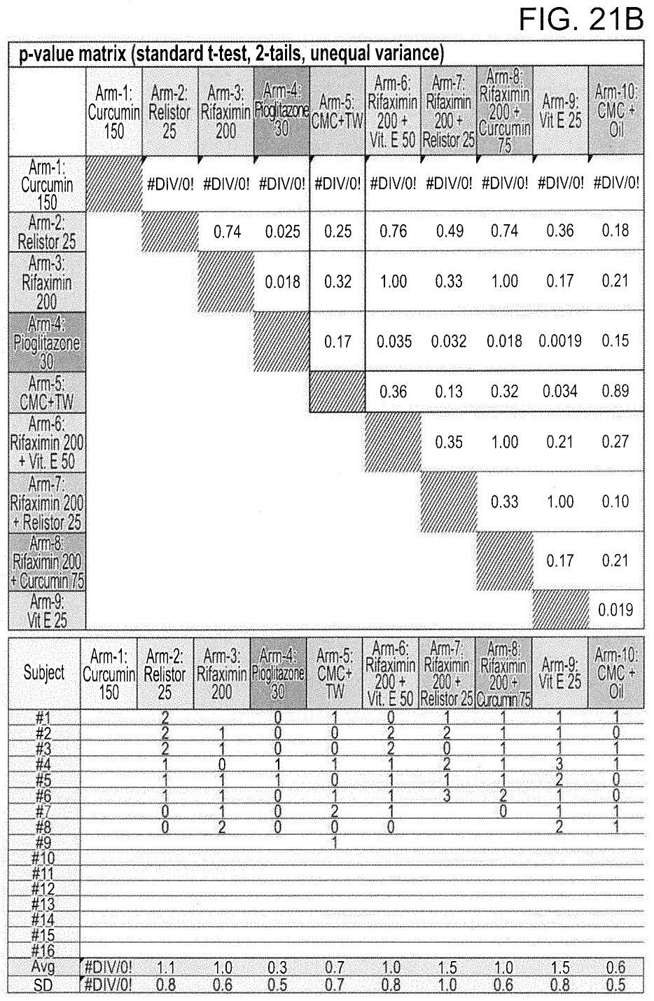

FIGS. 21A-21B shows SAF Score.

### Liver Fibrosis

There were no statistical differences in Liver Fibrosis between any groups; likely due to low level of liver fibrosis and relatively high level of variability.

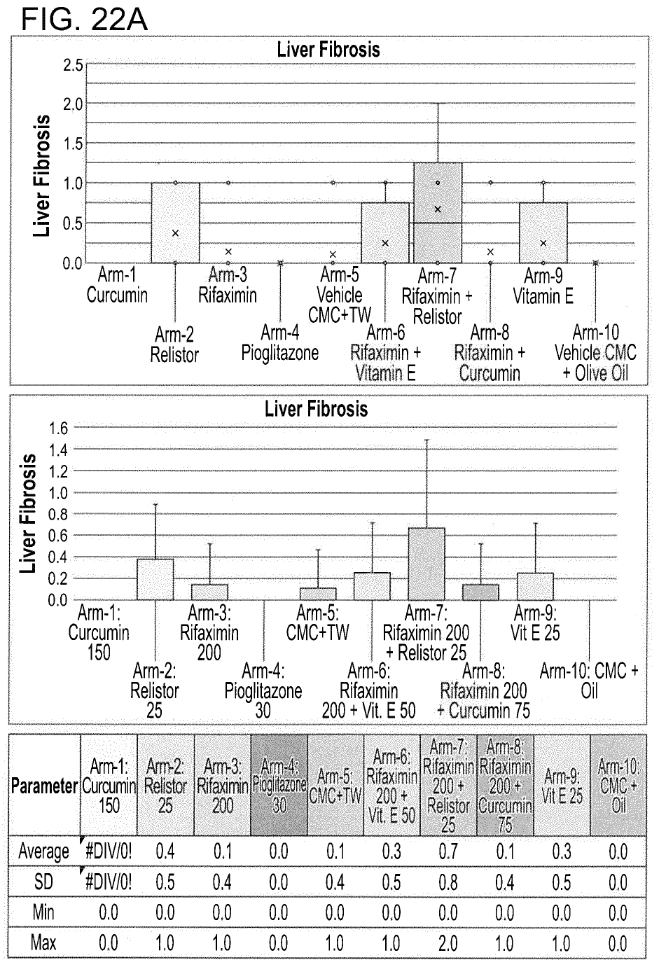

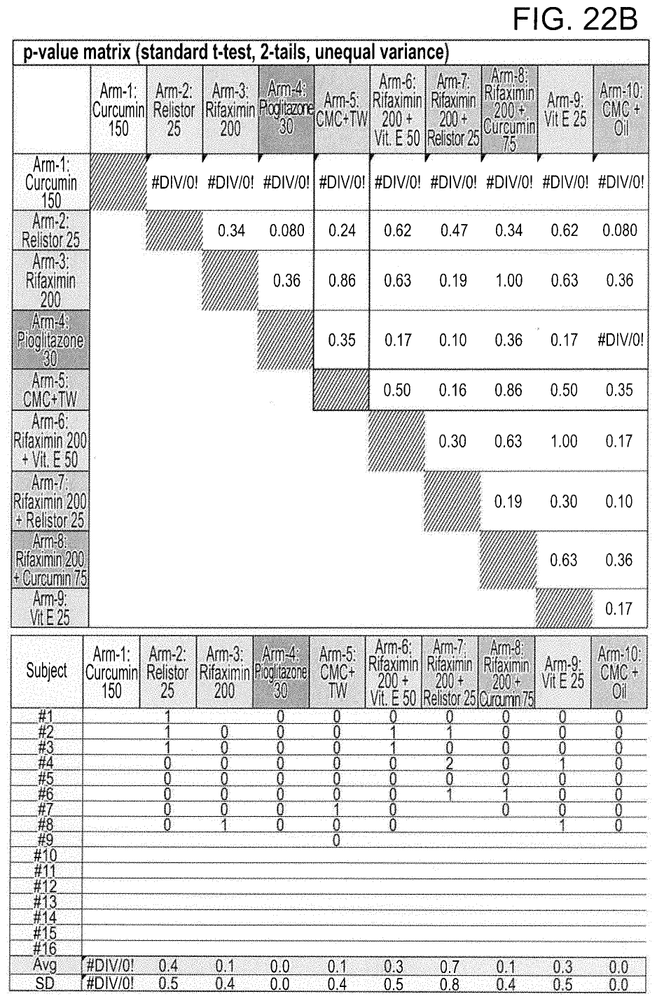

FIGS. 22A-22B shows liver fibrosis.

### Perisinusoidal Fibrosis

The only statistically significant difference in Perisinusoidal Fibrosis was for a significant/complete decrease in Perisinusoidal Fibrosis in the Pioglitazone Arm (Arm-4, p=0.035) when compared to the control Arm (Arm-5; WDSW+CMC-Tween).

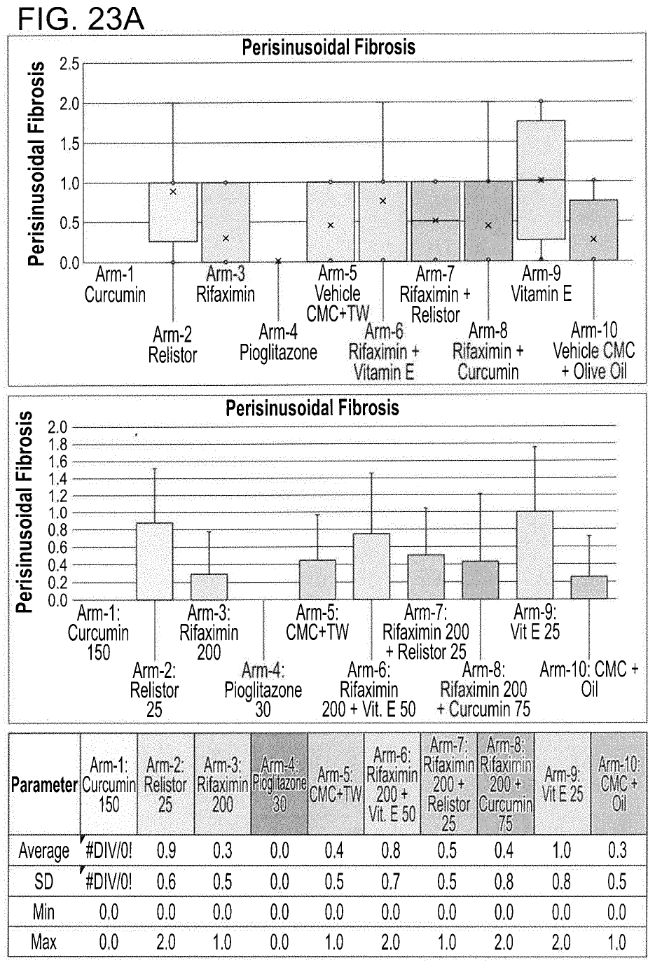

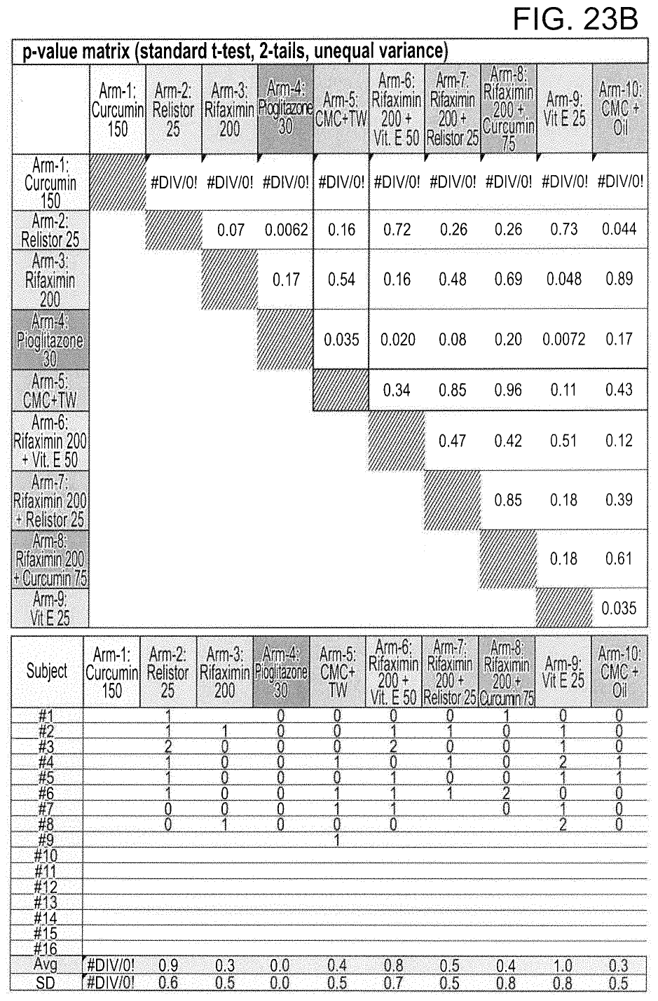

FIGS. 23A-23B shows perisinusoidal fibrosis.

### Disease Category

No group had “Normal” histology; a likely and expected outcome of having all groups fed the WDSW diet.

Steatosis was the main diagnostic feature in two control groups (Arm-5: CMC+Tween and Arm-10: CMC+Olive oil) with 89% and 100% steatosis, respectively.

Only the Pioglitazone Arm (Arm-4) had steatosis-only (100%) at the end of treatment; all other groups had a mix of steatosis (predominantly) and NASH (from 11% to 50%).

MNTX and then Vitamin E were the groups with the highest prevalence of NASH (38% in Arm-2: MNTX alone and 50% in Arm-7: MNTX+Rifaximin); however, Rifaximin probably did not contribute since in Arm-3 (Rifaximin), the proportion of Steatosis and NASH was similar to the control group (Arm-5). However, it is noted that an increase in gut permeability is linked to the development or progression of NASH (in humans) by allowing gut microbiome end products to reach and harm the liver. In the present study, this microbiome-liver axis does not appear to be at play. It is possible that the microbiome in the DIAMOND™ mice in this study with this diet was not eliciting the generation of harmful compounds that would trigger the development of NASH, as would be expected to occur in a human subject. A decrease in the prevalence of NASH is expected in a mouse model where a human-like microbiome-liver axis is present.

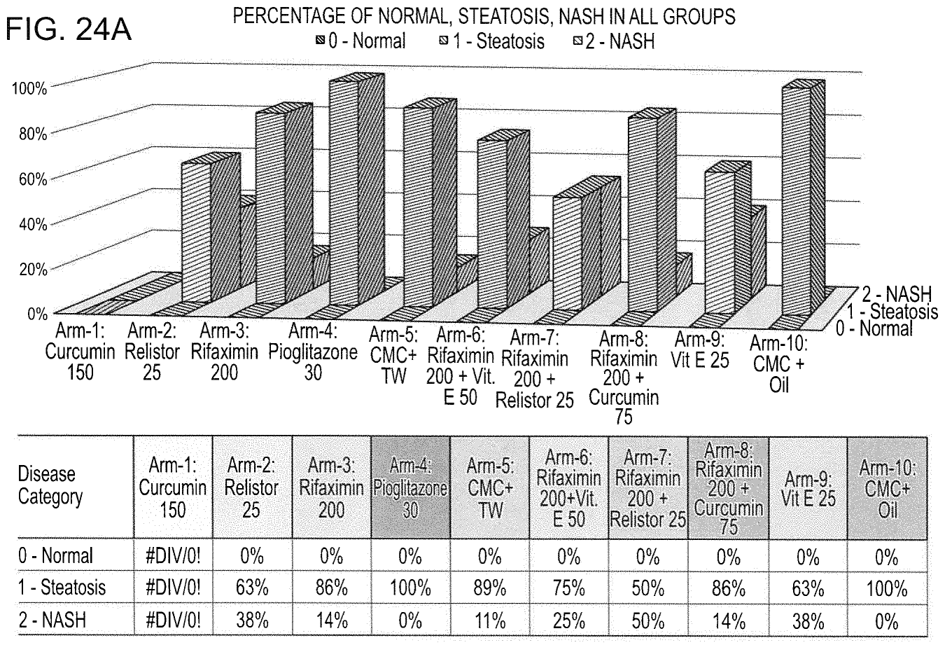

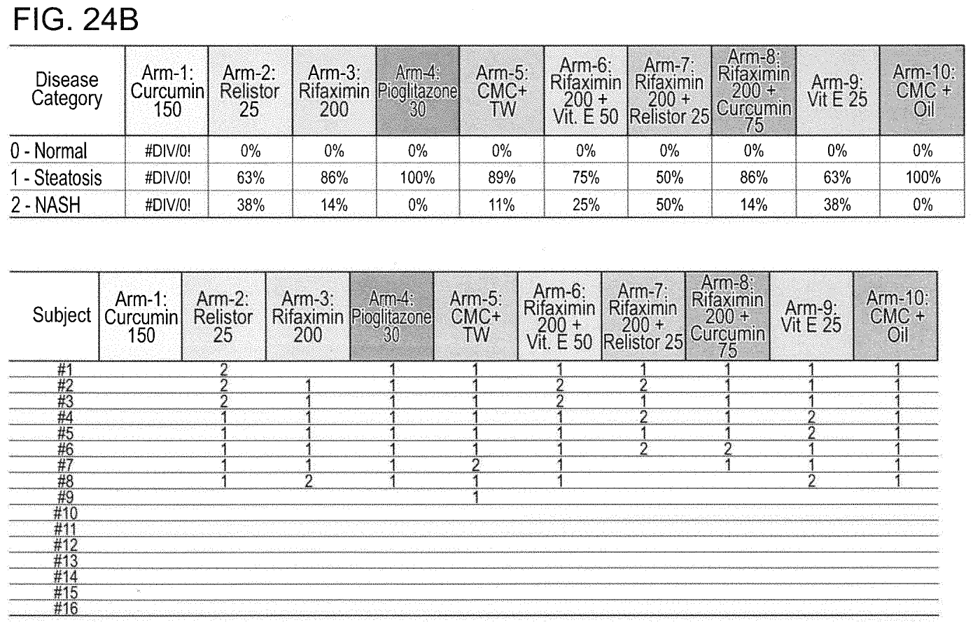

FIG. 24A shows the percentage of Disease Categories in each group at end of study. (0=Normal histology, 1=Steatosis, 2=NASH).

FIG. 24B shows the percentage of Disease Categories in each group at end of study and individual disease category classification for each mouse in each group (0=Normal histology, 1=Steatosis, 2=NASH).

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating increased gut permeability, a disease or condition associated with increased gut permeability, and/or reducing gut permeability in a subject in need thereof, the method comprising administering a therapeutic agent selected from the group consisting of methylnaltrexone, or a salt thereof, rifaximin, and a combination thereof.

2.-3. (canceled)

4. The method of claim 1, wherein the disease or condition associated with increased gut permeability is selected from the group consisting of irritable bowel syndrome, inflammatory bowel disease, Crohn's disease, ulcerative colitis, indeterminate colitis, chemotherapy-induced colitis, celiac disease, infectious diarrhea, atopy, allergy, food allergy, asthma, autism, chronic fatigue syndrome, lupus, metabolic syndromes, diabetes, type 1 diabetes, type 2 diabetes, hypertension, hyperlipidemia, neoplasia, cancer, idiopathic inflammatory conditions, rheumatoid arthritis, neurologic disorders, multiple sclerosis, migraines, psoriatic arthritic, autoimmune diseases, rheumatoid arthritis, skin disorders, psoriasis, eczema, acne, metabolic bone disease, osteoporosis in adults, primary growth failure in children, liver disease, fatty liver diseases, nonalcoholic steatohepatitis (NASH), nonalcoholic fatty liver disease (NAFLD), hepatic encephalopathy (HE), ankylosing spondylitis, obesity, graft versus host disease (GVHD), multiple organ dysfunction syndrome, Parkinson's disease, acute pancreatitis, fibromyalgia, mental illness, depression, schizophrenia, burn injury, heart failure, and renal failure.

5. The method of claim 4, wherein the disease or condition associated with increased gut permeability is a liver disease, optionally, a fatty liver disease, optionally, selected from the group consisting of NASH and NAFLD.

6.-7. (canceled)

8. The method of claim 4, wherein the disease or condition associated with increased gut permeability is hepatic encephalopathy (HE).

9. A method of treating NASH, NAFLD, and/or a symptom thereof in a subject in need thereof, the method comprising administering a therapeutic agent selected from the group consisting of methylnaltrexone, or a salt thereof, rifaximin, and a combination thereof.

10. (canceled)

11. The method of claim 9, wherein the symptom is increased gut permeability.

12. The method of claim 1, wherein the subject is administered methylnaltrexone or a salt thereof.

13. The method of claim 1, wherein the subject is administered (R)-N-methylnaltrexone bromide, oral Relistor or subcutaneous Relistor.

14.-15. (canceled)

16. The method of claim 1, wherein the subject is administered rifaximin.

17. The method of claim 1, wherein the subject is administered rifaximin orally.

18. The method of claim 1, wherein the subject is administered (R)-N-methylnaltrexone bromide and rifaximin.

19. The method of claim 1, comprising orally administering about 50 mg to about 600 mg, about 100 mg to about 500 mg, or about 150 mg to about 450 mg of methylnaltrexone, or a salt thereof and/or about 1 mg/kg to about 100 mg/kg, about 5 mg/kg to about 75 mg/kg, about 15 mg/kg to about 60 mg/kg, or about 25 mg/kg to about 50 mg/kg of methylnaltrexone, or a salt thereof.

20. (canceled)

21. The method of claim 1, comprising orally administering (i) about 25 mg to about 1000 mg, about 300 mg to about 750 mg, or about 500 mg to about 600 mg; or (ii) about 100 mg/kg to about 700 mg/kg, about 250 mg/kg to about 550 mg/kg or about 350 mg/kg to about 450 mg/kg of rifaximin, or a salt thereof.

22. A pharmaceutical composition for treating increased gut permeability, treating NASH or a symptom thereof, treating NAFLD or a symptom thereof, treating a disease or condition associated with increased gut permeability, and/or reducing gut permeability in a subject in need thereof, the pharmaceutical composition comprising a therapeutic agent selected from the group consisting of methylnaltrexone, or a salt thereof, rifaximin, and a combination thereof.

23.-26. (canceled)

27. The pharmaceutical composition of claim 22, wherein the symptom is increased gut permeability.

28. The pharmaceutical composition of claim 22, wherein the pharmaceutical composition comprises methylnaltrexone, or a salt thereof.

29. The pharmaceutical composition of claim 22, wherein the pharmaceutical composition comprises (R)-N-methylnaltrexone bromide, oral Relistor or subcutaneous Relistor.

30.-31. (canceled)

32. The pharmaceutical composition of claim 22, wherein the pharmaceutical composition comprises rifaximin.

33. The pharmaceutical composition of claim 22, wherein the pharmaceutical composition comprises an oral dosage of rifaximin.

34. The pharmaceutical composition of claim 22, wherein the pharmaceutical composition comprises (R)-N-methylnaltrexone bromide and rifaximin.

35. The pharmaceutical composition of claim 22, wherein the pharmaceutical composition comprises about 1 mg/kg to about 100 mg/kg, about 5 mg/kg to about 75 mg/kg, about 15 mg/kg to about 60 mg/kg, or about 25 mg/kg to about 50 mg/kg of methylnaltrexone, or a salt thereof; and/or about 1 mg/kg to about 50 mg/kg, about 5 mg/kg to about 30 mg/kg, or about 15 mg/kg to about 25 mg/kg of methylnaltrexone, or a salt thereof.

36. (canceled)

37. The pharmaceutical composition of claim 22, wherein the pharmaceutical composition comprises (i) about 25 mg to about 1000 mg, about 300 mg to about 750 mg, or about 500 mg to about 600 mg; or (ii) about 100 mg/kg to about 700 mg/kg, about 250 mg/kg to about 550 mg/kg or about 350 mg/kg to about 450 mg/kg of rifaximin, or a salt thereof.
